(19) United States
(12) Patent Application Publication
(10) Pub. No.: US 2026/0337564 A1
(43) Pub. Date: Oct. 29, 2026

(54) ALPHA BETA T CELL COMPOSITIONS AND METHODS OF USE

(71) Applicant: BeOne Medicines I GmbH, Basel (CH)

(72) Inventors: Jhang-Sian YU, Taipei City (TW); Shih-Min HUANG, Taipei City (TW); Lien-Szu WU, Taipei City (TW); Jin-Yi LU, Taipei City (TW); Ao HU, Shanghai (CN); Yi-Han DAI, Taipei City (TW); Chia-Hung LIN, Taipei City (TW); Minhong YAN, Shanghai (CN); Yo-Chuen LIN, Taipei City (TW)

(73) Assignee: BeOne Medicines I GmbH, Basel (CH)

(21) Appl. No.: 19/650,163

(22) Filed: Apr. 16, 2026

(30) Foreign Application Priority Data
Apr. 17, 2025 (WO) PCT/CN2025/089599
Mar. 9, 2026 (WO) PCT/CN2026/082246

(51) Int. Cl. C12N 5/0783; A61K 40/11; A61K 40/31; A61K 40/42; C07K 14/705; C07K 16/28; C12N 9/22; C12N 15/11

(52) U.S. Cl. CPC C12N 5/0636; A61K 40/11; A61K 40/31; A61K 40/4211; C07K 14/7051; C07K 16/2803; C12N 9/226; C12N 15/11; C12N 2310/20; C12N 2310/321; C12N 2510/00

(57) ABSTRACT

The present disclosure provides genetically modified iPSC-derived αβT cells and their precursors. A double genomic disruption in the suppressor of cytokine signaling 1 (SOCS1) gene and the cytokine-inducible sh2-containing protein (CISH) gene are provided, as is a triple genomic disruption in genes for SOCS1, CISH, and Bcl-2 interacting mediator of cell death (BIM), as is a quadruple genomic disruption in genes for SOCS1, CISH, BIM, and cell surface death receptor (FAS), as is a quintuple genomic disruption in genes for SOCS1, CISH, BIM, β-2-Microglobulin (B2M), and class II transactivator (CIITA), as is a sextuple genomic disruption in genes for SOCS1, CISH, BIM, B2M, CIITA, and FAS. Also provided is genetically modified iPSC-derived αβT cells and their precursors with improved proliferation and tumor killing activity. Also provided are genetically modified iPSC-derived αβT cells and their precursors further comprising anti-CD19 CAR. The present disclosure further provides methods making and using such cells, as well as gene editing systems.

### CROSS-REFERENCE TO RELATED APPLICATIONS

This application claims priority to and the benefit of International Patent Application No. PCT/CN2025/089599, filed Apr. 17, 2025, and International Patent Application No. PCT/CN2026/082246, filed Mar. 9, 2026, the contents of which are hereby incorporated herein by reference in their entirety.

### SEQUENCE LISTING

The present application contains a sequence listing which has been submitted electronically in xml format, and is hereby incorporated by reference in its entirety. Said xml file was created on Apr. 16, 2026, is 90,275 bytes in size, and named 138881_1623_Sequence_Listing.xml.

### FIELD OF THE DISCLOSURE AND BACKGROUND

This disclosure relates to genetically modified αβT cells and their precursors. An alpha-beta T cell (αβT-cell) is a type of T cell that has a T-cell receptor (TCR) composed of a α glycoprotein chain and a β glycoprotein chain on the surface of the cell and is activated in a major histocompatibility complex-dependent manner. αβT-cells can be genetically modified to tailor the αβT-cell for specific roles in diagnostic and therapeutic settings.

Human BIM gene (Gene ID: 10018), also called BCL2L11, or “Bcl-2 Interacting Mediator of cell death” is transcribed as three splice variants: BIM_{EL}, BIM_{L}, and BIM_{S}. BIM is a member of BH3 (Bcl-2 homology 3)-only proteins, which can directly activate the proapoptotic effector proteins BAX and BAK. On the other hand, BIM also can indirectly activate BAX and BAK by binding to antiapoptotic BCL-2 family members (BCL-2, BCL-XL, MCL-1 and A1).

Cytokine-inducible SH2-containing protein (CISH)/(CIS1) (Gene ID: 1154, NM_013324.7, NP_037456.5) is a member of the suppressor of cytokine signaling (SOCS) protein family. The SOCS family members are negative regulators of cytokine signal transduction that inhibit the JAK/STAT pathway. Each SOCS family member contains a central SH2 domain and a conserved carboxy-terminal motif designated as the SOCS box. These proteins are important regulators of cytokine signaling, proliferation, differentiation, and immune responses.

The SOCS family consists of at least eight members including the originally identified CIS1, as well as SOCS1, SOCS2, SOCS3, SOCS4, SOCS5, SOCS6, and SOCS7. SOCS1 (Suppressor of cytokine signaling 1) also known as JAB (Janus kinase binding protein), SSI-1 (Stat-induced Stat inhibitor-1), Gene ID: 8651, NM_003745.2, NP_003736.1, and TIP3 (Tec-interacting protein 3), is a cytokine-regulated SOCS family member that directly inhibits JAK family members through interaction within their kinase activation loop. In addition to inhibiting JAK/STAT signaling, SOCS1 can also negatively regulate Toll-like receptors that contribute to innate immunity.

Human FAS cell surface death receptor protein (Gene ID: 355, NM_000043.6, NP_000034.1), also known as “FAS” or “CD95,” is one of five tumor necrosis factor (TNF) superfamily death receptors. Human FAS can induce caspase-dependent apoptosis following engagement with its extracellular ligand (FAS ligand (FASL)). The FASL-FAS signaling-induced apoptotic pathway plays an important role in immune regulation, development of cancer, and progression of cancer. In addition, FASL-FAS signaling also has a role in the auto-regulatory circuit for CAR-engineered lymphocyte persistence.

β-2-Microglobulin (Gene ID: 567, NM_004048, NP_004039), also known as “β2M” or “β2M”, is a component of the human leukocyte antigen (HLA) class I molecules found on the surface of nearly all nucleated cells. The β2M gene, which encodes β2M, is located on chromosome 15 (15q21.1) and consists of 4 exons. It encodes a 12 kDa protein. β2M associates non-covalently with the HLA class I heavy chain, enabling proper folding and stabilization of HLA class I. B2M-free HLA class I molecules are eventually degraded by proteasomes in the cytosol. Genetic ablation of the β2M gene eliminates surface HLA class I expression, reducing CD8+ T cell-mediated allogeneic responses.

The Class II transactivator gene (Gene ID: 4261, NM_001286402.1, NP_001273331.1), encodes the Class II transactivator protein, which is also called “CIITA”. The CIITA gene is located on chromosome 16 (16p13.13) and has 19 exons. It encodes a 130 kDa protein. CIITA serves as a master regulator of HLA class II expression by recruiting and facilitating the assembly of DNA-binding proteins including the regulatory factor X (RFX) complex, cAMP response element-binding protein (CREB), and nuclear factor Y (NF-Y). Together, this complex enables HLA class II expression. Genetic ablation of the CIITA gene eliminates surface HLA class II expression, reducing CD4+ T cell-mediated allogeneic responses.

Allogeneic immune recognition and rejection remain obstacles in cellular transplantation and cell therapy development. A major cause of transplant rejection is HLA mismatch. To suppress these HLA mismatch-mediated responses, genetic ablation of HLA in iPSC-derived αβ T cells can be conducted.

Interferon-gamma receptor (IFNGR) is a cytokine receptor of interferon gamma (IFN-γ). IFNGR comprises a heterodimer of interferon-gamma receptor 1 (IFNGR1) and interferon-gamma receptor 2 (IFNGR2). IFN-γ can signal IFNGR-expressing cells, enabling the immunomodulatory effects of IFN-γ, including augmenting antigen presentation through both the MHC class I and class II pathways. CD132, also called “the common γ chain (γc) of interleukin-2 receptor” or “IL2RG,” is a cytokine receptor subunit that is common to the cytokine receptor complexes of at least IL-2, IL-4, IL-7, IL-9, IL-15, and IL-21. Interleukin-2 receptor beta subunit, also known as “CD122” or “IL2RB,” is a cytokine receptor subunit of the cytokine receptor complex of IL-2 (IL2R). IL2R is a protein complex found on the surface of cells, including T cells, NK cells and other immune cells. IL-2 mediated IL2R signaling promotes the differentiation of T cells into effector T cells and memory T cells. IL-2 mediated IL2R signaling enables downstream signaling pathways through the Janus kinase (JAK)/signal transducer and activator of transcription (STAT) pathway, e.g., STAT1, STAT5. Modulating these signaling pathways may, for example, be useful in treating diseases such as cancer by helping to promote killing of tumor cells by immune cells.

There remains an unmet need for therapeutic αβT-cells. Such modified cells would be useful in a variety of diagnostic and therapeutic settings, such as in the treatment of human disease and disorders.

### SUMMARY

The present inventors have herein demonstrated that genomic disruptions in 1) an endogenous suppressor of cytokine signaling 1 (SOCS1) gene; and 2) an endogenous cytokine-inducible SH2-containing protein (CISH) gene, and optionally 3) an endogenous Bcl-2 Interacting Mediator of cell death (BIM) gene, and optionally 4) an endogenous FAS cell surface death receptor (FAS) gene, and optionally, 5) an endogenous β-2-Microglobulin (B2M) gene, and optionally 6) an endogenous class II transactivator (CIITA) gene in αβT (e.g., iPSC-derived αβT) cells confer enhanced cytotoxicity (including enhanced secretion of effector enzymes, e.g., Granzyme A and Granzyme B) and persistence, synergistic effect in enhancing cytokine release (e.g., IFN-g) and/or increased survival in the αβT cells. The modified cells can be used for a variety of therapeutic and diagnostic purposes.

In some embodiments, the present disclosure provides a modified αβT cell (e.g., from primary αβT cell or differentiated from modified iPSC), comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein, which shows synergistic effects in enhancing cytokine (e.g., IFN-g) release and better effects in promoting αβT persistence and/or enhanced αβT cytotoxicity (including enhanced secretion of effector enzymes, e.g., Granzyme A and Granzyme B), compared with an unmodified control.

In some embodiments, the present disclosure provides a modified αβT (e.g., from primary αβT cell or differentiated from modified iPSC), comprising a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell, showing better survival ability of αβT, compared with an unmodified control.

In some embodiments, the present disclosure provides a modified αβT (e.g., from primary αβT cell or differentiated from modified iPSC), comprising (1) a genomic disruption in an endogenous suppressor of cytokine signaling 1 (SOCS1) gene that suppresses or eliminates expression of a functional SOCS1 protein, (2) a genomic disruption in an endogenous cytokine-inducible SH2-containing protein (CISH) gene that suppresses or eliminates expression of a functional CISH protein, and (3) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell, showing synergistic effect in enhancing cytokine (e.g., IFN-g) releasing, enhanced αβT persistence, enhanced αβT cytotoxicity and better survival ability of αβT cells, compared with an unmodified control.

In some embodiments, the present disclosure provides a modified αβT cell (e.g., from primary αβT cell or differentiated from modified iPSC), comprising (1) a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein, and/or (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell, optionally further comprising (a) a genomic disruption in an endogenous FAS cell surface death receptor (FAS) gene, optionally further comprising (b) a genomic disruption in an endogenous β-2-Microglobulin (B2M) gene, optionally further comprising (c) a genomic disruption in an endogenous Class II transactivator (CIITA) gene, optionally further comprising (d) one or more exogenous polynucleotides encoding a signal converter, and optional further comprising (e) an exogenous polynucleotide ending a suicide switch, showing any one or more of the following features:
(1) enhanced αβT persistence and/or enhanced αβT cytotoxicity (including enhanced secretion of effector enzymes, e.g., Granzyme A and Granzyme B);
(2) enhanced survival ability of αβT;
(3) enhanced durable killing (e.g., around 18 rounds of killing in one embodiment);
(4) synergistic effect in enhancing cytokine (e.g., IFN-g) release;
(5) improved cell expansion;
(6) reduced CRS-related cytokine release; and
(7) allowing inducible cell death as a safety mechanism.

The present disclosure also provides a modified iPSC or an intermediate cell differentiated from iPSC having 1) a genomic disruption in an endogenous suppressor of cytokine signaling 1 (SOCS1) gene that suppresses or eliminates expression of SOCS1 and a genomic disruption in an endogenous cytokine-inducible SH2-containing protein (CISH) gene that suppresses or eliminates expression of CISH, and/or (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell, optionally further comprising (a) a genomic disruption in an endogenous FAS cell surface death receptor (FAS) gene, optionally further comprising (b) a genomic disruption in an endogenous β-2-Microglobulin (B2M) gene, optionally further comprising (c) a genomic disruption in an endogenous Class II transactivator (CIITA) gene, and/or optionally further comprising (d) one or more exogenous polynucleotides encoding a signal converter, which can be used for differentiation into the modified αβT cells.

In some aspects, the genomic modifications herein (e.g., a genomic disruption in SOCS1, a genomic disruption in CISH, a genomic disruption in BIM, a genomic disruption in FAS, and/or introduction of a signal converter) contribute to effector enhancement of αβ T cell, thereby providing a functionally augmented αβ T cell platform. In some aspects, the modified αβ T cells are further combined with an exogenous recognition module, such as a chimeric antigen receptor, to enable antigen-specific target cell recognition and killing.

The above-mentioned modified iPSC, αβT cell, or intermediate cell differentiated from an iPSC can serve as a platform cell for introduction of an antigen receptor, such as chimeric antigen receptor (CAR) or T cell receptor (TCR).

The above-mentioned modified iPSC, αβT cell, or intermediate cell differentiated from an iPSC can further comprise one or more exogenous polynucleotides encoding a TCR, e.g., a TCR specifically binding to KRAS G12V.

The above-mentioned modified iPSC, αβT cell, or intermediate cell differentiated from an iPSC can further comprise an exogenous polynucleotide encoding a chimeric antigen receptor (CAR).

The above-mentioned modified iPSC, αβT cell, or intermediate cell differentiated from an iPSC can further comprise an exogenous polynucleotide encoding a chimeric antigen receptor (CAR) which specifically binds human CD19 and/or an exogenous polynucleotide encoding a chimeric antigen receptor (CAR) which specifically binds human CD20.

The CAR incorporated in the modified αβT cell described herein functions to recognize tumor-associated antigens (TAAs) expressed on the surface of cancer cells.

In some embodiments, the present disclosure provides a modified αβT cell (e.g., from primary αβT cell or differentiated from modified iPSC), comprising (a) an exogenous polynucleotide encoding a chimeric antigen receptor (CAR) (e.g., anti-CD19 CAR and/or an anti-CD20 CAR) or a TCR; in combination with (b) any one or more of:

(1) a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein; (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell; (3) a genomic disruption in an endogenous FAS cell surface death receptor (FAS) gene; (4) a genomic disruption in an endogenous β-2-Microglobulin (B2M) gene; (5) a genomic disruption in an endogenous Class II transactivator (CIITA) gene; and (6) one or more exogenous polynucleotides encoding a signal converter,
showing any one or more of the following features:
(1) enhanced αβT persistence and/or enhanced αβT cytotoxicity (including enhanced secretion of effector enzymes, e.g., Granzyme A and Granzyme B);
(2) enhanced survival ability of αβT;
(3) enhanced durable killing (e.g., around 18 rounds of killing in one embodiment);
(4) synergistic effect in enhancing cytokine (e.g., IFN-g) release; and
(5) improved cell expansion; and
(6) reduced CRS-related cytokine release.

The present disclosure encompasses, inter alia, the following embodiments, among others disclosed herein.

1. A modified iPSC, αβT cell, or intermediate cell differentiated from an iPSC, comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein.
2. The modified cell of embodiment 1, wherein the genomic disruption in the endogenous SOCS1 gene is in exon 2 of the SOCS1 gene; optionally wherein the genomic disruption is an inactivating nucleic acid mutation, optionally wherein the mutation is an insertion, deletion, or substitution.
3. The modified cell of embodiment 1 or embodiment 2, wherein the genomic disruption in the endogenous CISH gene is in exon 3 or exon 4 of the CISH gene; optionally wherein the genomic disruption is an inactivating nucleic acid mutation, optionally wherein the mutation is an insertion, deletion, or substitution.
4. The modified cell of any one of embodiments 1-3, wherein the genomic disruption in the endogenous SOCS1 gene is within a target sequence in SOCS1 that comprises SEQ ID NO: 17 or SEQ ID NO: 23 and/or the genomic disruption in the endogenous CISH gene is within a target sequence in CISH that comprises SEQ ID NO: 15, SEQ ID NO: 16 or SEQ ID NO: 24.
5. The modified cell of any one of embodiments 1-4, wherein the genomic disruption within SOCS1 and/or CISH is made by administering guide RNAs to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
6. The modified cell of embodiment 5, wherein the guide RNAs comprise the guide sequence of any one or more of SEQ ID NOs: 5, 11, 3, 4, and 12, or comprises the sgRNA or crRNA sequence of any one or more of SEQ ID NOs: 29, 37, 27, 28, and 38.
7. The modified cell of embodiment 5 or embodiment 6, wherein (1) the endonuclease is the MAD7 endonuclease, and the guide RNAs comprise the guide sequence of SEQ ID NO: 11, or comprise the crRNA sequence of SEQ ID NO: 37; or (2) the endonuclease is the Cas9 endonuclease, and the guide RNAs comprise the guide sequence of SEQ ID NO: 5, or comprise the sgRNA sequence of SEQ ID NO: 29.
8. The modified cell of embodiment 5 or embodiment 6, wherein (1) the endonuclease is the MAD7 endonuclease, and the guide RNAs comprise the guide sequence of SEQ ID NO: 12, or comprise the crRNA sequence of SEQ ID NO: 38; or (2) the endonuclease is the Cas9 endonuclease, and the guide RNAs comprise the guide sequence of any one of SEQ ID NOs: 3 and 4, or comprise the sgRNA sequence of any one of SEQ ID NOs: 27 and 28.
9. The modified cell of any one of embodiments 1-8, further comprising a genomic disruption in an endogenous Bcl-2 Interacting Mediator of cell death (BIM) gene.
10. The modified cell of embodiment 9, wherein the genomic disruption in the endogenous BIM gene is in exon 2C of the BIM gene.
11. The modified cell of embodiment 9 or embodiment 10, wherein the expression and/or function of BIM_{EL} and BIM_{L} splice variants are reduced in the modified cell relative to that in an unmodified control and the expression and/or function of BIM_{S} splice variant is retained in the modified cell.
12. The modified cell of any one of embodiments 9-11, wherein the genomic disruption in the endogenous BIM gene is an inactivating nucleic acid mutation in an exon 2C of an endogenous BIM gene, optionally wherein the mutation is an insertion, deletion, or substitution.
13. The modified cell of any one of embodiments 9-12, wherein the genomic disruption in the endogenous BIM gene is within a target sequence in BIM that comprises any one of SEQ ID NOs: 14, 18, 19, 20, 21, and 22.
14. The modified cell of any one of embodiments 9-13, wherein the genomic disruption within BIM is made by administering a guide RNA to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
15. The modified cell of embodiment 14, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
16. The modified cell of embodiment 14 or embodiment 15, wherein (1) the endonuclease is the MAD7 endonuclease, and the guide RNAs comprise the guide sequence of SEQ ID NO: 10, or comprise the crRNA sequence of SEQ ID NO: 36; or (2) the endonuclease is the Cas9 endonuclease, and the guide RNAs comprise the guide sequence of any one of SEQ ID NOs: 2, 6, 7, 8, and 9, or comprises the sgRNA sequence of any one of SEQ ID NOs: 26, 32, 33, 34, and 35.
17. The modified cell of any one of embodiments 1-16, further comprising a genomic disruption in an endogenous FAS cell surface death receptor (FAS) gene that suppresses or eliminates expression of a functional FAS protein.
18. The modified cell of embodiment 17, wherein the genomic disruption in the FAS gene is in exon 1 of the FAS gene.
19. The modified cell of embodiment 17 or embodiment 18, wherein the genomic disruption is an inactivating nucleic acid mutation in exon 1 of an endogenous FAS gene, optionally wherein the mutation is an insertion, deletion, or substitution.
20. The modified cell of any one of embodiments 17-19, wherein the genomic disruption is within a target sequence in FAS that comprises SEQ ID NO: 62.
21. The modified cell of any one of embodiments 17-20, wherein the genomic disruption within FAS is made by administering a guide RNA to said cell, and optionally wherein an endonuclease or a nucleic acid encoding the endonuclease is also administered to said cell.
22. The modified cell of embodiment 21, wherein the guide RNA comprises the guide sequence of SEQ ID NO: 61, or comprises the sgRNA sequence of SEQ ID NO: 60.
23. The modified cell of any one of embodiments 1-22, further comprising a genomic disruption in an endogenous β-2-Microglobulin (B2M) gene that suppresses or eliminates expression of a functional β2M protein.
24. The modified cell of embodiment 23, wherein the genomic disruption in the β2M gene is in exon 2 of the β2M gene.
25. The modified cell of embodiment 23 or embodiment 24, wherein the genomic disruption is an inactivating nucleic acid mutation in exon 2 of an endogenous β2M gene, optionally wherein the mutation is an insertion, deletion, or substitution.
26. The modified cell of any one of embodiments 23-25, wherein the genomic disruption is within a target sequence in β2M that comprises SEQ ID NO: 56.
27. The modified cell of any one of embodiments 23-26, wherein the genomic disruption within β2M is made by administering a guide RNA to said cell, and optionally wherein an endonuclease or a nucleic acid encoding the endonuclease is also administered to said cell.
28. The modified cell of embodiment 27, wherein the guide RNA comprises the guide sequence of SEQ ID NO: 55, or comprises the sgRNA sequence of SEQ ID NO: 54.
29. The modified cell of any one of embodiments 1-28, further comprising a genomic disruption in an endogenous Class II transactivator (CIITA) gene that suppresses or eliminates expression of a functional CIITA protein.
30. The modified cell of embodiment 29, wherein the genomic disruption in the CIITA gene is in exon 3 of the CIITA gene.
31. The modified cell of embodiment 29 or embodiment 30, wherein the genomic disruption is an inactivating nucleic acid mutation in exon 3 of an endogenous CIITA gene, optionally wherein the mutation is an insertion, deletion, or substitution.
32. The modified cell of any one of embodiments 29-31, wherein the genomic disruption is within a target sequence in CIITA that comprises SEQ ID NO: 59.
33. The modified cell of any one of embodiments 29-32, wherein the genomic disruption within CIITA is made by administering a guide RNA to said cell, and optionally wherein an endonuclease or a nucleic acid encoding the endonuclease is also administered to said cell.
34. The modified cell of embodiment 33, wherein the guide RNA comprises the guide sequence of SEQ ID NO: 58, or comprises the sgRNA sequence of SEQ ID NO: 57.
35. The modified cell of any one of embodiments 1-34, further comprising a genomic disruption in an endogenous T-cell receptor alpha constant region (TRAC) gene that suppresses or eliminates expression of a functional TRAC protein and/or a genomic disruption in an endogenous TRBC1/2 gene that suppresses or eliminates expression of a functional TRBC1/2 protein.
36. The modified cell of any one of embodiments 1-35, wherein the genomic disruption is an inactivating nucleic acid mutation, optionally wherein the mutation is an insertion, deletion, or substitution.
37. The modified cell of any one of embodiments 1-36, further comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).
38. The modified cell of embodiment 37, wherein the amino acid sequence of ECD of the first fusion protein subunit is SEQ ID NO: 63, and the amino acid sequence of ECD of the second fusion protein subunit is SEQ ID NO: 64.
39. The modified cell of embodiment 37 or embodiment 38, wherein the amino acid sequence of intracellular domain of the first fusion protein subunit is SEQ ID NO: 69, and the amino acid sequence of intracellular domain of the second fusion protein subunit is SEQ ID NO: 70.
40. The modified cell of any one of embodiments 37-39, wherein the first fusion protein subunit further comprises a transmembrane domain between the ECD and the intracellular domain thereof, and the second fusion protein subunit further comprises a transmembrane domain between the ECD and the intracellular domain thereof.
41. The modified cell of embodiment 40, wherein the transmembrane domain of the first fusion protein subunit is of the interleukin-2 receptor subunit gamma, and the transmembrane domain of the second fusion protein subunit is of the interleukin-2 receptor subunit beta.
42. The modified cell of embodiment 41, wherein the amino acid sequence of transmembrane domain of the first fusion protein subunit is SEQ ID NO: 67, and the amino acid sequence of transmembrane domain of the second fusion protein subunit is SEQ ID NO: 68.
43. The modified cell of any one of embodiments 37-42, wherein the first fusion protein subunit further comprises a membrane-proximal region between the ECD and the intracellular domain thereof, and the second fusion protein subunit further comprises a membrane-proximal region between the ECD and the intracellular domain thereof.
44. The modified cell of embodiment 43, wherein the membrane-proximal region of the first fusion protein subunit is of the interleukin-2 receptor subunit gamma, and the membrane-proximal region of the second fusion protein subunit is of the interleukin-2 receptor subunit beta.
45. The modified cell of embodiment 44, wherein the amino acid sequence of membrane-proximal region of the first fusion protein subunit is SEQ ID NO: 65, and the amino acid sequence of membrane-proximal region of the second fusion protein subunit is SEQ ID NO: 66.
46. The modified cell of any one of embodiment 37-45, wherein a first exogenous polynucleotide encoding the first fusion protein subunit of the signal converter is inserted into a SOCS1 locus.
47. The modified cell of any one of embodiments 37-46, wherein a second exogenous polynucleotide encoding the second fusion protein subunit of the signal converter is inserted into a ROSA26 locus.
48. The modified cell of any one of embodiments 37-47, the first fusion protein subunit of the signal converter comprises an amino acid sequence of SEQ ID NO: 71, and the second fusion protein subunit of the signal converter comprises an amino acid sequence of SEQ ID NO: 72; optionally the first exogenous polynucleotide encodes an amino acid sequence comprising SEQ ID NO: 75, and the second exogenous polynucleotide encodes an amino acid sequence comprising SEQ ID NO: 76.
49. The modified cell of any one of embodiments 37-48, wherein the amino acid sequence of the first fusion protein subunit is SEQ ID NO: 71, and the amino acid sequence of the second fusion protein subunit is SEQ ID NO: 72.
50. The modified cell of any one of embodiments 1-49, further comprising an exogenous polynucleotide encoding a B2M-HLA-E fusion protein (B2M-HLA-E) comprising at least a portion of β2M fused with at least a portion of HLA-E.
51. The modified cell of embodiment 50, wherein the exogenous polynucleotide encoding the B2M-HLA-E is inserted into a β2M locus.
52. The modified cell of embodiment 50 or embodiment 51, wherein the B2M-HLA-E comprises an amino acid sequence of SEQ ID NO: 81.
53. The modified cell of any one of embodiments 1-52, further comprising one or more exogenous polynucleotides encoding a TCR, or further comprising one or more exogenous polynucleotides encoding TCRαβ.
54. The modified cell of embodiment 53, wherein the one or more exogenous polynucleotides encoding the TCRαβ are inserted into a TRAC locus.
55. The modified cell of embodiment 53 or embodiment 54, wherein the TCRαβ specifically binds KRAS G12V.
56. The modified cell of embodiment 55, wherein the TCRαβ which specifically binds KRAS G12V comprises an α chain having the amino acid sequence of SEQ ID NO: 79 and a β chain having the amino acid sequence of SEQ ID NO: 80; or the TCRαβ which specifically binds KRAS G12V comprises an α chain having the amino acid sequence of SEQ ID NO: 82 and a β chain having the amino acid sequence of SEQ ID NO: 83.
57. The modified cell of any one of embodiments 1-56, further comprising one or more exogenous polynucleotides encoding a chimeric antigen receptor (CAR), or further comprising an exogenous polynucleotide encoding a chimeric antigen receptor (CAR) which specifically binds human CD19, or further comprising an exogenous polynucleotide encoding a chimeric antigen receptor (CAR) which specifically binds human CD20.
58. The modified cell of any one of embodiments 1-56, further comprising an exogenous polynucleotide encoding a chimeric antigen receptor (CAR) which specifically binds human CD19, and further comprising an exogenous polynucleotide encoding a chimeric antigen receptor (CAR) which specifically binds human CD20.
59. The modified cell of embodiment 57 or embodiment 58, wherein the exogenous polynucleotide encoding the CAR which specifically binds human CD19 is inserted into an AAVS1 locus.
60. The modified cell of any one of embodiments 57-59, wherein the CAR which specifically binds human CD19 comprises the amino acid sequence of SEQ ID NO: 93 or SEQ ID NO: 94.
61. The modified cell of any one of embodiments 1-60, further comprising an exogenous polynucleotide encoding a suicide switch, which comprises a herpes simplex virus thymidine kinase (HSV-tk) polypeptide or a functional variant thereof capable of inducing cell death upon administration of Ganciclovir (GCV).
62. The modified cell of embodiments 61, wherein the suicide switch comprises an amino acid sequence of SEQ ID NO: 85.
63. The modified cell of embodiments 61 or embodiment 62, wherein the suicide switch is inserted into a Rogi2 locus, or wherein the suicide switch is inserted into a FAS locus.
64. The modified cell of any one of embodiments 1-63, wherein the iPSC is derived from a primary T cell (T-iPSC).
65. The modified cell of any one of embodiments 1-64, wherein the iPSC is derived from a primary αβT cell (αβT-iPSC).
66. The modified cell of any one of embodiments 1-65, wherein the αβT cell is an iPSC-derived αβT (iαβT) cell.
67. The modified cell of any one of embodiments 1-66, wherein the αβT cell is a αβT cell derived from a T-iPSC (T-iαβT).
68. The modified cell of any one of embodiments 1-67, wherein the αβT cell is a αβT cell differentiated from a αβT-iPSC (αβT-iαβT).
69. The modified cell of any one of embodiments 1-68, wherein the intermediate cell differentiated from the iPSC is a αβT-iHSC, a αβT-iCLP, and/or an immature T-iαβT cell.
70. A population of cells comprising the modified cell of any one of embodiments 1-69.
71. The population of cells of embodiment 70, frozen in storage at about-196 degrees Celsius.
72. A method of generating a modified iPSC, αβT cell, or intermediate cell differentiated from an iPSC comprising contacting the cell with guide RNAs and an endonuclease or a nucleic acid encoding the endonuclease, wherein the contacting results in a genomic disruption in an endogenous SOCS1 gene that suppresses or eliminates expression of a functional SOCS1 protein and a genomic disruption in an endogenous CISH gene that suppresses or eliminates expression of a functional CISH protein.
73. The method of embodiment 72, wherein the iPSC is derived from a primary T cell (T-iPSC).
74. The method of embodiment 72 or embodiment 73, wherein the iPSC is derived from a primary αβT cell (αβT-iPSC).
75. The method of any one of embodiments 72-74, wherein the αβT cell is an iPSC-derived αβT (iαβT) cell.
76. The method of any one of embodiments 72-75, wherein the αβT cell is a αβT cell derived from a T-iPSC (T-iαβT).
77. The method of any one of embodiments 72-76, wherein the αβT cell is a αβT cell differentiated from a αβT-iPSC (αβT-iαβT).
78. The method of any one of embodiments 72-77, wherein the intermediate cell differentiated from the iPSC is a αβT-iHSC, a αβT-iCLP, and/or an immature T-iαβT cell.
79. The method of any one of embodiments 72-78, wherein the genomic disruption is in exon 2 of the SOCS1 gene; optionally wherein the genomic disruption is an inactivating nucleic acid mutation, optionally wherein the mutation is an insertion, deletion, or substitution.
80. The method of any one of embodiments 72-79, wherein the genomic disruption is in exon 3 or exon 4 of the CISH gene; optionally wherein the genomic disruption is an inactivating nucleic acid mutation, optionally wherein the mutation is an insertion, deletion, or substitution.
81. The method of any one of embodiments 72-80, wherein the genomic disruption is within a target sequence in SOCS1 that comprises SEQ ID NO: 17 or SEQ ID NO: 23 and/or the genomic disruption is within a target sequence in CISH that comprises SEQ ID NO: 15, SEQ ID NO: 16 or SEQ ID NO: 24.
82. The method of any one of embodiments 72-81, wherein the genomic disruption within SOCS1 and/or CISH is made by administering guide RNAs to said cell, and optionally wherein a Cas endonuclease or a nucleic acid encoding the Cas endonuclease is also administered to said cell, optionally wherein the endonuclease is a Cas9 endonuclease or wherein the endonuclease is a MAD7 endonuclease.
83. The method of embodiment 82, wherein the guide RNAs comprise the guide sequence of any one or more of SEQ ID NOs: 5, 11, 3, 4, and 12, or comprises the sgRNA or crRNA sequence of any one or more of SEQ ID NOs: 29, 37, 27, 28, and 38.
84. The method of embodiment 82 or embodiment 83, wherein (1) the endonuclease is the MAD7 endonuclease, and the guide RNAs comprise the guide sequence of SEQ ID NO: 11, or comprise the crRNA sequence of SEQ ID NO: 37; or (2) the endonuclease is the Cas9 endonuclease, and the guide RNAs comprise the guide sequence of SEQ ID NO: 5, or comprise the sgRNA sequence of SEQ ID NO: 29.
85. The method of embodiment 82 or embodiment 83, wherein (1) the endonuclease is the MAD7 endonuclease, and the guide RNAs comprise the guide sequence of SEQ ID NO: 12, or comprise the crRNA sequence of SEQ ID NO: 38; or (2) the endonuclease is the Cas9 endonuclease, and the guide RNAs comprise the guide sequence of any one of SEQ ID NOs: 3 and 4, or comprise the sgRNA sequence of any one of SEQ ID NOs: 27 and 28.
86. The method of any one of embodiments 72-85, further comprising inhibiting expression of an endogenous BIM gene by causing a genomic disruption in an endogenous BIM gene.
87. The method of embodiment 86, wherein the genomic disruption in the BIM gene is in exon 2C of the BIM gene.
88. The method of embodiment 86 or embodiment 87, wherein the expression and/or function of BIM_{EL} and BIM_{L} splice variants are reduced in the cell relative to that in an unmodified control and the expression and/or function of BIM_{S} splice variant is retained in the cell.
89. The method of any one of embodiments 86-88, wherein the genomic disruption is an inactivating nucleic acid mutation in an exon 2C of an endogenous BIM gene, optionally wherein the mutation is an insertion, deletion, or substitution.
90. The method of any one of embodiments 86-89, wherein the genomic disruption is within a target sequence in BIM that comprises any one of SEQ ID NOs: 14, 18, 19, 20, 21, and 22.
91. The method of any one of embodiments 86-90, wherein the genomic disruption within BIM is made by administering a guide RNA to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
92. The method of embodiment 91, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
93. The method of embodiment 91 or embodiment 92, wherein (1) the endonuclease is the MAD7 endonuclease, and the guide RNAs comprise the guide sequence of SEQ ID NO: 10, or comprise the crRNA sequence of SEQ ID NO: 36; or (2) the endonuclease is the Cas9 endonuclease, and the guide RNAs comprise the guide sequence of any one of SEQ ID NOs: 2, 6, 7, 8, and 9, or comprises the sgRNA sequence of any one of SEQ ID NOs: 26, 32, 33, 34, and 35.
94. The method of any one of embodiments 72-93, further comprising inhibiting expression of an endogenous FAS gene by causing a genomic disruption in an endogenous FAS gene that suppresses or eliminates expression of a functional FAS protein.
95. The method of embodiment 94, wherein the genomic disruption in the FAS gene is in exon 1 of the FAS gene.
96. The method of embodiment 94 or embodiment 95, wherein the genomic disruption is an inactivating nucleic acid mutation in exon 1 of an endogenous FAS gene, optionally wherein the mutation is an insertion, deletion, or substitution.
97. The method of any one of embodiments 94-96, wherein the genomic disruption is within a target sequence in FAS that comprises SEQ ID NO: 62.
98. The method of any one of embodiments 94-97, wherein the genomic disruption within FAS is made by administering a guide RNA to said cell, and optionally wherein an endonuclease or a nucleic acid encoding the endonuclease is also administered to said cell.
99. The method of embodiment 98, wherein the guide RNA comprises the guide sequence of SEQ ID NO: 61, or comprises the sgRNA sequence of SEQ ID NO: 60.
100. The method of any one of embodiments 72-99, further comprising inhibiting expression of an endogenous B2M gene by causing a genomic disruption in an endogenous B2M gene that suppresses or eliminates expression of a functional B2M protein.
101. The method of embodiment 100, wherein the genomic disruption in the B2M gene is in exon 2 of the B2M gene.
102. The method of embodiment 100 or embodiment 101, wherein the genomic disruption is an inactivating nucleic acid mutation in exon 2 of an endogenous B2M gene, optionally wherein the mutation is an insertion, deletion, or substitution.
103. The method of any one of embodiments 100-102, wherein the genomic disruption is within a target sequence in B2M that comprises SEQ ID NO: 56.
104. The method of any one of embodiments 100-103, wherein the genomic disruption within B2M is made by administering a guide RNA to said cell, and optionally wherein an endonuclease or a nucleic acid encoding the endonuclease is also administered to said cell.
105. The method of embodiment 104, wherein the guide RNA comprises the guide sequence of SEQ ID NO: 55, or comprises the sgRNA sequence of SEQ ID NO: 54.
106. The method of any one of embodiments 72-105, further comprising inhibiting expression of an endogenous CIITA gene by causing a genomic disruption in an endogenous CIITA gene that suppresses or eliminates expression of a functional CIITA protein.
107. The method of embodiment 106, wherein the genomic disruption in the CIITA gene is in exon 3 of the CIITA gene.
108. The method of embodiment 106 or embodiment 107, wherein the genomic disruption is an inactivating nucleic acid mutation in exon 3 of an endogenous CIITA gene, optionally wherein the mutation is an insertion, deletion, or substitution.
109. The method of any one of embodiments 106-108, wherein the genomic disruption is within a target sequence in CIITA that comprises SEQ ID NO: 59.
110. The method of any one of embodiments 106-109, wherein the genomic disruption within CIITA is made by administering a guide RNA to said cell, and optionally wherein an endonuclease or a nucleic acid encoding the endonuclease is also administered to said cell.
111. The method of embodiment 110, wherein the guide RNA comprises the guide sequence of SEQ ID NO: 58, or comprises the sgRNA sequence of SEQ ID NO: 57.
112. The method of any one of embodiments 72-111, further comprising inhibiting expression of an endogenous TRAC gene by causing a genomic disruption in an endogenous TRAC gene that suppresses or eliminates expression of a functional TRAC protein and/or inhibiting expression of an endogenous TRBC1/2 gene by causing a genomic disruption in an endogenous TRBC1/2 gene that suppresses or eliminates expression of a functional TRBC1/2 protein.
113. The method of any one of embodiments 72-112, wherein the genomic disruption is an inactivating nucleic acid mutation, optionally wherein the mutation is an insertion, deletion, or substitution.
114. The method of any one of embodiments 72-113, further comprising introducing one or more exogenous polynucleotides encoding a signal converter into the cell, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).
115. The method of embodiment 114, wherein a first exogenous polynucleotide encoding the first fusion protein subunit of the signal converter is inserted into a SOCS1 locus.
116. The method of embodiment 114 or 115, wherein a second exogenous polynucleotide encoding the second fusion protein subunit of the signal converter is inserted into a ROSA26 locus.
117. The method of any one of embodiments 114-116, the first fusion protein subunit of the signal converter comprises an amino acid sequence of SEQ ID NO: 71, and the second fusion protein subunit of the signal converter comprises an amino acid sequence of SEQ ID NO: 72; optionally the first exogenous polynucleotide encodes an amino acid sequence comprising SEQ ID NO: 75, and the second exogenous polynucleotide encodes an amino acid sequence comprising SEQ ID NO: 76.
118. The method of any one of embodiments 72-117, further comprising introducing an exogenous polynucleotide encoding a B2M-HLA-E fusion protein (B2M-HLA-E) comprising at least a portion of B2M fused with at least of a portion of HLA-E into the cell.
119. The method of embodiment 118, wherein the exogenous polynucleotide encoding the B2M-HLA-E is inserted into a B2M locus.
120. The method of any one of embodiments 118-119, wherein the B2M-HLA-E comprises an amino acid sequence of SEQ ID NO: 81.
121. The method of any one of embodiments 72-120, further comprising introducing one or more exogenous polynucleotides encoding TCRαβ into the cell.
122. The method of embodiment 121, wherein the one or more exogenous polynucleotides encoding the TCRαβ are inserted into a TRAC locus.
123. The method of embodiment 121 or embodiment 122, wherein the one or more exogenous polynucleotides encode the TCRαβ which specifically binds KRAS G12V.
124. The method of embodiment 123, wherein the TCRαβ which specifically binds KRAS G12V comprises an α chain having the amino acid sequence of SEQ ID NO: 79 and a β chain having the amino acid sequence of SEQ ID NO: 80; or the TCRαβ which specifically binds KRAS G12V comprises an α chain having the amino acid sequence of SEQ ID NO: 82 and a β chain having the amino acid sequence of SEQ ID NO: 83.
125. The method of any one of embodiments 72-124, further comprising introducing an exogenous polynucleotide encoding a chimeric antigen receptor (CAR) which specifically binds human CD19 or further comprising introducing an exogenous polynucleotide encoding a chimeric antigen receptor (CAR) which specifically binds human CD20.
126. The method of any one of embodiments 72-124, further comprising introducing an exogenous polynucleotide encoding a chimeric antigen receptor (CAR) which specifically binds human CD19, and further comprising introducing an exogenous polynucleotide encoding a chimeric antigen receptor (CAR) which specifically binds human CD20.
127. The method of embodiment 125 or embodiment 126, wherein the exogenous polynucleotide encoding the CAR which specifically binds human CD19 is inserted into an AAVS1 locus.
128. The method of any one of embodiments 125-127, wherein the CAR which specifically binds human CD19 comprises the amino acid sequence of SEQ ID NO: 93 or SEQ ID NO: 94.
129. The method of any one of embodiments 72-128, further comprising introducing an exogenous polynucleotide encoding a suicide switch, which comprises a herpes simplex virus thymidine kinase (HSV-tk) polypeptide or a functional variant thereof capable of inducing cell death upon administration of Ganciclovir (GCV).
130. The method of embodiments 129, wherein the suicide switch comprises an amino acid sequence of SEQ ID NO: 85.
131. The method of embodiments 129 or embodiment 130, wherein the suicide switch is inserted into a Rogi2 locus, or wherein the suicide switch is inserted into a FAS locus.
132. A modified iPSC, αβT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell.
133. The modified cell of embodiment 132, wherein the genomic disruption is an inactivating nucleic acid mutation in the exon 2C, optionally wherein the mutation is an insertion, deletion, or substitution.
134. The modified cell of embodiment 132 or embodiment 133, wherein the genomic disruption is within a target sequence in BIM that comprises any one of SEQ ID NOs: 14, 20, 21, and 22.
135. The modified cell of any one of embodiments 132-134, wherein the genomic disruption within BIM is made by administering a guide RNA to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
136. The modified cell of embodiment 135, wherein the guide RNA comprises the sgRNA or crRNA sequence of any one or more of SEQ ID NOs: 26, 34, 35 and 36, or the guide sequence differing by no more than 3 nucleotides from any one or more of SEQ ID Nos: 2, 8, 9 and 10.
137. The modified cell of embodiment 135 or embodiment 136, wherein the guide RNA comprises the crRNA sequence of SEQ ID NO: 36 or the guide sequence differing by no more than 3 nucleotides from SEQ ID NO: 10, and the endonuclease is a MAD7 endonuclease.
138. The modified cell of embodiment 135 or embodiment 136, wherein the guide RNA comprises the sgRNA sequence of any one of SEQ ID NOs: 26, 34, and 35, or the guide sequence differing by no more than 3 nucleotides from any one of SEQ ID Nos: 2, 8, and 9, and the endonuclease is a Cas9 endonuclease.
139. A method of inhibiting expression of an endogenous BIM gene in a iPSC, αβT cell, or intermediate cell differentiated from an iPSC, comprising contacting the cell with a guide RNA and an endonuclease or a nucleic acid encoding the endonuclease, wherein the contacting results in a genomic disruption in an exon 2C of an endogenous BIM gene, wherein the expression and/or function of BIM_{EL} and BIM_{L} splice variants are reduced in the cell relative to that in an unmodified control and the expression and/or function of BIM_{S} splice variant is retained in the cell.
140. The method of embodiment 139, wherein the genomic disruption is an inactivating nucleic acid mutation in the exon 2C, optionally wherein the mutation is an insertion, deletion, or substitution, and optionally wherein the endonuclease is a Cas9 endonuclease or wherein the endonuclease is a MAD7 endonuclease.
141. The method of embodiment 139 or embodiment 140, wherein the genomic disruption is within a target sequence in BIM that comprises any one of SEQ ID NOs: 14, 20, 21, and 22.
142. The method of any one of embodiments 139-141, wherein the guide RNA comprises the sgRNA or crRNA sequence of any one or more of SEQ ID NOs: 26, 34, 35 and 36, or the guide sequence differing by no more than 3 nucleotides from any one or more of SEQ ID Nos: 2, 8, 9 and 10.
143. The method of any one of embodiments 139-142, wherein (1) the guide RNA comprises the guide sequence of any one of SEQ ID NOs: 2, 8, and 9, or comprises the sgRNA sequence of any one of SEQ ID Nos: 26, 34, and 35, and the endonuclease is a Cas9 endonuclease; or (2) the guide RNA comprises the guide sequence of SEQ ID NO: 10, or comprises the crRNA sequence of SEQ ID NO: 36, and the endonuclease is a MAD7 endonuclease.
144. The method of any one of embodiments 139-143, wherein the αβT cell is a αβT cell differentiated from a αβT-iPSC (αβT-iαβT).
145. The method of any one of embodiments 139-143, wherein the cell is generated from the method of any one of embodiments 72-131.
146. A modified cell generated from the method of any one of embodiments 72-131 and embodiments 139-145.
147. A modified iPSC, αβT cell, or intermediate cell differentiated from an iPSC, comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).
148. The modified cell of embodiment 147, further comprising one or more exogenous polynucleotides encoding TCRαβ, optionally wherein the one or more exogenous polynucleotides encoding the TCRαβ is inserted into a TRAC locus, optionally wherein the TCRαβ specifically binds KRAS G12V, optionally wherein the TCRαβ comprises an α chain having the amino acid sequence of SEQ ID NO: 79 and a β chain having the amino acid sequence of SEQ ID NO: 80, or the TCRαβ comprises an α chain having the amino acid sequence of SEQ ID NO: 82 and a β chain having the amino acid sequence of SEQ ID NO: 83.
149. The modified cell of embodiment 147 or embodiment 148, further comprising an exogenous polynucleotide encoding a chimeric antigen receptor (CAR) which specifically binds human CD19 and/or an exogenous polynucleotide encoding a chimeric antigen receptor (CAR) which specifically binds human CD20, optionally wherein the exogenous polynucleotide encoding the CAR which specifically binds human CD19 is inserted into an AAVS1 locus, optionally wherein the CAR which specifically binds human CD19 comprises the amino acid sequence of SEQ ID NO: 93 or SEQ ID NO: 94.
150. The modified cell of any one of embodiments 147-149, wherein the amino acid sequence of ECD of the first fusion protein subunit is SEQ ID NO: 63, and the amino acid sequence of ECD of the second fusion protein subunit is SEQ ID NO: 64.
151. The modified cell of any one of embodiments 147-150, wherein the amino acid sequence of intracellular domain of the first fusion protein subunit is SEQ ID NO: 69, and the amino acid sequence of intracellular domain of the second fusion protein subunit is SEQ ID NO: 70.
152. The modified cell of any one of embodiments 147-151, wherein the first fusion protein subunit further comprises a transmembrane domain between the ECD and the intracellular domain thereof, and the second fusion protein subunit further comprises a transmembrane domain between the ECD and the intracellular domain thereof.
153. The modified cell of embodiment 152, wherein the transmembrane domain of the first fusion protein subunit is of the interleukin-2 receptor subunit gamma, and the transmembrane domain of the second fusion protein subunit is of the interleukin-2 receptor subunit beta.
154. The modified cell of embodiment 153, wherein the amino acid sequence of transmembrane domain of the first fusion protein subunit is SEQ ID NO: 67, and the amino acid sequence of transmembrane domain of the second fusion protein subunit is SEQ ID NO: 68.
155. The modified cell of any one of embodiments 147-154, wherein the first fusion protein subunit further comprises a membrane-proximal region between the ECD and the intracellular domain thereof, and the second fusion protein subunit further comprises a membrane-proximal region between the ECD and the intracellular domain thereof.
156. The modified cell of embodiment 155, wherein the membrane-proximal region of the first fusion protein subunit is of the interleukin-2 receptor subunit gamma, and the membrane-proximal region of the second fusion protein subunit is of the interleukin-2 receptor subunit beta.
157. The modified cell of embodiment 156, wherein the amino acid sequence of membrane-proximal region of the first fusion protein subunit is SEQ ID NO: 65, and the amino acid sequence of membrane-proximal region of the second fusion protein subunit is SEQ ID NO: 66.
158. The modified cell of any one of embodiment 147-157, wherein a first exogenous polynucleotide encoding the first fusion protein subunit of the signal converter is inserted into a SOCS1 locus.
159. The modified cell of any one of embodiments 147-158, wherein a second exogenous polynucleotide encoding the second fusion protein subunit of the signal converter is inserted into a ROSA26 locus.
160. The modified cell of any one of embodiments 147-159, the first fusion protein subunit of the signal converter comprises an amino acid sequence of SEQ ID NO: 71, and the second fusion protein subunit of the signal converter comprises an amino acid sequence of SEQ ID NO: 72; optionally the first exogenous polynucleotide encodes an amino acid sequence comprising SEQ ID NO: 75, and the second exogenous polynucleotide encodes an amino acid sequence comprising SEQ ID NO: 76.
161. The modified cell of any one of embodiments 147-160, wherein the amino acid sequence of the first fusion protein subunit is SEQ ID NO: 71, and the amino acid sequence of the second fusion protein subunit is SEQ ID NO: 72.
162. The modified cell or method of any one of the preceding embodiments, wherein the cell is a human cell.
163. The modified cell or method of any one of the preceding embodiments, wherein the cell is in vivo, in vitro, ex vivo, or in a human subject.
164. A gene editing system comprising one or more guide RNA and an endonuclease or a nucleic acid encoding the endonuclease, wherein the one or more guide RNAs comprise one or more single guide RNA (sgRNA) or CRISPR RNA (crRNA) that comprises:
a. a guide sequence that binds to a target sequence comprising any one of SEQ ID NOs: 17, 23, 15, 16, and 24;
b. a guide sequence that binds to a target sequence comprising any one of SEQ ID NOs: 14, 18, 19, 20, 21, and 22;
c. a first guide sequence that binds to a target sequence comprising any one of SEQ ID NOs: 17 and 23; and a second guide sequence that binds to a target sequence comprising any one of SEQ ID NOs: 15, 16, and 24;
d. a first guide sequence that binds to a target sequence comprising any one of SEQ ID NOs: 17 and 23; a second guide sequence that binds to a target sequence comprising any one of SEQ ID NOs: 15, 16, and 24; and a third guide sequence that binds to a target sequence comprising any one of SEQ ID NOs: 14, 18, 19, 20, 21, and 22;
e. a first guide sequence that binds to a target sequence comprising any one of SEQ ID NOs: 17 and 23; a second guide sequence that binds to a target sequence comprising any one of SEQ ID NOs: 15, 16, and 24; a third guide sequence that binds to a target sequence comprising any one of SEQ ID NOs: 14, 18, 19, 20, 21, and 22; a fourth guide sequence that binds to a target sequence comprising SEQ ID NO: 56; and, a fifth guide sequence that binds to a target sequence comprising SEQ ID NO: 59;
f. a first guide sequence that binds to a target sequence comprising any one of SEQ ID NOs: 17 and 23; a second guide sequence that binds to a target sequence comprising any one of SEQ ID NOs: 15, 16, and 24; a third guide sequence that binds to a target sequence comprising any one of SEQ ID NOs: 14, 18, 19, 20, 21, and 22; a sixth guide sequence that binds to a target sequence comprising SEQ ID NO: 62; a fourth guide sequence that binds to a target sequence comprising SEQ ID NO: 56; and a fifth guide sequence that binds to a target sequence comprising SEQ ID NO: 59;
g. a guide sequence that comprises any one of SEQ ID NOs: 5 and 11;
h. a sgRNA or crRNA sequence that comprises SEQ ID NO: 29 and 37;
i. a guide sequence that comprises any one of SEQ ID NOs: 3, 4, and 12;
j. asgRNA or crRNA sequence that comprises any one of SEQ ID NOs: 27, 28, and 38;
k. a guide sequence that comprises any one of SEQ ID NOs: 2, 6, 7, 8, 9, 10;
l. a sgRNA or crRNA sequence that comprises any one of SEQ ID NOs: 26, 32, 33, 34, 35, and 36;
m. a first guide sequence that comprises any one of SEQ ID NOs: 5 and 11, and a second guide sequence that comprises any one of SEQ ID NOs: 3, 4, and 12;
n. a first sgRNA or crRNA sequence that comprises any one of SEQ ID NOs: 29 and 37, and a second sgRNA or crRNA sequence that comprises any one of SEQ ID NOs: 27, 28, and 38;
o. a first guide sequence that comprises any one of SEQ ID NOs: 5 and 11; a second guide sequence that comprises any one of SEQ ID NOs: 3, 4, and 12; and a third guide sequence that comprises any one of SEQ ID NOs: 2, 6, 7, 8, 9, and 10;
p. a first sgRNA or crRNA sequence that comprises any one of SEQ ID NOs: 29 and 37; a second sgRNA or crRNA sequence that comprises any one of SEQ ID NOs: 27, 28, and 38; and a third sgRNA or crRNA sequence that comprises any one of SEQ ID NOs: 26, 32, 33, 34, 35, and 36;
q. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a fourth guide sequence that comprises SEQ ID NO: 55; and, a fifth guide sequence that comprises SEQ ID NO: 58;
r. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a fourth sgRNA sequence that comprises SEQ ID NO: 54; and, a fifth sgRNA sequence that comprises SEQ ID NO: 57;
s. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a sixth guide sequence that comprises SEQ ID NO: 61; a fourth guide sequence that comprises SEQ ID NO: 55; and, a fifth guide sequence that comprises SEQ ID NO: 58; or
t. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a sixth sgRNA sequence that comprises SEQ ID NO: 60; a fourth sgRNA sequence comprises SEQ ID NO: 54; and, a fifth sgRNA sequence that comprises SEQ ID NO: 57.
   165. The gene editing system of embodiment 164, wherein the endonuclease is a Cas endonuclease.
   166. The gene editing system of embodiment 165, wherein the Cas endonuclease is a MAD7 endonuclease.
   167. The gene editing system of embodiment 166, wherein:
a. the target sequence comprises SEQ ID NO: 22; and/or
b. the crRNA comprises a guide sequence differing by no more than 3 nucleotides from SEQ ID NO: 10.
   168. The gene editing system of embodiment 166, wherein:
a. the target sequence comprises SEQ ID NO: 23; and/or
b. the crRNA comprises a guide sequence differing by no more than 3 nucleotides from SEQ ID NO: 11.
   169. The gene editing system of embodiment 166, wherein:
a. the target sequence comprises SEQ ID NO: 24; and/or
b. the crRNA comprises a guide sequence differing by no more than 3 nucleotides from SEQ ID NO: 12.
   170. The gene editing system of embodiment 165, wherein the Cas endonuclease is a Cas9 endonuclease.
   171. The gene editing system of embodiment 170, wherein:
a. the target sequence comprises any one of SEQ ID NOs: 14, 18, 19, 20, and 21; and/or
b. the sgRNA comprises a guide sequence differing by no more than 3 nucleotides from any one of SEQ ID NOs: 2, 6, 7, 8, and 9.
   172. The gene editing system of embodiment 170, wherein:
a. the target sequence comprises SEQ ID NO: 17; and/or
b. the sgRNA comprises a guide sequence differing by no more than 3 nucleotides from SEQ ID NO: 5.
   173. The gene editing system of embodiment 170, wherein:
a. the target sequence comprises SEQ ID NOs: 15 or 16; and/or
b. the sgRNA comprises a guide sequence differing by no more than 3 nucleotides from SEQ ID NOs: 3 or 4.
   174. The gene editing system of embodiment 170, wherein:
a. the target sequence comprises SEQ ID NO: 62; and/or
b. the sgRNA comprises a guide sequence differing by no more than 3 nucleotides from SEQ ID NO: 61.
   175. The gene editing system of embodiment 170, wherein:
a. the target sequence comprises SEQ ID NO: 56; and/or
b. the sgRNA comprises a guide sequence differing by no more than 3 nucleotides from SEQ ID NO: 55.
   176. The gene editing system of embodiment 170, wherein:
a. the target sequence comprises SEQ ID NO: 59; and/or
b. the sgRNA comprises a guide sequence differing by no more than 3 nucleotides from SEQ ID NO: 58.
   177. A guide RNA comprising SEQ ID NO: 5 (SOCS1), SEQ ID NO: 29 (SOCS1), SEQ ID NO: 11 (SOCS1), SEQ ID NO: 37 (SOCS1), SEQ ID NO: 27 (CISH), SEQ ID NO: 28 (CISH), SEQ ID NO: 12 (CISH), SEQ ID NO: 38 (CISH), SEQ ID NO: 6 (BIM), SEQ ID NO: 32 (BIM), SEQ ID NO: 7 (BIM), SEQ ID NO: 33 (BIM), SEQ ID NO: 8 (BIM), SEQ ID NO: 34 (BIM), SEQ ID NO: 9 (BIM), SEQ ID NO: 35 (BIM), SEQ ID NO: 10 (BIM), or SEQ ID NO: 36 (BIM).
   178. A guide RNA comprising a CRISPR RNA (crRNA) sequence, wherein the crRNA sequence comprises a 2′-O-Methyl modification at each of the first three positions on the 5′ end and at each of the fourth to last, third to last, and second to last positions of the crRNA sequence on the 3′ end, and a phosphorothioate linkage between each base in the first three positions on the 5′ end and a phosphorothioate linkage between each base of the last three positions of the crRNA sequence at the 3′ end.
   179. The guide RNA of embodiment 178, wherein the guide RNA functions with a MAD7 endonuclease.
   180. The guide RNA of any one of embodiments 178-179, wherein the crRNA is about 56 bases long.
   181. The guide RNA of any one of embodiments 178-180, wherein the crRNA sequence comprises a 2′-O-Methyl modification at each of positions 1, 2, 3, 53, 54, and 55 of the crRNA sequence on the 3′ end.
   182. The guide RNA of any one of embodiments 178-181, wherein the crRNA comprises SEQ ID NO: 36, SEQ ID NO: 37, or SEQ ID NO: 38, including its modifications.
   183. The guide RNA of any one of embodiments 178-182, wherein the last base of the crRNA is modified, but not with 2′-O-Methyl modification.
   184. The guide RNA of any one of embodiments 178-183, wherein the modification on the last base of the crRNA is a pseudoknot.
   185. The guide RNA of any one of embodiments 178-184, wherein the modification on the last base of the crRNA is a modification that facilitates use with MAD7.
   186. A gene editing system comprising one or more guide RNAs and an endonuclease or a nucleic acid encoding the endonuclease, wherein the one or more guide RNAs comprise one or more guide sequences that binds to a target sequence in an exon 2C of an endogenous BIM gene, wherein upon administering the gene editing system to a cell, the expression and/or function of BIM_{EL} and BIM_{L} splice variants are reduced in the cell relative to that in an unmodified control, and the expression and/or function of BIM_{S} splice variant is retained, optionally the targe sequence comprises any one of SEQ ID NOs: 14, 20, 21, and 22.
   187. The gene editing system of embodiment 186, wherein (1) the endonuclease is a MAD7 endonuclease, and the one or more guide RNAs comprise the crRNA sequence of SEQ ID NO: 36 or the guide sequence differing by no more than 3 nucleotides from SEQ ID NO: 10; or (2) the endonuclease is a Cas9 endonuclease, and the one or more guide RNAs comprise the sgRNA sequence of any one of SEQ ID NOs: 26, 34, and 35, or the guide sequence differing by no more than 3 nucleotides from any one of SEQ ID NOs: 2, 8, and 9.
   188. A gene editing system comprising one or more guide RNAs and an endonuclease or a nucleic acid encoding the endonuclease, wherein the one or more guide RNAs comprise one or more guide sequences which binds to a target sequence in CISH gene that comprises SEQ ID NO: 24; wherein the endonuclease is a MAD7 endonuclease, and the one or more guide RNAs comprise the crRNA sequence of SEQ ID NO: 38 or the guide sequence differing by no more than 3 nucleotides from SEQ ID NO: 12.
   189. A gene editing system comprising one or more guide RNA and an endonuclease or a nucleic acid encoding the endonuclease, wherein (1) the one or more guide RNAs comprise one or more guide sequences which binds to a target sequence in SOCS1 gene that comprises SEQ ID NO: 23; wherein the endonuclease is a MAD7 endonuclease, and the one or more guide RNAs comprise the crRNA sequence of SEQ ID NO: 37 or the guide sequence differing by no more than 3 nucleotides from SEQ ID NO: 11; or (2) the one or more guide RNAs comprise one or more guide sequences which binds to a target sequence in SOCS1 gene that comprises SEQ ID NO: 17, the endonuclease is a Cas9 endonuclease, and the one or more guide RNAs comprise the sgRNA sequence of SEQ ID NO: 29, or the guide sequence differing by no more than 3 nucleotides from SEQ ID NO: 5.
   190. A modified iPSC, αβT cell, or intermediate cell differentiated from an iPSC, generated by any of the following:
i) contacting the cell with the gene editing system of any one of embodiments 164-176 and 186-189, wherein the contacting results in a genomic disruption in a target sequence that binds to the single guide RNA (sgRNA) of the gene editing system; or
ii) contacting the cell with the guide RNA of any one of embodiments 177-185 and an endonuclease or a nucleic acid encoding the endonuclease, wherein the contacting results in a genomic disruption in a target sequence that binds to the guide RNA of embodiment 177; and wherein the genomic disruption is an inactivating nucleic acid mutation, optionally wherein the mutation is an insertion, deletion, or substitution.
   191. A modified iPSC, αβT cell, or intermediate cell differentiated from an iPSC, comprising a genomic disruption in a target sequence of an endogenous SOCS1 gene that suppresses or eliminates expression of a functional SOCS1 protein, wherein the target sequence comprises SEQ ID NO: 17 or SEQ ID NO: 23.
   192. The modified cell of embodiment 191, wherein the genomic disruption is an inactivating nucleic acid mutation, optionally wherein the mutation is an insertion, deletion, or substitution.
   193. The modified cell of embodiment 191 or embodiment 192, wherein the genomic disruption within SOCS1 is made by administering a guide RNA to said cell, and optionally wherein an endonuclease or a nucleic acid encoding the endonuclease is also administered to said cell; optionally wherein the endonuclease is a Cas9 endonuclease or wherein the endonuclease is a MAD7 endonuclease.
   194. The modified cell of embodiment 193, wherein the guide RNA comprises the sgRNA or crRNA sequence of any one of SEQ ID NOs: 29 and 37, or comprises the guide sequence differing by no more than 3 nucleotides from SEQ ID NO: 5 or 11.
   195. The modified cell of embodiment 193 or embodiment 194, wherein the guide RNA comprises the crRNA sequence of SEQ ID NO: 37 or comprises the guide sequence differing by no more than 3 nucleotides from SEQ ID NO: 11, and the endonuclease is a MAD7 endonuclease.
   196. The modified cell of embodiment 193 or embodiment 194, wherein the guide RNA comprises the sgRNA sequence of SEQ ID NO: 29 or comprises the guide sequence differing by no more than 3 nucleotides from SEQ ID NO: 5, and the endonuclease is a Cas9 endonuclease.
   197. A modified iPSC, αβT cell, or intermediate cell differentiated from an iPSC, comprising a genomic disruption in a target sequence of an endogenous CISH gene that suppresses or eliminates expression of CISH, wherein the target sequence comprises SEQ ID NO: 24.
   198. The modified cell of embodiment 197, wherein the genomic disruption is an inactivating nucleic acid mutation, optionally wherein the mutation is an insertion, deletion, or substitution.
   199. The modified cell of embodiment 197 or embodiment 198, wherein the genomic disruption within CISH is made by administering a guide RNA to said cell, and optionally wherein an endonuclease or a nucleic acid encoding the endonuclease is also administered to said cell; optionally wherein the endonuclease is a Cas9 endonuclease or wherein the endonuclease is a MAD7 endonuclease.
   200. The modified cell of embodiment 199, wherein the guide RNA comprises the crRNA sequence of SEQ ID NO: 38 or comprises the guide sequence differing by no more than 3 nucleotides from SEQ ID NO: 12.
   201. The modified cell of embodiment 199 or embodiment 200, wherein the guide RNA comprises the crRNA sequence of SEQ ID NO: 38 or comprises the guide sequence differing by no more than 3 nucleotides from SEQ ID NO: 12, and the endonuclease is a MAD7 endonuclease.
   202. The modified cell of any one of embodiments 191-201, wherein the iPSC is derived from a primary T cell (T-iPSC).
   203. The modified cell of any one of embodiments 191-202, wherein the iPSC is derived from a primary αβT cell (αβT-iPSC).
   204. The modified cell of any one of embodiments 191-203, wherein the αβT cell is an iPSC-derived αβT (iαβT) cell.
   205. The modified cell of any one of embodiments 191-204, wherein the αβT cell is a αβT cell differentiated from a T-iPSC (T-iαβT).
   206. The modified cell of any one of embodiments 191-205, wherein the αβT cell is an αβT cell differentiated from a αβT-iPSC (αβT-iαβT).
   207. The modified cell of any one of embodiments 191-206, wherein the intermediate cell differentiated from the iPSC is a αβT-iHSC, a αβT-iCLP, and/or an immature T-iαβT cell.
   208. The modified cell of any one of embodiments 191-207, wherein the cell is a human cell.
   209. The modified cell any one of embodiments 191-208, wherein the cell is in vivo, in vitro, ex vivo, or in a human subject.
   210. A modified iPSC, αβT cell, or intermediate cell differentiated from an iPSC, comprising 1) an exogenous polynucleotide encoding a chimeric antigen receptor (CAR) which specifically binds human CD19 and/or an exogenous polynucleotide encoding a chimeric antigen receptor (CAR) which specifically binds human CD20, 2) a genomic disruption in an endogenous suppressor of cytokine signaling 1 (SOCS1) gene that suppresses or eliminates expression of a functional SOCS1 protein, and 3) a genomic disruption in an endogenous cytokine-inducible SH2-containing protein (CISH) gene that suppresses or eliminates expression of a functional CISH protein.
   211. The modified cell of embodiment 210, further comprising a genomic disruption in an endogenous Bcl-2 Interacting Mediator of cell death (BIM) gene.
   212. The modified cell of embodiment 211, wherein the genomic disruption in the endogenous BIM gene is in exon 2C of the BIM gene.
   213. The modified cell of embodiment 212, wherein the expression and/or function of BIM_{EL} and BIM_{L} splice variants are reduced in the modified cell relative to that in an unmodified control and the expression and/or function of BIM_{S} splice variant is retained in the modified cell.
   214. The modified cell of any one of embodiments 211-213, wherein the genomic disruption in the endogenous BIM gene is an inactivating nucleic acid mutation in exon 2C of the endogenous BIM gene, optionally wherein the mutation is an insertion, deletion, or substitution.
   215. The modified cell of any one of embodiments 211-214, wherein the genomic disruption in the endogenous BIM gene is within a target sequence in BIM that comprises any one of SEQ ID NOs: 14, 18, 19, 20, 21, and 22.
   216. The modified cell of any one of embodiments 211-215, wherein the genomic disruption within BIM is made by administering a guide RNA to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
   217. The modified cell of embodiment 216, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
   218. The modified cell of embodiment 216 or embodiment 217, wherein (1) the endonuclease is the MAD7 endonuclease, and the guide RNAs comprise the guide sequence of SEQ ID NO: 10, or comprise the crRNA sequence of SEQ ID NO: 36; or (2) the endonuclease is the Cas9 endonuclease, and the guide RNAs comprise the guide sequence of any one of SEQ ID NOs: 2, 6, 7, 8, and 9, or comprises the sgRNA sequence of any one of SEQ ID NOs: 26, 32, 33, 34, and 35.
   219. A modified iPSC, αβT cell, or intermediate cell differentiated from an iPSC, comprising 1) an exogenous polynucleotide encoding a chimeric antigen receptor (CAR) which specifically binds human CD19 and/or an exogenous polynucleotide encoding a chimeric antigen receptor (CAR) which specifically binds human CD20, 2) a genomic disruption in an endogenous suppressor of cytokine signaling 1 (SOCS1) gene that suppresses or eliminates expression of a functional SOCS1 protein, 3) a genomic disruption in an endogenous cytokine-inducible SH2-containing protein (CISH) gene that suppresses or eliminates expression of a functional CISH protein, and 4) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell.
   220. The modified cell of embodiment 219, wherein the genomic disruption in the endogenous BIM gene is an inactivating nucleic acid mutation in the exon 2C of the BIM gene, optionally wherein the mutation is an insertion, deletion, or substitution.
   221. The modified cell of embodiment 219 or embodiment 220, wherein the genomic disruption in the endogenous BIM gene is within a target sequence in BIM that comprises any one of SEQ ID NOs: 14, 20, 21, and 22.
   222. The modified cell of any one of embodiments 219-221, wherein the genomic disruption within BIM is made by administering a guide RNA to said cell, and optionally wherein an endonuclease or a nucleic acid encoding the endonuclease is also administered to said cell, optionally wherein the endonuclease is a Cas9 endonuclease or wherein the endonuclease is a MAD7 endonuclease.
   223. The modified cell of embodiment 222, wherein the guide RNA comprises the sgRNA or crRNA sequence of any one of SEQ ID NOs: 26, 34, 35, and 36, or comprises the guide sequence of any one of SEQ ID Nos: 2, 8, 9 and 10.
   224. The modified cell of embodiment 222 or embodiment 223, wherein the guide RNA comprises the crRNA sequence of SEQ ID NO: 36 or comprises the guide sequence of SEQ ID NO: 10, and the endonuclease is a MAD7 endonuclease.
   225. The modified cell of embodiment 222 or embodiment 223, wherein the guide RNA comprises the sgRNA sequence of any one of SEQ ID NOs: 26, 34, and 35, or comprises the guide sequence of any one of SEQ ID Nos: 2, 8, and 9, and the endonuclease is a Cas9 endonuclease.
   226. The modified cell of any one of embodiments 210-225, wherein the exogenous polynucleotide encoding the CAR which specifically binds human CD19 is inserted into an AAVS1 locus.
   227. The modified cell of any one of embodiments 210-226, wherein the CAR which specifically binds human CD19 comprises the amino acid sequence of SEQ ID NO: 93 or SEQ ID NO: 94.
   228. The modified cell of any one of embodiments 210-227, further comprising one or more exogenous polynucleotides encoding TCRαβ.
   229. The modified cell of embodiment 228, wherein the one or more exogenous polynucleotides encoding the TCRαβ is inserted into a TRAC locus.
   230. The modified cell of any one of embodiments 228-229, wherein the TCRαβ specifically binds KRAS G12V; and optionally wherein the TCRαβ comprises an α chain having the amino acid sequence of SEQ ID NO: 79 and a β chain having the amino acid sequence of SEQ ID NO: 80; or optionally wherein the TCRαβ comprises an α chain having the amino acid sequence of SEQ ID NO: 82 and a β chain having the amino acid sequence of SEQ ID NO: 83.
   231. The modified cell of any one of embodiments 210-230, wherein the genomic disruption in the endogenous SOCS1 gene is in exon 2 of the SOCS1 gene; optionally wherein the genomic disruption is an inactivating nucleic acid mutation, optionally wherein the mutation is an insertion, deletion, or substitution.
   232. The modified cell of any one of embodiments 210-231, wherein the genomic disruption in the endogenous CISH gene is in exon 3 or exon 4 of the CISH gene; optionally wherein the genomic disruption is an inactivating nucleic acid mutation, optionally wherein the mutation is an insertion, deletion, or substitution.
   233. The modified cell of any one of embodiments 210-232, wherein the genomic disruption in the endogenous SOCS1 gene is within a target sequence in SOCS1 that comprises SEQ ID NO: 17 or SEQ ID NO: 23 and/or the genomic disruption in the endogenous CISH gene is within a target sequence in CISH that comprises SEQ ID NO: 15, SEQ ID NO: 16 or SEQ ID NO: 24.
   234. The modified cell of any one of embodiments 210-233, wherein the genomic disruption within SOCS1 and/or CISH is made by administering guide RNAs to said cell, and wherein an endonuclease or a nucleic acid encoding the endonuclease is also administered to said cell, optionally wherein the endonuclease is a Cas9 endonuclease or wherein the endonuclease is a MAD7 endonuclease.
   235. The modified cell of embodiment 234, wherein the guide RNAs comprise the sgRNA or crRNA sequence of any one or more of SEQ ID NOs: 29, 37, 27, 28, and 38, or comprises the guide sequence of any one or more of SEQ ID NOs: 5, 11, 3, 4, and 12.
   236. The modified cell of embodiment 234 or embodiment 235, wherein (1) the endonuclease is the MAD7 endonuclease, and the guide RNAs comprise the guide sequence of SEQ ID NO: 11, or comprise the crRNA sequence of SEQ ID NO: 37; or (2) the endonuclease is the Cas9 endonuclease, and the guide RNAs comprise the guide sequence of SEQ ID NO: 5, or comprise the sgRNA sequence of SEQ ID NO: 29.
   237. The modified cell of embodiment 234 or embodiment 235, wherein (1) the endonuclease is the MAD7 endonuclease, and the guide RNAs comprise the guide sequence of SEQ ID NO: 12, or comprise the crRNA sequence of SEQ ID NO: 38; or (2) the endonuclease is the Cas9 endonuclease, and the guide RNAs comprise the guide sequence of any one of SEQ ID NOs: 3 and 4, or comprise the sgRNA sequence of any one of SEQ ID NOs: 27 and 28.
   238. The modified cell of any one of embodiments 210-237, further comprising a genomic disruption in an endogenous FAS cell surface death receptor (FAS) gene that suppresses or eliminates expression of a functional FAS protein.
   239. The modified cell of embodiment 238, wherein the genomic disruption in the FAS gene is in exon 1 of the FAS gene.
   240. The modified cell of embodiment 238 or embodiment 239, wherein the genomic disruption is an inactivating nucleic acid mutation in exon 1 of an endogenous FAS gene, optionally wherein the mutation is an insertion, deletion, or substitution.
   241. The modified cell of any one of embodiments 238-240, wherein the genomic disruption is within a target sequence in FAS that comprises SEQ ID NO: 62.
   242. The modified cell of any one of embodiments 238-241, wherein the genomic disruption within FAS is made by administering a guide RNA to said cell, and optionally wherein an endonuclease or a nucleic acid encoding the endonuclease is also administered to said cell.
   243. The modified cell of embodiment 242, wherein the guide RNA comprises SEQ ID NO: 60 or comprises the guide sequence of SEQ ID NO: 61.
   244. The modified cell of any one of embodiments 210-243, further comprising a genomic disruption in an endogenous β-2-Microglobulin (B2M) gene that suppresses or eliminates expression of a functional B2M protein.
   245. The modified cell of embodiment 244, wherein the genomic disruption in the β2M gene is in exon 2 of the β2M gene.
   246. The modified cell of embodiment 244 or embodiment 245, wherein the genomic disruption is an inactivating nucleic acid mutation in exon 2 of an endogenous β2M gene, optionally wherein the mutation is an insertion, deletion, or substitution.
   247. The modified cell of any one of embodiments 244-246, wherein the genomic disruption is within a target sequence in β2M that comprises SEQ ID NO: 56.
   248. The modified cell of any one of embodiments 244-247, wherein the genomic disruption within β2M is made by administering a guide RNA to said cell, and optionally wherein an endonuclease or a nucleic acid encoding the endonuclease is also administered to said cell.
   249. The modified cell of embodiment 248, wherein the guide RNA comprises SEQ ID NO: 54 or comprises the guide sequence of SEQ ID NO: 55.
   250. The modified cell of any one of embodiments 210-249, further comprising a genomic disruption in an endogenous Class II transactivator (CIITA) gene that suppresses or eliminates expression of a functional CIITA protein.
   251. The modified cell of embodiment 250, wherein the genomic disruption in the CIITA gene is in exon 3 of the CIITA gene.
   252. The modified cell of embodiment 250 or embodiment 251, wherein the genomic disruption is an inactivating nucleic acid mutation in exon 3 of an endogenous CIITA gene, optionally wherein the mutation is an insertion, deletion, or substitution.
   253. The modified cell of any one of embodiments 250-252, wherein the genomic disruption is within a target sequence in CIITA that comprises SEQ ID NO: 59.
   254. The modified cell of any one of embodiments 250-253, wherein the genomic disruption within CIITA is made by administering a guide RNA to said cell, and optionally wherein an endonuclease or a nucleic acid encoding the endonuclease is also administered to said cell.
   255. The modified cell of embodiment 254, wherein the guide RNA comprises SEQ ID NO: 57 or comprises the guide sequence of SEQ ID NO: 58.
   256. The modified cell of any one of embodiments 210-255, further comprising a genomic disruption in an endogenous T-cell receptor alpha constant region (TRAC) gene that suppresses or eliminates expression of a functional TRAC protein and/or a genomic disruption in an endogenous TRBC1/2 gene that suppresses or eliminates expression of a functional TRBC1/2 protein.
   257. The modified cell of any one of embodiments 210-256, wherein the genomic disruption is an inactivating nucleic acid mutation, optionally wherein the mutation is an insertion, deletion, or substitution.
   258. The modified cell of any one of embodiments 210-257, further comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).
   259. The modified cell of embodiment 258, wherein a first exogenous polynucleotide encoding the first fusion protein subunit of the signal converter is inserted into a SOCS1 locus.
   260. The modified cell of embodiment 258 or embodiment 259, wherein a second exogenous polynucleotide encoding the second fusion protein subunit of the signal converter is inserted into a ROSA26 locus.
   261. The modified cell of any one of embodiments 258-260, the first fusion protein subunit of the signal converter comprises an amino acid sequence of SEQ ID NO: 71, and the second fusion protein subunit of the signal converter comprises an amino acid sequence of SEQ ID NO: 72; optionally the first exogenous polynucleotide encodes an amino acid sequence comprising SEQ ID NO: 75, and the second exogenous polynucleotide encodes an amino acid sequence comprising SEQ ID NO: 76.
   262. The modified cell of any one of embodiments 258-261, wherein the amino acid sequence of the first fusion protein subunit is SEQ ID NO: 71, and the amino acid sequence of the second fusion protein subunit is SEQ ID NO: 72.
   263. The modified cell of any one of embodiments 210-262, further comprising an exogenous polynucleotide encoding a B2M-HLA-E fusion protein (B2M-HLA-E) comprising at least a portion of B2M fused with at least a portion of HLA-E.
   264. The modified cell of embodiment 263, wherein the exogenous polynucleotide encoding the B2M-HLA-E is inserted into a B2M locus.
   265. The modified cell of embodiment 263 or embodiment 264, wherein the B2M-HLA-E comprises an amino acid sequence of SEQ ID NO: 81.
   266. The modified cell of any one of embodiments 210-265, further comprising an exogenous polynucleotide encoding a suicide switch, which comprises a herpes simplex virus thymidine kinase (HSV-tk) polypeptide or a functional variant thereof capable of inducing cell death upon administration of Ganciclovir (GCV).
   267. The modified cell of embodiments 266, wherein the suicide switch comprises an amino acid sequence of SEQ ID NO: 85.
   268. The modified cell of embodiments 266 or embodiment 267, wherein the suicide switch is inserted into a Rogi2 locus, or wherein the suicide switch is inserted into a FAS locus.
   269. The modified cell of any one of embodiments 210-268, wherein the iPSC is derived from a primary T cell (T-iPSC).
   270. The modified cell of any one of embodiments 210-269, wherein the iPSC is derived from a primary αβT cell (αβT-iPSC).
   271. The modified cell of any one of embodiments 210-270, wherein the αβT cell is an iPSC-derived αβT (iαβT) cell.
   272. The modified cell of any one of embodiments 210-271, wherein the αβT cell is a αβT cell derived from a T-iPSC (T-iαβT).
   273. The modified cell of any one of embodiments 210-272, wherein the αβT cell is a αβT cell differentiated from a αβT-iPSC (αβT-iαβT).
   274. The modified cell of any one of embodiments 210-273, wherein the intermediate cell differentiated from the iPSC is a αβT-iHSC, a αβT-iCLP, and/or an immature T-iαβT cell.
   275. A pharmaceutical composition comprising the modified αβT cell of any one of embodiments 210-274 and a pharmaceutically acceptable carrier.
   276. A method of killing B cells which are CD19 positive and/or CD20 positive and have abnormal B cell function, comprising administering to a subject in need thereof a therapeutically effective amount of the modified αβT cell of any one of embodiments 210-274 or the pharmaceutical composition of embodiment 275.
   277. The method of embodiment 276, wherein the B cells are the CD19 positive B cells in B-cell Acute Lymphoblastic Leukemia (B-ALL) patients.
   278. A method of treating B-ALL, comprising administering to a subject in need thereof a therapeutically effective amount of the modified αβT cell of any one of embodiments 210-274 or the pharmaceutical composition of embodiment 275.
   279. The modified αβT cell of any one of embodiments 210-274 or the pharmaceutical composition of embodiment 275 for use in therapy.
   280. The modified αβT cell of any one of embodiments 210-274 or the pharmaceutical composition of embodiment 275 for use in treatment of B-ALL.
   281. Use of the modified cell of any one of embodiments 210-274 or the pharmaceutical composition of embodiment 275 in the manufacturing of a medicament for treatment of B-ALL.
   282. A method of killing cancer cells which are KRAS G12V positive, comprising administering to a subject in need thereof a therapeutically effective amount of the modified αβT cell of any one of embodiments 210-274 or the pharmaceutical composition of embodiment 275.
   283. A method of treating colorectal cancer with KRAS G12V, comprising administering to a subject in need thereof a therapeutically effective amount of the modified αβT cell of any one of embodiments 210-274 or the pharmaceutical composition of embodiment 275.
   284. The modified αβT cell of any one of embodiments 210-274 or the pharmaceutical composition of embodiment 275 for use in therapy.
   285. The modified αβT cell of any one of embodiments 210-274 or the pharmaceutical composition of embodiment 275 for use in treatment of colorectal cancer with KRAS G12V.
   286. Use of the modified cell of any one of embodiments 210-274 or the pharmaceutical composition of embodiment 275 in the manufacturing of a medicament for treatment of colorectal cancer with KRAS G12V.
   287. A method of generating the modified αβT cell of any one of embodiments 210-274, comprising (i) generating the modified iPSC cell of any one of embodiments 210-274, optionally, according to the method of any one of embodiments 72-131, and (ii) differentiating the modified iPSC cell into the modified αβT cell.
   288. A method of generating the modified αβT cell of any one of embodiments 1-69, comprising (i) generating the modified iPSC cell of any one of embodiments 1-69, optionally, according to the method of any one of embodiments 72-131, and (ii) differentiating the modified iPSC cell into the modified αβT cell.
   289. The method of any one of embodiments 72-131 and embodiments 139-145, further comprising differentiating the modified iPSC generated from the method of any one of embodiments 72-131 and embodiments 139-145, into the modified αβT cell.

Also, the present disclosure encompasses, inter alia, the following embodiments, among others disclosed herein.

Embodiment 301: A modified iPSC, αβT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell.

Embodiment 302: The modified cell of embodiment 301, wherein the genomic disruption is an inactivating nucleic acid mutation in the exon 2C, optionally wherein the mutation is an insertion, deletion, or substitution.

Embodiment 303: The modified cell of embodiment 301 or embodiment 302, wherein the genomic disruption is within a target sequence in BIM that comprises any one of SEQ ID NOs: 14, 20, 21, and 22.

Embodiment 304: The modified cell of any one of embodiments 301-303, wherein the genomic disruption within BIM is made by administering a guide RNA to said cell, and optionally wherein an endonuclease or a nucleic acid encoding the endonuclease is also administered to said cell.

Embodiment 305: The modified cell of embodiment 304, wherein the guide RNA comprises any one of SEQ ID NOs: 26, 34, 35 and 36, or a guide sequence differing by no more than 3 nucleotides from any one of SEQ ID Nos: 2, 8, 9 and 10.

Embodiment 306: The modified cell of embodiment 304 or embodiment 305, wherein the guide RNA comprises SEQ ID NO: 36 or a guide sequence differing by no more than 3 nucleotides from SEQ ID NO: 10, and the endonuclease is a MAD7 endonuclease.

Embodiment 307: The modified cell of embodiment 304 or embodiment 305, wherein the guide RNA comprises any one of SEQ ID NOs: 26, 34, and 35, or a guide sequence differing by no more than 3 nucleotides from any one of SEQ ID Nos: 2, 8, and 9, and the endonuclease is a Cas9 endonuclease.

Embodiment 308: The modified cell of any one of embodiments 301-307, comprising a genomic disruption in an endogenous suppressor of cytokine signaling 1 (SOCS1) gene that suppresses or eliminates expression of a functional SOCS1 protein and/or a genomic disruption in an endogenous cytokine-inducible SH2-containing protein (CISH) gene that suppresses or eliminates expression of a functional CISH protein.

Embodiment 309: The modified cell of embodiment 308, wherein the genomic disruption is in exon 2 of the SOCS1 gene; optionally wherein the genomic disruption is an inactivating nucleic acid mutation, optionally wherein the mutation is an insertion, deletion, or substitution.

Embodiment 310: The modified cell of embodiment 308 or embodiment 309, wherein the genomic disruption is in exon 3 or exon 4 of the CISH gene; optionally wherein the genomic disruption is an inactivating nucleic acid mutation, optionally wherein the mutation is an insertion, deletion, or substitution.

Embodiment 311: The modified cell of any one of embodiments 308-310, wherein the genomic disruption is within a target sequence in SOCS1 that comprises SEQ ID NO: 17 or SEQ ID NO: 23 and/or the genomic disruption is within a target sequence in CISH that comprises SEQ ID NO: 15, SEQ ID NO: 16 or SEQ ID NO: 24.

Embodiment 312: The modified cell of any one of embodiments 308-311, wherein the genomic disruption within SOCS1 and CISH is made by administering guide RNAs to said cell, optionally wherein the guide RNAs are single-guide RNAs (sgRNAs) or crRNAs, and wherein an endonuclease or a nucleic acid encoding the endonuclease is also administered to said cell.

Embodiment 313: The modified cell of embodiment 312, wherein the guide RNAs comprise the guide sequence of any one or more of SEQ ID NOs: 5, 11, 3, 4, and 12, or comprises the sgRNA or crRNA sequence of any one or more of SEQ ID NOs: 29, 37, 27, 28, and 38.

Embodiment 314: The modified cell of any one of embodiments 301-313, further comprising a genomic disruption in an endogenous FAS cell surface death receptor (FAS) gene that suppresses or eliminates expression of a functional FAS protein.

Embodiment 315: The modified cell of embodiment 314, wherein the genomic disruption in the FAS gene is in exon 1 of the FAS gene.

Embodiment 316: The modified cell of embodiment 314 or embodiment 315, wherein the genomic disruption is an inactivating nucleic acid mutation in exon 1 of an endogenous FAS gene, optionally wherein the mutation is an insertion, deletion, or substitution.

Embodiment 317: The modified cell of any one of embodiments 314-316, wherein the genomic disruption is within a target sequence in FAS that comprises SEQ ID NO: 62.

Embodiment 318: The modified cell of any one of embodiments 314-317, wherein the genomic disruption within FAS is made by administering a guide RNA to said cell, and optionally wherein an endonuclease or a nucleic acid encoding the endonuclease is also administered to said cell.

Embodiment 319: The modified cell of embodiment 318, wherein the guide RNA comprises the guide sequence of SEQ ID NO: 61, or comprises the sgRNA sequence of SEQ ID NO: 60.

Embodiment 320: The modified cell of any one of embodiments 301-319, further comprising a genomic disruption in an endogenous β-2-Microglobulin (B2M) gene that suppresses or eliminates expression of a functional B2M protein.

Embodiment 321: The modified cell of embodiment 320, wherein the genomic disruption in the B2M gene is in exon 2 of the B2M gene.

Embodiment 322: The modified cell of embodiment 320 or embodiment 321, wherein the genomic disruption is an inactivating nucleic acid mutation in exon 2 of an endogenous B2M gene, optionally wherein the mutation is an insertion, deletion, or substitution.

Embodiment 323: The modified cell of any one of embodiments 320-322, wherein the genomic disruption is within a target sequence in B2M that comprises SEQ ID NO: 56.

Embodiment 324: The modified cell of any one of embodiments 320-323, wherein the genomic disruption within B2M is made by administering a guide RNA to said cell, and optionally wherein an endonuclease or a nucleic acid encoding the endonuclease is also administered to said cell.

Embodiment 325: The modified cell of embodiment 324, wherein the guide RNA comprises the guide sequence of SEQ ID NO: 55, or comprises the sgRNA sequence of SEQ ID NO: 54.

Embodiment 326: The modified cell of any one of embodiments 301-325, further comprising a genomic disruption in an endogenous Class II transactivator (CIITA) gene that suppresses or eliminates expression of a functional CIITA protein.

Embodiment 327: The modified cell of embodiment 326, wherein the genomic disruption in the CIITA gene is in exon 3 of the CIITA gene.

Embodiment 328: The modified cell of embodiment 326 or embodiment 327, wherein the genomic disruption is an inactivating nucleic acid mutation in exon 3 of an endogenous CIITA gene, optionally wherein the mutation is an insertion, deletion, or substitution.

Embodiment 329: The modified cell of any one of embodiments 326-328, wherein the genomic disruption is within a target sequence in CIITA that comprises SEQ ID NO: 59.

Embodiment 330: The modified cell of any one of embodiments 326-329, wherein the genomic disruption within CIITA is made by administering a guide RNA to said cell, and optionally wherein an endonuclease or a nucleic acid encoding the endonuclease is also administered to said cell.

Embodiment 331: The modified cell of embodiment 330, wherein the guide RNA comprises the guide sequence of SEQ ID NO: 58, or comprises the sgRNA sequence of SEQ ID NO: 57.

Embodiment 332: The modified cell of any one of embodiments 301-331, further comprising a genomic disruption in an endogenous T-cell receptor alpha constant region (TRAC) gene that suppresses or eliminates expression of a functional TRAC protein and/or a genomic disruption in an endogenous TRBC1/2 gene that suppresses or eliminates expression of a functional TRBC1/2 protein.

Embodiment 333: The modified cell of any one of embodiments 301-332, wherein the genomic disruption is an inactivating nucleic acid mutation, optionally wherein the mutation is an insertion, deletion, or substitution.

Embodiment 334: The modified cell of any one of embodiments 301-333, further comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).

Embodiment 335: The modified cell of embodiment 334, wherein the amino acid sequence of ECD of the first fusion protein subunit is SEQ ID NO: 63, and the amino acid sequence of ECD of the second fusion protein subunit is SEQ ID NO: 64.

Embodiment 336: The modified cell of embodiment 334 or embodiment 335, wherein the amino acid sequence of intracellular domain of the first fusion protein subunit is SEQ ID NO: 69, and the amino acid sequence of intracellular domain of the second fusion protein subunit is SEQ ID NO: 70.

Embodiment 337: The modified cell of any one of embodiments 334-336, wherein the first fusion protein subunit further comprises a transmembrane domain between the ECD and the intracellular domain thereof, and the second fusion protein subunit further comprises a transmembrane domain between the ECD and the intracellular domain thereof.

Embodiment 338: The modified cell of embodiment 337, wherein the transmembrane domain of the first fusion protein subunit is of the interleukin-2 receptor subunit gamma, and the transmembrane domain of the second fusion protein subunit is of the interleukin-2 receptor subunit beta.

Embodiment 339: The modified cell of embodiment 338, wherein the amino acid sequence of transmembrane domain of the first fusion protein subunit is SEQ ID NO: 67, and the amino acid sequence of transmembrane domain of the second fusion protein subunit is SEQ ID NO: 68.

Embodiment 340: The modified cell of any one of embodiments 334-339, wherein the first fusion protein subunit further comprises a membrane-proximal region between the ECD and the intracellular domain thereof, and the second fusion protein subunit further comprises a membrane-proximal region between the ECD and the intracellular domain thereof.

Embodiment 341: The modified cell of embodiment 340, wherein the membrane-proximal region of the first fusion protein subunit is of the interleukin-2 receptor subunit gamma, and the membrane-proximal region of the second fusion protein subunit is of the interleukin-2 receptor subunit beta.

Embodiment 342: The modified cell of embodiment 341, wherein the amino acid sequence of membrane-proximal region of the first fusion protein subunit is SEQ ID NO: 65, and the amino acid sequence of membrane-proximal region of the second fusion protein subunit is SEQ ID NO: 66.

Embodiment 343: The modified cell of any one of embodiment 334-342, wherein a first exogenous polynucleotide encoding the first fusion protein subunit of the signal converter is inserted into a SOCS1 locus.

Embodiment 344: The modified cell of any one of embodiments 334-343, wherein a second exogenous polynucleotide encoding the second fusion protein subunit of the signal converter is inserted into a ROSA26 locus.

Embodiment 345: The modified cell of any one of embodiments 334-344, the first fusion protein subunit of the signal converter comprises an amino acid sequence of SEQ ID NO: 71, and the second fusion protein subunit of the signal converter comprises an amino acid sequence of SEQ ID NO: 72; optionally the first exogenous polynucleotide encodes an amino acid sequence comprising SEQ ID NO: 75, and the second exogenous polynucleotide encodes an amino acid sequence comprising SEQ ID NO: 76.

Embodiment 346: The modified cell of any one of embodiments 334-345, wherein the amino acid sequence of the first fusion protein subunit is SEQ ID NO: 71, and the amino acid sequence of the second fusion protein subunit is SEQ ID NO: 72.

Embodiment 347: The modified cell of any one of embodiments 301-346, further comprising an exogenous polynucleotide encoding a B2M-HLA-E fusion protein (B2M-HLA-E) comprising at least a portion of B2M fused with at least of a portion of HLA-E.

Embodiment 348: The modified cell of embodiment 347, wherein the exogenous polynucleotide encoding the B2M-HLA-E is inserted into a B2M locus.

Embodiment 349: The modified cell of embodiment 347 or embodiment 348, wherein the B2M-HLA-E comprises an amino acid sequence of SEQ ID NO: 81.

Embodiment 350: The modified cell of any one of embodiments 301-349, further comprising one or more exogenous polynucleotides encoding TCRαβ.

Embodiment 351: The modified cell of embodiment 350, wherein the one or more exogenous polynucleotides encoding the TCRαβ are inserted into a TRAC locus.

Embodiment 352: The modified cell of embodiment 350 or embodiment 351, wherein the one or more exogenous polynucleotides encode the TCRαβ which specifically binds KRAS G12V.

Embodiment 353: The modified cell of embodiment 352, wherein the TCRαβ which specifically binds KRAS G12V comprises an α chain having the amino acid sequence of SEQ ID NO: 79 and a β chain having the amino acid sequence of SEQ ID NO: 80; or the TCRαβ which specifically binds KRAS G12V comprises an α chain having the amino acid sequence of SEQ ID NO: 82 and a β chain having the amino acid sequence of SEQ ID NO: 83.

Embodiment 354: The modified cell of any one of embodiment 301-353, further comprising an exogenous polynucleotide encoding a chimeric antigen receptor (CAR) which specifically binds human CD19 or further comprising an exogenous polynucleotide encoding a chimeric antigen receptor (CAR) which specifically binds human CD20.

Embodiment 355: The modified cell of any one of embodiments 301-353, further comprising an exogenous polynucleotide encoding a chimeric antigen receptor (CAR) which specifically binds human CD19, and further comprising an exogenous polynucleotide encoding a chimeric antigen receptor (CAR) which specifically binds human CD20.

Embodiment 356: The modified cell of embodiment 354 or embodiment 355, wherein the exogenous polynucleotide encoding the CAR which specifically binds human CD19 is inserted into an AAVS1 locus.

Embodiment 356: The modified cell of any one of embodiments 354-356, wherein the CAR which specifically binds human CD19 comprises the amino acid sequence of SEQ ID NO: 93 or SEQ ID NO: 94.

Embodiment 357: The modified cell of any one of embodiments 301-356, further comprising an exogenous polynucleotide encoding a suicide switch, which comprises a herpes simplex virus thymidine kinase (HSV-tk) polypeptide or a functional variant thereof capable of inducing cell death upon administration of Ganciclovir (GCV).

Embodiment 358: The modified cell of embodiments 357, wherein the suicide switch comprises an amino acid sequence of SEQ ID NO: 85.

Embodiment 359: The modified cell of embodiments 357 or embodiment 358, wherein the suicide switch is inserted into a Rogi2 locus, or wherein the suicide switch is inserted into a FAS locus.

### BRIEF DESCRIPTION OF THE DRAWINGS

FIG. 1 shows the knockout efficiency of candidate genes (CISH, SOCS1, BIM and FAS) in KRAS G12V TCR T-iαβT (iPSC-derived αβT) cells examined by Western blotting.

FIG. 2 shows the better effects of CISH^{-/-}SOCS1^{-/-} double knockout (DKO) and BIM^{-/-} CISH^{-/-}SOCS1^{-/-} triple knockout (TKO) KRAS G12V TCR T-iαβT cells in enhanced cytotoxicity and/or persistence against HLA-A11-SW620 cells compared with single KO. FIG. 2 shows the serial killing ability of CISH/single knockout (SKO), SOCS1^{-/-}SKO, CISH^{-/-}SOCS1^{-/-} DKO, and BIM^{-/-}CISH^{-/-}SOCS1^{-/-}TKO KRAS G12V TCR T-iαβT against HLA-A11-SW620 cells at a 4:1 effector:target (E:T) ratio in the presence of IL-2 (0.1 ng/mL) monitored by the Incucyte® SX5 live-cell analysis system.

FIG. 3 shows the enhanced IFN-g secretion in CISH^{-/-}SOCS1^{-/-} double knockout (DKO) and BIM^{-/-}CISH^{-/-}SOCS1^{-/-} triple knockout (TKO) KRAS G12V TCR T-iαβT cells post the 1^{st} round of serial killing against HLA-A11 SW620 cells compared with control and single gene knockout KRAS G12V TCR T-iαβT cells. The concentration of IFN-g in the culture supernatants post co-culturing with SW620 cells was quantified using the Lumit® IFN-g (Human) Immunoassay Kit.

FIG. 4 shows prolonged survival of KRAS G12V TCR T-iαβT cells with a BIM_{EL} and BIM_{L} deficiency (knockout by BIM sgRNA #5 (SEQ ID NO: 26)) or of BIM^{-/-}CISH^{-/-}SOCS1^{-/-} TKO KRAS G12V TCR T-iαβT cells, in which the BIM-modification has a BIM_{EL} and BIM_{L} deficiency, post IL-2 deprivation for 2-8 days, in which the experimental IL-2 levels were 0 ng/mL and 0.1 ng/mL.

FIGS. 5A-5C show that the BIM short splice variant (BIM_{S}) is necessary for T-iαβT cell survival post IL-15 deprivation. FIG. 5A is the schematic representation of BIM gene structure, and there are three main splice variants expressed in T-iαβT cells. FIG. 5B shows a knockout of BIM splice variants with different BIM sgRNAs (BIM #5 (SEQ ID NO: 26), #9 (SEQ ID NO: 32), #19 (SEQ ID NO: 33) or #25 (SEQ ID NO: 35)), as demonstrated by Western blotting. FIG. 5C shows knockout of BIM_{EL} and BIM_{L} splice variants with BIM sgRNA #5 (SEQ ID NO: 26) and #25 (SEQ ID NO: 35) (retaining BIM_{S}) exhibited prolonged survival when T-iαβT cells were cultured at 0, 0.05 and 0.3 ng/mL IL-15 concentration condition.

FIGS. 6A-6C show the editing efficiency of CISH crRNA (CRISPR RNA) using CRISPR/MAD7-mediated gene editing. FIG. 6A shows the insertion-deletion (indel) percentages (ICEd) of the control and CISH crRNA samples calculated by ICE analysis. Data presented as mean±SD. FIG. 6B shows a representative screenshot of the Sanger sequencing chromatogram demonstrating the indel formation after CRISPR/MAD7-mediated gene editing at the CISH locus. The shaded area indicates the targeting site after indel formation by CISH crRNA. FIG. 6C shows the summary chart of single clone selection and analysis for CISH edited cells.

FIGS. 7A-7D show the editing efficiency of SOCS1 crRNA using CRISPR/MAD7-mediated gene editing. FIG. 7A shows the indel percentages (ICEd) of the control and SOCS1 crRNA samples calculated by ICE analysis. Data are presented as mean±SD. FIG. 7C shows a representative screenshot of the Sanger sequencing result demonstrating the indel formation after CRISPR/MAD7-mediated gene editing at the SOCS1 locus. The shaded area indicates the targeting site after indel formation by SOCS1 crRNA. FIG. 7D shows the summary chart of single clone selection and analysis for SOCS1 edited cells. FIG. 7B shows the western blot analysis of SOCS1 protein depletion in two SOCS1 homozygous knockout clones (#13 and #36).

FIGS. 8A-8D show the editing efficiency of BIM crRNA using CRISPR/MAD7-mediated gene editing. FIG. 8A shows the indel percentages (ICEd) of the control and BIM crRNA samples calculated by ICE analysis. Data are presented as mean±SD. FIG. 8C shows a representative screenshot of the Sanger sequencing result demonstrating the indel formation after CRISPR/MAD7-mediated gene editing at the BIM locus. The shaded area indicates the targeting site after indel formation by BIM crRNA. FIG. 8D shows the summary chart of single clone selection and analysis for BIM edited cells. FIG. 8B shows the western blot analysis of BIM protein depletion in one heterozygous (#21) and one homozygous (#39) knockout clone. The BIM designed crRNA was intended to deplete BIM_{EL} and BIM_{L} splice forms but to retain BIM_{S}.

FIG. 9 shows the prolonged survival of FAS-deficient (knockout of the FAS gene by FAS sgRNA #1) KRAS G12V TCR T-iαβT cells in the presence of the FAS ligand (FASL; an inducer of cell apoptosis) and human recombinant IL-2 (0.1 ng/mL), compared with control (nucleofection by Scramble sgRNA #2) KRAS G12V TCR T-iαβT cells.

FIG. 10 shows the enhanced durable cytotoxicity of FAS-deficient KRAS G12V TCR T-iαβT cells against FASL-overexpressed and FAS-deficient HLA-A11 SW620 cells (GFP-HLA-A11-SW620-FASL) at a 4:1 E:T ratio in the presence of IL-2 (1 ng/mL) monitored by the Incucyte® SX5 live-cell analysis system.

FIG. 11 shows the prolonged survival of FAS-deficient TKO (BIM^{-/-}CISH^{-/-}SOCS1^{-/-}) KRAS G12V TCR T-iαβT cells in the presence of the FAS ligand and human recombinant IL-2 (0.1 ng/mL), compared with BIM^{-/-}CISH^{-/-}SOCS1^{-/-} TKO KRAS G12V TCR T-iαβT cells.

FIG. 12 shows the enhanced durable cytotoxicity of FAS-deficient TKO (BIM^{-/-}CISH^{-/-}SOCS1−/−) KRAS G12V TCR T-iαβT cells against FASL-overexpressed and FAS-deficient HLA-A11 SW620 cells at a 4:1 E:T ratio in the presence of IL-2 (0.1 ng/mL), compared with BIM^{-/-}CISH^{-/-}SOCS1^{-/-} TKO KRAS G12V TCR T-iαβT cells monitored by the Incucyte® SX5 live-cell analysis system.

FIG. 13 shows the increased IFN-g secretion in FAS-deficient TKO (BIM^{-/-}CISH^{-/-}SOCS1^{-/-}) KRAS G12V TCR T-iαβT cells post the 1^{st} round of serial killing against FASL-overexpressed and FAS-deficient HLA-A11 SW620 cells compared with BIM^{-/-}CISH^{-/-}SOCS1^{-/-} TKO KRAS G12V TCR T-iαβT cells. The concentration of IFN-g in the culture supernatants post co-culturing with SW620 cells was quantified using the Lumit® IFN-g (Human) Immunoassay Kit.

FIG. 14 illustrates the extracellular domains (ECD) of an interferon gamma (IFN-γ) receptor fused to the intracellular domains (ICD) of a distinct cytokine receptor, with a transmembrane domain positioned in between.

FIGS. 15A-15B illustrate that the IFNγR-IL2/IL15R signal converter is capable of inducing phosphorylation of STAT1 and STAT5 in mature iαβT 4KO (CISH^{-/-}/SOCS1^{-/-}/BIM^{-/-}/FAS^{-/-}) KRAS G12V TCR cells. FIG. 15A shows that the percentage of cells with IFNγR-IL2/IL15R signal converter in mature iαβT 4KO KRAS G12V TCR cells was approximately 35.9%. FIG. 15B shows that, upon IFN-γ stimulation, phosphorylation of STAT1 and STAT5 was markedly increased in mature iαβT 4KO KRAS G12V TCR cells with IFNγR-IL2/IL15R signal converter.

FIG. 16 illustrates enhanced expansion of mature iαβT 4KO (CISH^{-/-}/SOCS1^{-/-}/BIM^{-/-}/FAS^{-/-}) KRAS G12V TCR cells expressing the IFNγR-IL2/IL15R signal converter under repeated tumor antigen stimulation.

FIG. 17 demonstrates the enhanced serial tumor cell killing capacity of mature iαβT 4KO (CISH^{-/-}/SOCS1^{-/-}/BIM^{-/-}/FAS^{-/-}) KRAS G12V TCR cells expressing the IFNγR-IL2/IL15R signal converter against GFP-HLA-A11-SW620 tumor cells.

FIG. 18A-18B demonstrate the decrease in cell viability by GCV treatment of dellta-HSV-tk-A168H knock-in iPSC (FIG. 18A) and iPSC-derived iabT cells (FIG. 18B).

FIG. 19A shows that the percentage of cells expressing IFNγR-IL2/IL15R signal converter was approximately 19.1%. FIG. 19B shows that the percentage of cells expressing CD19 CAR was approximately 98%.

FIG. 20 illustrates cell expansion of 4KO iαβT cells expressing CD19 CAR and IFNγR-IL2/IL15R under repeated Nalm-6-iRFP713 tumor cell stimulation.

FIG. 21 demonstrates the single run tumor cell killing capacity of mature CD19 CAR 4KO (CISH^{-/-}/SOCS1^{-/-}/BIM^{-/-}/FAS^{-/-}) KRAS G12V TCR iαβT cells expressing the IFNγR-IL2/IL15R signal converter against Nalm-6-iRFP713 tumor cells at indicated E:T ratios.

FIG. 22A demonstrates the superior persistent killing of 4KO iαβT cells expressing CD19 CAR and IFNγR-IL2/IL15R against Nalm-6-iRFP713 tumor cells at a 4:1 E:T ratio, compared with that of primary αβT expressing CD19 CAR. FIG. 22B shows the cytokine secretion profile of 4KO iαβT cells expressing CD19 CAR and IFNγR-IL2/IL15R or primary αβT expressing CD19 CAR against Nalm-6-iRFP713 tumor cells.

### DETAILED DESCRIPTION

### I. Definitions

Unless stated otherwise, the following terms and phrases have the meanings described below. The definitions are not meant to be limiting in nature and serve to provide a clearer understanding of certain aspects of the present disclosure.

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

Induced pluripotent stem cell (iPSC): As used herein, the terms “induced pluripotent stem cell” or “iPSC” refer to an induced stem cell that is generated from a somatic cell (e.g., a fibroblast or a T cell) using induced pluripotent reprogramming factors, e.g., Yamanaka factors: Oct3/4, Sox2, Klf4 and c-Myc (see Takahashi K, Yamanaka S. Induction of pluripotent stem cells from mouse embryonic and adult fibroblast cultures by defined factors. Cell. 2006 Aug. 25;126(4):663-76. Further, see Takahashi K, Tanabe K, Ohnuki M, Narita M, Ichisaka T, Tomoda K, Yamanaka S. Induction of pluripotent stem cells from adult human fibroblasts by defined factors. Cell. 2007 Nov. 30;131(5):861-72).

T-iPSC: As used herein, the terms “T-iPSC”, “T cell-derived iPSC”, “T derived iPSC”, “iPSC derived from T,” “iPSC derived from a T cell” and the like refer to an iPSC that is generated from a T cell using induced pluripotent reprogramming factors, wherein the T cell could be a primary T cell or a αβT cell.

αβT-iPSC: As used herein, the terms “αβT-iPSC,” “αβT derived iPSC”, “iPSC derived from αβT” and the like refer to an iPSC that is generated from a αβ (alpha/beta) T cell (αβT cell) using induced pluripotent reprogramming factors.

αβT cell: As used herein, a “αβT cell,” “αβT,” “alpha/betaT,” and the like refers to a type of T cell that has a T-cell receptor (TCR) composed of a α glycoprotein chain and a β glycoprotein chain on the surface of the cell, regardless of how the αβT cell is generated/obtained. The αβT cell can be a primary αβT cell or an αβT cell differentiated from iPSC (iαβT).

iαβT: The terms “iPSC-derived αβT cell,” “iPSC-αβT,” “iαβT,” and the like, as used herein, refers to an αβT cell differentiated from an iPSC.

T-iαβT: The terms “T-iαβT,” “T-iabT,” “T derived iαβT”, and the like, as used herein, refers to a αβT cell differentiated from an iPSC derived from a T cell (T-iPSC).

αβT-iαβT: The terms “αβT-iαβT”, “αβT-iPSC derived αβT” and the like, as used herein, refers to an αβT differentiated from an iPSC derived from an αβ T cell (αβT-iPSC).

“An intermediate cell differentiated from an iPSC” and the like, as used herein, refers to a cell differentiated from an iPSC that possesses the ability to be differentiated into an αβT cell. The intermediate cell differentiated from the iPSC includes but is not limited to an αβT-iHSC, an αβT-iCLP, and/or an immature T-iαβT cell.

iPSC-derived Hematopoietic stem cell (iHSC): As used herein, the terms “iPSC-derived hematopoietic stem cell”, or “iHSC” refer to a hematopoietic stem cell generated from an iPSC.

αβT-iPSC Hematopoietic stem cell (αβT-iHSC): As used herein, the terms “αβT-iPSC hematopoietic stem cell” or “αβT-iHSC” refer to a hematopoietic stem cell generated from a αβT-iPSC.

iPSC-derived Common lymphoid progenitor (iCLP cell): As used herein, the terms “iPSC-derived common lymphoid progenitor,” “iCLP cell,” or “iCLP” refer to a common lymphoid progenitor cell that is differentiated from an iHSC, in which the iHSC is generated from an iPSC.

αβT-iPSC Common lymphoid precursor (αβT-iCLP): As used herein, the terms “αβT-iPSC common lymphoid precursor,” “αβT-iCLP cell,” or “αβT-iCLP” refer to a common lymphoid precursor cell that is differentiated from a T-iHSC, in which the T-iHSC is generated from a αβT-iPSC.

iPSC-derived immature T cell (immature iT cell): As used herein, the terms “iPSC-derived immature T cell” or “immature iT cell” refer to an immature T cell differentiated from an iCLP cell, in which the iCLP cell is differentiated from an iHSC, in which the iHSC is generated from an iPSC.

αβ-TiPSC immature alpha/beta T cell (immature T-iαβT cell): As used herein, the terms “αβT-iPSC immature alpha/beta T cell” or “immature T-iαβT cell” refer to an immature αβT cell differentiated from a T-iCLP, in which the T-iCLP is differentiated from a T-iHSC, in which the T-iHSC is generated from a αβT-iPSC.

Genomic Disruption: As used herein, the term “genomic disruption” refers to any genetic alteration (e.g., substitution, deletion, and/or insertion) in the sequence of a gene or epigenetic modification (e.g., DNA methylation, histone modification) without changes to the sequence of the gene. The genomic disruption can result in a change of the transcription of a gene, translation of mRNA, and/or function of a protein. A genomic disruption can include alterations to a coding sequence, a non-coding sequence (e.g., promoter, enhancer, insulator, operon, or silencer), or a combination thereof.

Splice Variant: As used herein, the term “splice variant” refers to mature mRNA and its encoded protein produced by alternatively splicing a gene, e.g., the BIM gene, in which exons of pre-mRNA are reconnected in at least two distinct forms during the process of RNA splicing. The BIM gene is transcribed as three splice variants: BIM_{EL}, which contains coding exons E1, E2A, E2B, E2C, E4 and E5; BIM_{L}, which contains coding exons E1, E2A, E2C, E4 and E5; and BIM_{S}, which contains coding exons E1, E2A, E4 and E5.

Identity: As used herein, the term “identity” in the context of sequence comparisons refers to the number of exact matches between two different sequences in a sequence alignment. Sequence alignment techniques and software include Basic Local Alignment Search Tool (BLAST, which includes e.g., BLASTP for protein sequences and BLASTN for nucleic acid sequences), ClustalOmega, MUSCLE, and MAFFT.

Bcl-2 Interacting Mediator of cell death (BIM): As used herein, the terms “Bcl-2 Interacting Mediator of cell death,” “BIM,” “Human BIM gene,” “Gene ID: 10018,” or “BCL2L11” refer to a member of the BH3 (Bcl-2 homology 3)-only proteins, which can directly activate the proapoptotic effector proteins BAX and BAK. BIM can also indirectly activate BAX and BAK by binding to antiapoptotic BCL-2 family members (BCL-2, BCL-xL, MCL-1 and A1). BIM is transcribed as three splice variants, BIM_{EL} (NM_138621.5; NP_619527.1, containing coding exons E1, E2A, E2B, E2C, E4 and E5), BIM_{L} (NM_006538.5; NP_006529.1, containing coding exons E1, E2A, E2C, E4 and E5) and BIM_{S} (NM_001204106.1; NP_001191035.1, containing coding exons E1, E2A, E4 and E5).

Cytokine-inducible SH2-containing protein (CISH): As used herein, the terms “CISH,” “Cytokine-inducible SH2-containing protein,” “CIS1,” “Gene ID: 1154,” “NM_013324.7,” or “NP_037456.5” refer to a member of the suppressor of cytokine signaling (SOCS) protein family. The SOCS family members are negative regulators of cytokine signal transduction that inhibit the JAK/STAT pathway. Each SOCS family member contains a central SH2 domain and a conserved carboxy-terminal motif designated as the SOCS box. These proteins are important regulators of cytokine signaling, proliferation, differentiation, and immune responses.

Suppressor of cytokine signaling 1 (SOCS1): As used herein, the terms “Suppressor of cytokine signaling 1,” “SOCS1,” “Gene ID: 8651,” “NM_003745.2,” “NP_003736.1” “Janus kinase binding protein,” “JAB,” “Stat-induced Stat inhibitor-1,” “SSI-1,” “Tec-interacting protein 3,” or “TIP3” refer to a cytokine-regulated SOCS family member that directly inhibits JAK family members through interaction within its kinase activation loop. In addition to inhibiting JAK/STAT signaling, SOCS1 can also negatively regulate Toll-like receptors that contribute to innate immunity.

FAS cell surface death receptor: As used herein, the terms “FAS cell surface death receptor,” “FAS,” “Gene ID: 355,” “NM_000043.6,” and “NP_000034.1” refer to a tumor necrosis factor (TNF) superfamily death receptor that induce caspase-dependent apoptosis following engagement with its extracellular ligand (FAS ligand (FASL)).

β-2-Microglobulin: As used herein, the terms “β-2-Microglobulin,” “Beta-2-Microglobulin,” “B2M,” “Gene ID: 567,” “NM_004048,” and “NP_004039” refer to a component of the human leukocyte antigen (HLA) class I molecules found on the surface of nearly all nucleated cells. In addition, β2M associates non-covalently with the HLA class I heavy chain, enabling proper folding and stabilization of HLA class I.

Class II transactivator: As used herein, the terms “Class II transactivator,” “CIITA,” “Gene ID: 4261,” “NM_001286402.1,” and “NP_001273331.1” refer to a master regulator of HLA class II expression by recruiting and facilitating the assembly of DNA-binding proteins, including the regulatory factor X (RFX) complex, cAMP response element-binding protein (CREB), and nuclear factor Y (NF-Y).

Clustered Regularly Interspace Short Palindromic Repeats (CRISPR): As used herein, the terms “Clustered Regularly Interspace Short Palindromic Repeats” or “CRISPR” refer to a gene editing system that utilizes a guide sequence and a nuclease to modify a cell's genome at a specific location. The nuclease cuts the cell's genome at the specific location targeted by the guide sequence, allowing a target sequence to be removed, a sequence to be added, or a combination thereof.

Programmable Addition Via Site-Specific Target Elements (PASTE): As used herein, the terms “Programmable Addition Via Site-Specific Target Elements” or “PASTE” refer to a gene editing system that utilizes a guide sequence and a CRISPR-Cas9 nickase fused to a reverse transcriptase and a serine integrase to modify a cell's genome at a specific location. The nickase cuts one strand of double stranded DNA of the cell's genome at the specific location targeted by the guide sequence, allowing a target sequence to be removed, a sequence to be added using the serine integrase, or a combination thereof.

Prime Editing: As used herein, the term “Prime editing” refers to a gene editing system that utilizes a guide sequence and a catalytically impaired Cas nickase (e.g., Cas9 nickase or Cas12 nickase) fused to a reverse transcriptase to modify a cell's genome at a specific location. The nickase cuts one strand of double stranded DNA of the cell's genome at the specific location targeted by the guide sequence, allowing a target sequence to be removed, a sequence to be added using the reverse transcriptase, or a combination thereof.

Base Editing: As used herein, the term “Base editing” refers to a gene editing system that utilizes a guide sequence and a base editor, which is a Cas nickase (e.g., Cas9 nickase or Cas12 nickase) and a nucleoside deaminase, including e.g., a cytosine deaminase or an adenine deaminase, to modify a cell's genome at a specific location. The nickase cuts one strand of double stranded DNA of the cell's genome at the specific location targeted by the guide sequence, allowing the nucleoside deaminase to remove an amino group from a specific type of nucleoside (e.g., cytosine or adenine) at a specific location.

Transcription Activator-like Effector Nuclease (TALEN): As used herein, the terms “Transcription Activator-like Effector Nuclease” or “TALEN” refer to a gene editing system that utilizes an endonuclease, including e.g., Fokl, fused to a transcription activator-like effector (TALE) that is engineered to bind to a desired DNA sequence to promote DNA cleavage by the endonuclease at the specific location designated by the TALE.

Zinc-finger nuclease (ZFN): As used herein, the terms “Zinc-finger nuclease” or “ZFN” refer to a gene editing system that utilizes an endonuclease, including e.g., Fokl, fused to a plurality of zinc fingers. Each zinc finger targets a specific nucleotide sequence at which the endonuclease generates a double-stranded DNA break.

Endonuclease: As used herein, the term “endonuclease” refers to an enzyme that cleaves a phosphodiester bond of a polynucleotide molecule. An endonuclease that targets a specific sequence of nucleotides is a restriction endonuclease. As used herein, the terms “homing endonuclease” and “meganuclease” refer to an endonuclease that targets specific asymmetric sequences of about 12 to and 45 base pairs in length, usually about 14 to about 25 base pairs in length.

Transfection: As used herein, the term “transfection” refers to a variety of techniques for introducing a foreign nucleic acid molecule (e.g., DNA) into a host cell, including electroporation, physical transfection (e.g., microinjection, particle bombardment, or phototransfection), lipid-mediated transfection, diethylaminoethyl (DEAE)-dextran transfection, calcium phosphate precipitation, cationic polymer transfection, or viral transfection.

Interferon-gamma receptor: As used herein, the terms “interferon-gamma receptor,” “IFNGR,” and “IFN-γ receptor” refer to a cytokine receptor comprising a heterodimer. The heterodimer of IFNGR comprises interferon-gamma receptor 1 (IFNGR1) and interferon-gamma receptor 2 (IFNGR2). The interferon-gamma cytokine binds to the IFNGR.

CD132: As used herein, the terms “CD132,” “the common γ chain (γc) of interleukin-2 receptor,” and “IL2RG” refer to a cytokine receptor subunit that is common to the cytokine receptor complexes of at least IL-2, IL-15, IL-4, IL-7, IL9, and IL-21. CD132 comprises an intracellular domain, a transmembrane domain, and a membrane-proximal region.

Interleukin-2 receptor beta subunit: As used herein, the terms “interleukin-2 receptor beta subunit,” “interleukin-2 receptor subunit beta,” “CD122,” and “IL2RB” refer to a cytokine receptor subunit of the cytokine receptor complex of IL-2.

As used herein, the terms “IFNγR-IL2/IL15R”, “IFNγR-IL2/IL15R signal converter”, “IFNγR-IL2/IL15R SC”, “IFNγR-IL2R”, “IFNγR-IL2R signal converter” and “IFNγR-IL2R SC” refer to a chimeric signaling converter comprising a heterodimer. It comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).

As used herein, “unmodified control” and “unmodified cell” refer to a control cell relative to which a comparison is performed. In some embodiments, the unmodified control is the same as the modified cell herein, except that the unmodified control expresses endogenous levels of BIM splice variants as that of a wild-type cell (e.g., αβT cell). In some embodiments, the unmodified control is the same as the modified cell herein, except that the unmodified control expresses endogenous level of a specific protein or protein combination (under comparison) as that of a wild-type cell (e.g., αβT cell). The unmodified control can also have further modifications (other than the protein(s) under comparison) as those in the modified cell to be compared with.

As used herein, “suppresses or eliminates” means any reduction in the level of a functional protein expressed from a gene as compared to a non-modified control.

As used herein, “inactivating nucleic acid mutation” means any nucleic acid mutation causing any reduction in the level of a functional protein expressed from a gene as compared to a non-modified control.

B2M-HLA-E: As used herein, the terms “B2M-HLA-E”, “B2M-HLAE”, “B2M-HLA-E fusion protein” refer to a fusion protein comprising at least a portion of B2M and a portion of HLA-E, wherein at least a portion of B2M and a portion of HLA-E is connected directly or indirectly via a linker. In some embodiments, B2M-HLA-E is able to engage the inhibitory receptors on the surface of NK cells. In some embodiments, B2M-HLA-E comprises an amino acid sequence of SEQ ID NO: 81.

TRAC: As used herein, the term “TRAC” refers to the T-cell receptor alpha constant region gene. It is part of the T-cell receptor (TCR) complex, which is responsible for recognizing antigens in the context of the major histocompatibility complex (MHC) on antigen-presenting cells.

TRBC1/2: As used herein, the term “TRBC1/2” or (TRBC1/TRBC2) refers to the T-cell receptor beta constant region 1 gene and/or T-cell receptor beta constant region 2 gene. TRBC1 and TRBC2 are genes that encode the beta constant region of the T-cell receptor (TCR). Each TCR irreversibly selects one of TRBC1 and TRBC2 for expression, in a random and mutually exclusive manner.

“Kirsten rat sarcoma viral oncogene homolog (KRAS)” refers to an important member of the RAS protein family. The upstream of KRAS is regulated by the epidermal growth factor receptor EGFR family. The EGFR signal can activate the SOS protein, thereby regulating the activation of KRAS. The inactivation and activation state transitions of the KRAS protein in the cell are determined by the molecules it binds to. The guanine nucleotide exchange factor GEF catalyzes KARS to bind to GTP and activate KRAS; while the GTPase activating protein GAP can promote the hydrolysis of GTP bound to KRAS into GDP and lead to KRAS inactivation. Activated KRAS regulates its downstream signaling pathways such as MAPK and PI3K that are related to cell proliferation, cell migration and other functions. KRAS mutations cause it to continuously bind to GTP and remain activated, leading to continuous activation of downstream signaling pathways, thereby promoting tumorigenesis.

“KRAS_G12V” refers to a KRAS protein with a G12V mutation, which can be specifically detected by the immune system of an organism. “G12V” or “G12V mutation” are used interchangeably and refer to a KRAS protein in which the glycine at position 12 is replaced by valine.

“T cell receptor (TCR)” is the protein(s) on the surface of T cells that is responsible for the specific recognition of antigenic peptides bound to MHC (major histocompatibility complex). When TCR binds to antigenic peptides and MHC, T lymphocytes are activated through signal transduction and enter the subsequent immune response process. There are 4 TCR genes in the human genome: two encoding light chain TCRs: TRA gene encodes TCRα, TRG gene encodes TCRγ; two encoding heavy chain TCRs: TRB gene encodes TCRβ, TRD gene encodes TCRδ. Heavy chain TCR and light chain TCR form heterodimers to form a complete TCR. There are two types of TCR in humans: TCRα/β and TCRγ/δ, of which 95% of T cells express TCRα/β, called αβT cells; 5% of T cells express TCRγ/δ, called γ/δT cells. This ratio changes during individual development and in diseased states (such as leukemia), and varies between species. Each chain comprises two extracellular domains: a Variable (V) region and a Constant (C) region. The Constant region is proximal to the cell membrane, followed by a transmembrane region and a short cytoplasmic tail, while the Variable region binds to the peptide/MHC complex. The Variable domain of both the TCRα and TCRβ each have three complementarity-determining regions (CDRs), which play a major role in antigen recognition. Among them, CDR1 and CDR2 are relatively conservative and are responsible for recognizing MHC; CDR3 is the main CDR responsible for recognizing antigens.

“TCRαβ” or “TCRα/β” comprises a TCR a chain or a functional fragment thereof and a TCR β chain or a functional fragment thereof.

“Anti-KRAS G12V TCR”, “anti-KRAS G12V TCRαβ”, or “KRAS G12V TCR” refer to TCRαβ which specifically binds KRAS G12V.

The term “CAR” and “chimeric antigen receptor” refers to a chimeric receptor engineered to endow them with antigen specificity while retaining or enhancing their ability to recognize, trigger signaling pathway and kill a target cell. Chimeric antigen receptor may comprise, for example, (i) an extracellular binding domain, comprising an antigen-specific component, (e.g., a scFv binding to a specific antigen) and optionally a hinge region; (ii) a transmembrane domain; and (iii) intracellular signaling domain, e.g., one or more costimulatory domains and one or more activation domains. Each domain may be heterogeneous, i.e., comprised of sequences derived from (or corresponding to) different protein chains.

The term “suicide switch” refers to a genetically encoded, inducible safety mechanism introduced into engineered cells (e.g., CAR-T, TCR-T, or other modified immune cells) that allows the selective elimination of those cells upon administration of a specific activating agent.

The term “HSV-TK” or “Herpes Simplex Virus thymidine kinase” is a viral enzyme that phosphorylates nucleoside analogues such as Ganciclovir (GCV), converting them into toxic triphosphate metabolites that become incorporated into DNA and trigger apoptosis. An HSV-tk polypeptide or a functional variant thereof refers to the wild-type HSV-tk as well as any engineered, truncated, mutated, or otherwise modified version thereof that preserves the functional capacity to phosphorylate GCV or related nucleoside analogues and thereby induce cell death.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, the term “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The term “therapeutically effective amount” as herein used, refers to the amount of a therapeutic agent that, when administered to a subject for treating a disease, or at least one of the clinical symptoms of a disease or disorder, is sufficient to effect such treatment for the disease, disorder, or symptom. The “therapeutically effective amount” can vary with the therapeutic agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

### II. Modified Cells and Incorporated Components

Disclosed herein are modified αβT-cells, e.g., iPSC-derived αβT-cells, and their precursors (e.g., an iPSC (e.g., αβT-iPSC)), or an intermediate cell differentiated from an iPSC (e.g., iHSC, iCLP, and immature iαβT cells)).

Modified Cells with One or More Genomic Disruptions

In one aspect, such modified cells comprise one or more genomic disruptions selected from the following: 1) a genomic disruption in the SOCS1 gene, 2) a genomic disruption in the CISH gene, 3) a genomic disruption in the BIM gene, 4) a genomic disruption in the β2M gene, 5) a genomic disruption in the CIITA gene, 6) a genomic disruption in the FAS gene, and 7) a genomic disruption in the TRAC gene and/or a genomic disruption in the TRBC1/2 gene.

In another aspect, such modified cells (e.g., iPSC or αβT cell) comprise at least two genomic disruptions—1) in the suppressor of cytokine signaling 1 (SOCS1) gene; and 2) in the cytokine-inducible sh2-containing protein (CISH) gene. In some embodiments, the modified cells comprise at least three genomic disruptions—1) in the SOCS1 gene, 2) in the CISH gene, and 3) in the Bcl-2 interacting mediator of cell death (BIM) gene. In some embodiments, the modified cells comprise at least four genomic disruptions—1) in the SOCS1 gene, 2) in the CISH gene, 3) in the BIM gene, and, for example, 4) in the FAS cell surface death receptor (FAS) gene. In some embodiments, the modified cells comprise at least five genomic disruptions—1) in the SOCS1 gene, 2) in the CISH gene, 3) in the BIM gene, and, for example, 4) in the β-2-Microglobulin (B2M) gene, and 5) in the class II transactivator (CIITA) gene. In some embodiments, the modified cells comprise at least six genomic disruptions—1) in the SOCS1 gene, 2) in the CISH gene, 3) in the BIM gene, and, for example, 4) in the B2M gene, 5) in the CIITA gene, and 6) in the FAS gene. In some embodiments, the modified cells comprise at least seven genomic disruptions—1) in the SOCS1 gene, 2) in the CISH gene, 3) in the BIM gene, and, for example, 4) in the β2M gene, 5) in the CIITA gene, 6) in the FAS gene, and 7) in the TRAC gene and/or a genomic disruption in the TRBC1/2 gene.

In another aspect, the modified cell (e.g., iPSC or αβT cell) comprises at least two genomic disruptions—1) a genomic disruption in the SOCS1 gene (e.g., in exon 2 of the SOCS1 gene), and 2) a genomic disruption in the CISH gene (in exon 3 of the CISH gene). In some embodiments, the modified cell (e.g., iPSC or αβT cell) comprises at least three genomic disruptions—1) a genomic disruption in the SOCS1 gene (e.g., in exon 2 of the SOCS1 gene), 2) a genomic disruption in the CISH gene (in exon 3 of the CISH gene), and 3) a genomic disruption in the BIM gene (e.g., in exon 2C of the BIM gene). In some embodiments, the modified cell (e.g., iPSC or αβT cell) comprises at least four genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), and, for example, 4) a genomic disruption in the FAS gene. In some embodiments, the modified cell (e.g., iPSC or αβT cell) comprises at least five genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the β2M gene, and 5) a genomic disruption in the CIITA gene. In some embodiments, the modified cell (e.g., iPSC or αβT cell) comprises at least six genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the β2M gene, and 6) a genomic disruption in the CIITA gene. In some embodiments, the modified cell (e.g., iPSC or αβT cell) further comprises at least seven genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the β2M gene, 6) a genomic disruption in the CIITA gene, and 7) a genomic disruption in the TRAC gene and/or a genomic disruption in the TRBC1/2 gene.

Modified Cells with One or More Genomic Disruptions and One or More Polynucleotide Incorporations

In one aspect, the modified cell (e.g., iPSC or αβT cell) comprises one or more modifications selected from the following: 1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, 6) a genomic disruption in the CIITA gene, 7) a genomic disruption in the TRAC gene and/or a genomic disruption in the TRBC1/2 gene, 8) an exogenous polynucleotide encoding B2M-HLA-E (e.g., inserted into a B2M locus), and 9) one or more exogenous polynucleotides encoding a signal converter comprising a first fusion protein subunit and a second fusion protein subunit (e.g., the first fusion protein subunit of the signal converter is inserted into a SOCS1 locus and the second fusion protein subunit of the signal converter is inserted into a ROSA26 locus, or vice versa), and 10) an exogenous polynucleotide encoding a suicide switch, which comprises a herpes simplex virus thymidine kinase (HSV-tk) polypeptide or a functional variant thereof capable of inducing cell death upon administration of Ganciclovir (GCV).

In another aspects, in addition to the genomic disruption combination described above, the modified cell (e.g., iPSC or αβT cell) further comprises at least one or two of the following: 1) an exogenous polynucleotide encoding B2M-HLA-E (e.g., inserted into a B2M locus), and 2) one or more exogenous polynucleotides encoding a signal converter.

In another aspects, in addition to the genomic disruption combination described above, the modified cell (e.g., iPSC or αβT cell) further comprises 1) an exogenous polynucleotide encoding B2M-HLA-E (e.g., inserted into a B2M locus), and 2) one or more exogenous polynucleotides encoding a signal converter, and optionally 3) an exogenous polynucleotide encoding a suicide switch, which comprises a herpes simplex virus thymidine kinase (HSV-tk) polypeptide or a functional variant thereof capable of inducing cell death upon administration of Ganciclovir (GCV).

In one embodiment, the modified cell (e.g., iPSC or αβT cell) further comprises all the modifications selected from the following: 1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, 6) a genomic disruption in the CIITA gene, 7) a genomic disruption in the TRAC gene and/or a genomic disruption in the TRBC1/2 gene, 8) an exogenous polynucleotide encoding B2M-HLA-E (e.g., inserted into a B2M locus), and 9) one or more exogenous polynucleotides encoding a signal converter comprising a first fusion protein subunit and a second fusion protein subunit (e.g., the first fusion protein subunit of the signal converter is inserted into a SOCS1 locus and the second fusion protein subunit of the signal converter is inserted into a ROSA26 locus, or vice versa), and optionally 10) an exogenous polynucleotide encoding a suicide switch, which comprises a herpes simplex virus thymidine kinase (HSV-tk) polypeptide or a functional variant thereof capable of inducing cell death upon administration of Ganciclovir (GCV).

In some embodiments, the modified cell is a αβT cell. In some embodiments, the αβT cell can be modified before reprogramming to an iPSC. In some embodiments, the modified cell is an iPSC-derived αβT cell (iαβT). In some embodiments, the αβT cell can be a primary αβT cell. In some embodiments, the modified αβT cell is differentiated from the modified iPSC.

In some embodiments, the modified cell is an iPSC. In some embodiments, a somatic cell (e.g., T cell or PBMC) can be modified before reprogramming to an iPSC. An iPSC is a PSC that is generated from a somatic cell using induced pluripotent reprogramming factors. In some embodiments, the somatic cell is a T cell, including a αβ (alpha/beta) T cell, a peripheral blood mononuclear cell (PBMC), a fibroblast, or a cord blood cell.

In some embodiments, the modified cell is an iPSC derived from a primary T cell (T-iPSC).

In some embodiments, the modified cell is a αβT-iPSC. A αβT-iPSC is an iPSC that is generated from a αβ (alpha/beta) T cell (αβT cell) using induced pluripotent reprogramming factors.

In some embodiments, the modified cell is a primary αβT cell. In some embodiments, the modified cell is an iPSC-derived αβT (iαβT) cell. In some embodiments, the modified cell is an iαβT cell from a T-iPSC (T-iαβT). In some embodiments, the modified cell is a αβT cell from αβT-iPSC (αβT-iαβT).

In some embodiments, the modified cell is an iHSC. An iHSC refers to a hematopoietic stem cell generated from an iPSC. In some embodiments, the modified cell is a αβT-iHSC. An αβT-iHSC refers to a hematopoietic stem cell generated from a αβT-iPSC.

In some embodiments, the modified cell is an iCLP cell, which is a common lymphoid progenitor cell that is differentiated from an iHSC. In some embodiments, the modified cell is a αβT-iCLP, which is a common lymphoid precursor cell that is differentiated from a αβT-iHSC.

In some embodiments, the modified cell is an immature iT cell, which is an immature T cell that is differentiated from an iHSC and/or an iCLP cell. In some embodiments, the modified cell is an immature T-iαβT cell, which is an immature αβT cell that is differentiated from a αβT-iCLP.

In some embodiments, one or more of the SOCS1, CISH, BIM, FAS, B2M, CIITA, TRAC gene, TRBC1/2 gene are genomically disrupted in a αβT-iPSC, αβT-iHSC, αβT-iCLP, or immature T-iαβT cell before differentiation into the T-iαβT cell.

In some embodiments, the SOCS1 and CISH genes are genomically disrupted in a αβT-iPSC, αβT-iHSC, αβT-iCLP, or immature T-iαβT cell before differentiation into the T-iαβT cell. In some embodiments, the SOCS1, CISH, and BIM genes are genomically disrupted in a αβT-iPSC, αβT-iHSC, αβT-iCLP, or immature T-iαβT cell before differentiation into the T-iαβT cell. In some embodiments, the genomic disruptions of the SOCS1 and CISH genes each occur in distinct cell differentiation stages, e.g., the SOCS1 gene is genomically disrupted in the T-iCLP cell and the CISH gene is genomically disrupted in the immature T-iαβT cell that is differentiated from the T-iCLP containing the genomically disrupted SOCS1 gene. A cell differentiation stage is any one of the following cell types: a stem cell having self-renewal and pluripotency properties (e.g., an induced pluripotent stem cell), an adult stem cell (e.g., hematopoietic stem cell) that is differentiated from the stem cell having self-renewal and pluripotency properties; a progenitor cell (e.g., a common lymphoid progenitor) that is differentiated from the adult stem cell; a specific type of cell (e.g., an immature T cell) that is differentiated from the progenitor cell; and a mature specific type of cell (e.g., a αβT cell) that is differentiated from the specific type of cell. A cell differentiation stage is any one of αβT-iPSC, T-iHSC, T-iCLP, immature T-iαβT cell, or T-iαβT cell.

In some embodiments, the genomic disruptions of the SOCS1, CISH, and BIM genes each occur in distinct cell differentiation stages, e.g., the SOCS1 gene is genomically disrupted in the T-iHSC, the CISH gene is genomically disrupted in the T-iCLP differentiated from the T-iHSC containing the genomically disrupted SOCS1 gene, and the BIM gene is genomically disrupted in the immature T-iαβT cell that is differentiated from the T-iCLP containing the SOCS1 and CISH genomic disruptions. In some embodiments, genomic disruptions of two of the genes selected from SOCS1, CISH, and BIM occur in a single cell differentiation stage and a third genomic disruption of the remaining undisrupted gene occurs in a second cell differentiation stage, e.g., the SOCS1 and CISH genes are genomically disrupted in the T-iHSC and the BIM gene is genomically disrupted in the T-iCLP differentiated from the T-iHSC containing the genomically disrupted SOCS1 and CISH genes. In some embodiments, genomic disruptions of two of the genes selected from SOCS1, CISH, and BIM occur in a single cell differentiation stage. In some embodiments, genomic disruptions in each of the three genes selected from SOCS1, CISH, and BIM occur in a single cell differentiation stage.

In some embodiments, the cells comprise genomic disruptions of the SOCS1, CISH, BIM, and FAS genes. In some embodiments, the genomic disruptions of the SOCS1, CISH, BIM, and FAS genes each occur in a distinct cell differentiation stage. In some embodiments, genomic disruptions of three of the genes selected from FAS, SOCS1, CISH, and BIM occur in a single cell differentiation stage and a fourth genomic disruption of the remaining undisrupted gene occurs in a second cell differentiation stage. In some embodiments, genomic disruptions of two of the genes selected from FAS, SOCS1, CISH, and BIM occur in a single cell differentiation stage. In some embodiments, a genomic disruption of one of the genes selected from FAS, SOCS1, CISH, and BIM occurs in a single cell differentiation stage. In some embodiments, genomic disruptions in each of the four genes selected from FAS, SOCS1, CISH, and BIM occur in a single cell differentiation stage.

In some embodiments, the cells comprise genomic disruptions of the SOCS1, CISH, BIM, B2M and CIITA genes. In some embodiments, the genomic disruptions of the SOCS1, CISH, BIM, B2M and CIITA genes each occur in a distinct cell differentiation stage. In some embodiments, genomic disruptions of four of the genes selected from SOCS1, CISH, BIM, B2M and CIITA occur in a single cell differentiation stage. In some embodiments, genomic disruptions of three of the genes selected from SOCS1, CISH, BIM, B2M and CIITA occur in a single cell differentiation stage. In some embodiments, genomic disruptions of two of the genes selected from SOCS1, CISH, BIM, B2M and CIITA occur in a single cell differentiation stage. In some embodiments, a genomic disruption of one of the genes selected from SOCS1, CISH, BIM, B2M and CIITA occurs in a single cell differentiation stage. In some embodiments, genomic disruptions in each of the genes selected from SOCS1, CISH, BIM, B2M and CIITA occur in a single cell differentiation stage.

In some embodiments, the cells comprise genomic disruptions of the SOCS1, CISH, BIM, B2M, CIITA, and FAS genes. In some embodiments, the genomic disruptions of the SOCS1, CISH, BIM, B2M, CIITA, and FAS genes each occur in a distinct cell differentiation stage. In some embodiments, genomic disruptions of five of the genes selected from SOCS1, CISH, BIM, B2M, CIITA, and FAS occur in a single cell differentiation stage. In some embodiments, genomic disruptions of four of the genes selected from SOCS1, CISH, BIM, B2M, CIITA, and FAS occur in a single cell differentiation stage. In some embodiments, genomic disruptions of three of the genes selected from SOCS1, CISH, BIM, B2M, CIITA, and FAS occur in a single cell differentiation stage. In some embodiments, genomic disruptions of two of the genes selected from SOCS1, CISH, BIM, B2M, CIITA, and FAS occur in a single cell differentiation stage. In some embodiments, a genomic disruption of one of the genes selected from SOCS1, CISH, BIM, B2M, CIITA, and FAS occur in a single cell differentiation stage. In some embodiments, genomic disruptions in each of the genes selected from SOCS1, CISH, BIM, B2M, CIITA, and FAS occur in a single cell differentiation stage.

In some embodiments, one or more of the SOCS1, CISH, BIM, FAS, B2M, and CIITA genes are genomically disrupted in an iPSC cell. In some embodiments, the SOCS1 and CISH genes are genomically disrupted in the iPSC cell. In some embodiments, the SOCS1, CISH, and BIM genes are genomically disrupted in the iPSC cell. In some embodiments, the FAS, SOCS1, CISH, and BIM genes are genomically disrupted in the iPSC cell. In some embodiments, the β2M, CIITA, SOCS1, CISH, and BIM genes are genomically disrupted in the iPSC cell. In some embodiments, the FAS, B2M, CIITA, SOCS1, CISH, and BIM genes are genomically disrupted in the iPSC cell.

In some embodiments, one or more of the SOCS1, CISH, BIM, FAS, B2M, and CIITA genes are genomically disrupted in a αβT-iPSC cell. In some embodiments, the SOCS1 and CISH genes are genomically disrupted in the αβT-iPSC cell. In some embodiments, the SOCS1, CISH, and BIM genes are genomically disrupted in the αβT-iPSC cell. In some embodiments, the FAS, SOCS1, CISH, and BIM genes are genomically disrupted in the αβT-iPSC cell. In some embodiments, the β2M, CIITA, SOCS1, CISH, and BIM genes are genomically disrupted in the αβT-iPSC cell. In some embodiments, the FAS, B2M, CIITA, SOCS1, CISH, and BIM genes are genomically disrupted in the αβT-iPSC cell.

In some embodiments, one or more of the SOCS1, CISH, BIM, FAS, B2M, and CIITA genes are genomically disrupted in an iPSC-derived αβT cell (iαβT cell). In some embodiments, the SOCS1 and CISH genes are genomically disrupted the iPSC-derived αβT cell (iαβT cell). In some embodiments, the SOCS1, CISH, and BIM genes are genomically disrupted in the iαβT cell. In some embodiments, the FAS, SOCS1, CISH, and BIM genes are genomically disrupted in the iαβT cell. In some embodiments, the β2M, CIITA, SOCS1, CISH, and BIM genes are genomically disrupted in the iαβT cell. In some embodiments, the FAS, B2M, CIITA, SOCS1, CISH, and BIM genes are genomically disrupted in the iαβT cell.

In some embodiments, one or more of the SOCS1, CISH, BIM, FAS, B2M, and CIITA genes are genomically disrupted in a T-iPSC-derived αβT cell (T-iαβT cell). In some embodiments, the SOCS1 and CISH genes are genomically disrupted the T-iPSC-derived αβT cell (T-iαβT cell). In some embodiments, the SOCS1, CISH, and BIM genes are genomically disrupted in the T-iPSC-derived αβT cell (T-iαβT cell). In some embodiments, the FAS, SOCS1, CISH, and BIM genes are genomically disrupted in the T-iPSC-derived αβT cell (T-iαβT cell). In some embodiments, the β2M, CIITA, SOCS1, CISH, and BIM genes are genomically disrupted in the T-iPSC-derived αβT cell (T-iαβT cell). In some embodiments, the FAS, B2M, CIITA, SOCS1, CISH, and BIM genes are genomically disrupted in the T-iPSC-derived αβT cell (T-iαβT cell).

In some embodiments, one or more of the SOCS1, CISH, BIM, FAS, B2M, and CIITA genes are genomically disrupted in a αβT-iPSC-derived αβT cell (αβT-iαβT cell). In some embodiments, the SOCS1 and CISH genes are genomically disrupted in the αβT-iPSC-derived αβT cell (αβT-iαβT cell). In some embodiments, the SOCS1, CISH, and BIM genes are genomically disrupted in the αβT-iPSC-derived αβT cell (αβT-iαβT cell). In some embodiments, the FAS, SOCS1, CISH, and BIM genes are genomically disrupted in the αβT-iPSC-derived αβT cell (αβT-iαβT cell). In some embodiments, the β2M, CIITA, SOCS1, CISH, and BIM genes are genomically disrupted in the αβT-iPSC-derived αβT cell (αβT-iαβT cell). In some embodiments, the FAS, B2M, CIITA, SOCS1, CISH, and BIM genes are genomically disrupted in the αβT-iPSC-derived αβT cell (αβT-iαβT cell).

In some embodiments, one or more of the SOCS1, CISH, BIM, FAS, B2M, and CIITA genes are genomically disrupted in an iHSC. In some embodiments, the SOCS1 and CISH genes are genomically disrupted the iHSC. In some embodiments, the SOCS1, CISH, and BIM genes are genomically disrupted in the iHSC. In some embodiments, the FAS, SOCS1, CISH, and BIM genes are genomically disrupted in the iHSC. In some embodiments, the β2M, CIITA, SOCS1, CISH, and BIM genes are genomically disrupted in the iHSC. In some embodiments, the FAS, β2M, CIITA, SOCS1, CISH, and BIM genes are genomically disrupted in the iHSC.

In some embodiments, one or more of the SOCS1, CISH, BIM, FAS, B2M, and CIITA genes are genomically disrupted in a αβT-iHSC. In some embodiments, the SOCS1 and CISH genes are genomically disrupted the αβT-iHSC. In some embodiments, the SOCS1, CISH, and BIM genes are genomically disrupted in the αβT-iHSC. In some embodiments, the FAS, SOCS1, CISH, and BIM genes are genomically disrupted in the αβT-iHSC. In some embodiments, the β2M, CIITA, SOCS1, CISH, and BIM genes are genomically disrupted in the αβT-iHSC. In some embodiments, the FAS, B2M, CIITA, SOCS1, CISH, and BIM genes are genomically disrupted in the αβT-iHSC.

In some embodiments, one or more of the SOCS1, CISH, BIM, FAS, B2M, and CIITA genes are genomically disrupted in an iCLP cell. In some embodiments, the SOCS1 and CISH genes are genomically disrupted the iCLP cell. In some embodiments, the SOCS1, CISH, and BIM genes are genomically disrupted in the iCLP cell. In some embodiments, the FAS, SOCS1, CISH, and BIM genes are genomically disrupted in the iCLP cell. In some embodiments, the β2M, CIITA, SOCS1, CISH, and BIM genes are genomically disrupted in the iCLP cell. In some embodiments, the FAS, B2M, CIITA, SOCS1, CISH, and BIM genes are genomically disrupted in the iCLP cell.

In some embodiments, one or more of the SOCS1, CISH, BIM, FAS, B2M, and CIITA genes are genomically disrupted in a αβT-iCLP cell. In some embodiments, the SOCS1 and CISH genes are genomically disrupted the αβT-iCLP cell. In some embodiments, the SOCS1, CISH, and BIM genes are genomically disrupted in the αβT-iCLP cell. In some embodiments, the FAS, SOCS1, CISH, and BIM genes are genomically disrupted in the αβT-iCLP cell. In some embodiments, the β2M, CIITA, SOCS1, CISH, and BIM genes are genomically disrupted in the αβT-iCLP cell. In some embodiments, the FAS, B2M, CIITA, SOCS1, CISH, and BIM genes are genomically disrupted in the αβT-iCLP cell.

In some embodiments, the SOCS1 and CISH genes are genomically disrupted in the αβT-iPSC cell, and the FAS, B2M, CIITA, and/or BIM gene is genomically disrupted in any one of T-iαβT, immature T-iαβT cell, αβT-iHSC, or αβT-iCLP cell.

In some embodiments, the SOCS1 and CISH genes are genomically disrupted in the immature T-iαβT cell, and the FAS, B2M, CIITA, and/or BIM gene is genomically disrupted in any one of αβT-iPSC, T-iαβT, αβT-iHSC, or αβT-iCLP cell.

In some embodiments, the SOCS1 and CISH genes are genomically disrupted in the T-iαβT cell, and the FAS, B2M, CIITA, and/or BIM gene is genomically disrupted in any one of αβT-iPSC, immature T-iαβT cell, αβT-iHSC, or αβT-iCLP cell.

In some embodiments, the SOCS1 and CISH genes are genomically disrupted in the αβT-iHSC, and the FAS, B2M, CIITA, and/or BIM gene is genomically disrupted in any one of αβT-iPSC, immature T-iαβT cell, T-iαβT, or αβT-iCLP cell.

In some embodiments, the SOCS1 and CISH genes are genomically disrupted in the αβT-iCLP cell, and the FAS, B2M, CIITA, and/or BIM gene is genomically disrupted in any one of αβT-iPSC, immature T-iαβT cell, T-iαβT, or αβT-iHSC.

In some embodiments, the exogenous polynucleotide encoding B2M-HLA-E is incorporated into a β2M locus of an iPSC (e.g., αβT-iPSC) and/or one or more exogenous polynucleotides encoding a signal converter are incorporated into an iPSC (e.g., αβT-iPSC).

In some embodiments, the cell is mammalian. In some embodiments, the cell is human cell.

In some embodiments, the cell is in vivo, in vitro, ex vivo, or in a human subject.

Also encompassed are populations of cells comprising one or more modified cells described herein.

In some embodiments, the cells are stored for a length of time before use. In some embodiments, the time is weeks or months. In some embodiments, the time is months or years.

In some embodiments, the cells are stored frozen in liquid nitrogen (−196 degrees Celsius).

### A. Suppressor of Cytokine Signaling 1 (SOCS1) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous suppressor of cytokine signaling 1 (SOCS1) gene. In some embodiments, the modified cell is a modified T-iαβT cell comprising a genomic disruption of the endogenous SOCS1 gene. In some embodiments, the modified cell is a αβT-cell that is differentiated from a modified (e.g., alone or with any one or more genomic disruptions in CISH, BIM, FAS, B2M, CIITA, TRAC and TRBC1/2, and/or any one or more incorporations of B2M-HLA-E and signal converter and a suicide switch) or unmodified αβT-iPSC, modified or unmodified T-iHSC, modified or unmodified T-iCLP, or modified or unmodified immature T-iαβT cell. In some embodiments, the modified cell is from a primary αβT-cell.

In some embodiments, the modified cell comprises a genomic disruption in an endogenous SOCS1 gene that suppresses or eliminates expression of the SOCS1 gene relative to an unmodified control. In some embodiments, the genomic disruption in the SOCS1 gene is an inactivating nucleic acid mutation (e.g., knock out). In some embodiments, the inactivating nucleic acid mutation in the SOCS1 gene is an insertion, deletion, substitution, or any combination thereof. As used herein, “suppresses or eliminates” means any reduction in the level of a functional protein expressed from a gene as compared to a non-modified control. As used herein, “inactivating nucleic acid mutation” means any nucleic acid mutation causing any reduction in the level of a functional protein expressed from a gene as compared to a non-modified control. In some embodiments, the genomic disruption in the SOCS1 gene is an endonuclease-mediated insertion-deletion (indel). In some embodiments, the inactivating nucleic acid mutation in the SOCS1 gene is an endonuclease-mediated indel, such as a targeted knockout obtainable by a CRISPR-Cas system (e.g., Cas9, Cas12 or MAD7) designed to target SOCS1.

In some embodiments, the genomic disruption of the endogenous SOCS1 gene is in exon 2. In some embodiments, the genomic disruption is an inactivating nucleic acid mutation in exon 2. In some embodiments, the genomic disruption is within a target sequence in SOCS1 that comprises the nucleotide sequence of any one of SEQ ID NO: 17, SEQ ID NO: 23, or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iαβT-cell) can be performed using various methods. For example, the relative expression of the SOCS1 gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### B. Cytokine-Inducible SH2-Containing Protein (CISH) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous cytokine-inducible sh2-containing protein (CISH) gene. In some embodiments, the modified cell is a modified T-iαβT cell comprising a genomic disruption of the endogenous CISH gene. In some embodiments, the modified cell is a αβT-cell that is differentiated from a modified (e.g., alone or with any one or more genomic disruptions in SOCS1, BIM, FAS, B2M, CIITA, TRAC and TRBC1/2, and/or any one or more incorporations of B2M-HLA-E and signal converter and a suicide switch) or unmodified αβT-iPSC, a modified or unmodified T-iHSC, a modified or unmodified T-iCLP, or a modified or unmodified immature T-iαβT cell. In some embodiments, the modified cell is from a primary αβT-cell.

In some embodiments, the cell comprises a genomic disruption in an endogenous CISH gene that suppresses or eliminates expression of the CISH gene relative to an unmodified control. In some embodiments, the genomic disruption in the CISH gene is an inactivating nucleic acid mutation (e.g., knock out). In some embodiments, the inactivating nucleic acid mutation in the CISH gene is an insertion, deletion, substitution, or any combination thereof. In some embodiments, the genomic disruption in the CISH gene is an endonuclease-mediated indel. In some embodiments, the inactivating nucleic acid mutation in the CISH gene is an endonuclease-mediated indel, such as a targeted knockout obtainable by a CRISPR-Cas system (e.g., Cas9, Cas12 or MAD7) designed to target CISH.

In some embodiments, the genomic disruption of the endogenous CISH gene is in exon 3, exon 4, or a combination thereof. In some embodiments, the genomic disruption is an inactivating nucleic acid mutation in exon 3, exon 4, or any combination thereof. In some embodiments, the genomic disruption is within a target sequence in the CISH gene that comprises the nucleotide sequence of any one of SEQ ID NO: 15, SEQ ID NO: 16 or SEQ ID NO: 24 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

Assays for determining the relative expression of a gene or function of a protein encoded by the gene in modified cell (e.g., T-iαβT-cell) can be performed using various methods. For example, the relative expression of a CISH gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using qPCR, dPCR, ddPCR, or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### C. Bcl-2 Interacting Mediator of Cell Death (BIM) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous Bcl-2 interacting mediator of cell death (BIM) gene. In some embodiments, the modified cell is a modified T-iαβT cell comprising a genomic disruption of the endogenous BIM gene. In some embodiments, the modified cell is a αβT-cell that is differentiated from a modified (e.g., alone or with any one or more genomic disruptions in SOCS1, CISH, FAS, B2M, CIITA, TRAC and TRBC1/2, and/or any one or more incorporations of B2M-HLA-E and signal converter and a suicide switch) or unmodified αβT-iPSC, modified or unmodified T-iHSC, modified or unmodified T-iCLP, or modified or unmodified immature T-iαβT cell. In some embodiments, the modified cell is from a primary αβT-cell.

In some embodiments, the genomic disruption in the BIM gene is an inactivating nucleic acid mutation (e.g., knock out). In some embodiments, the inactivating nucleic acid mutation in the BIM gene is an insertion, deletion, substitution, or any combination thereof. In some embodiments, the genomic disruption in the BIM gene is an endonuclease-mediated indel. In some embodiments, the inactivating nucleic acid mutation in the BIM gene is an endonuclease-mediated indel such as a targeted knockout obtainable by a CRISPR-Cas system (e.g., Cas9, Cas12 or MAD7) designed to target BIM.

In some embodiments, the genomic disruption of the endogenous BIM gene is in exon 2A. In some embodiments, the genomic disruption is an inactivating nucleic acid mutation in exon 2A. In some embodiments, the genomic disruption of the endogenous BIM gene in the modified cell reduces the expression and/or function of all splice variant of BIM, BIM_{EL}, BIM_{L} and BIM_{S}, relative to an unmodified control.

In some embodiments, the genomic disruption is within a target sequence in BIM that comprises the nucleotide sequence of any one of SEQ ID NO: 18, SEQ ID NO: 19, or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

In some embodiments, the genomic disruption of the endogenous BIM gene is in exon 2C. In some embodiments, the genomic disruption is an inactivating nucleic acid mutation in exon 2C.

In some embodiments, the genomic disruption of the endogenous BIM gene in the modified cell reduces the expression and/or function of a splice variant of BIM, BIM_{EL}, relative to an unmodified control. In some embodiments, the genomic disruption of the endogenous BIM gene reduces the expression and/or function of a splice variant of BIM, BIM_{L}, relative to an unmodified control. In some embodiments, the genomic disruption of the endogenous BIM gene reduces the expression and/or function of a splice variants BIM_{EL} and BIM_{L} relative to an unmodified control.

In some embodiments, the genomic disruption of the endogenous BIM gene in the modified cell retains the expression and/or function of a splice variant of BIM, BIM_{S}, relative to an unmodified control.

In some embodiments, the genomic disruption of the endogenous BIM gene reduces the expression and/or function of a splice variants BIM_{EL} and BIM_{L}, while retaining the expression and/or function of a splice variant of BIM, BIM_{S}, relative to an unmodified control.

In some embodiments, the genomic disruption is within a target sequence in BIM that comprises the nucleotide sequence of any one of SEQ ID NO: 14, SEQ ID NO: 20, SEQ ID NO: 21, or SEQ ID NO: 22 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein in a modified cell can be performed using various methods. Similarly, assays for determining the relative expression or function of a splice variant can be performed using various methods. For example, the relative expression of BIM_{S}, BIM_{EL} and BIM_{L} in a modified cell (e.g., T-iαβT-cell) relative to an unmodified control (e.g., an unmodified T-iαβT-cell) can be determined using qPCR, dPCR, ddPCR, or nucleotide sequencing (e.g., next generation sequencing). Additionally, the relative expression of BIM_{S}, BIM_{EL} and BIM_{L} in a modified αβT-cell (e.g., T-iαβT-cell) relative to each other in the modified αβT-cell (e.g., T-iαβT-cell) can be determined using qPCR, dPCR, ddPCR, or nucleotide sequencing (e.g., next generation sequencing).

### D. FAS Cell Surface Death Receptor (FAS) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous FAS cell surface death receptor (FAS) gene. In some embodiments, the modified cell is a modified T-iαβT cell comprising a genomic disruption of the endogenous FAS gene. In some embodiments, the modified cell is a αβT-cell that is differentiated from a modified (e.g., with any one or more genomic disruptions in SOCS1, CISH, BIM, B2M, CIITA, TRAC and TRBC1/2, and/or any one or more incorporations of B2M-HLA-E and signal converter and a suicide switch) or unmodified αβT-iPSC, modified or unmodified T-iHSC, modified or unmodified T-iCLP, or modified or unmodified immature T-iαβT cell. In some embodiments, the genomic disruptions are of at least two or all three of SOCS1, CISH, and BIM, and further in FAS, and optionally further in CIITA and/or β2M. In some embodiments, the modified cell is from a primary αβT-cell.

In some embodiments, the modified cell comprises a genomic disruption in an endogenous FAS gene that suppresses or eliminates expression of the FAS gene relative to an unmodified control. In some embodiments, the genomic disruption in the FAS gene is an inactivating nucleic acid mutation (e.g., knock out). In some embodiments, the inactivating nucleic acid mutation in the FAS gene is an insertion, deletion, substitution, or any combination thereof. In some embodiments, the genomic disruption in the FAS gene is an endonuclease-mediated indel. In some embodiments, the inactivating nucleic acid mutation in the FAS gene is an endonuclease-mediated indel, such as a targeted knockout obtainable by a CRISPR-Cas system (e.g., Cas9, Cas12 or MAD7) designed to target FAS.

In some embodiments, the genomic disruption of the endogenous FAS gene is in exon 1. In some embodiments, the genomic disruption is an inactivating nucleic acid mutation in exon 1. In some embodiments, the genomic disruption is within a target sequence in FAS that comprises the nucleotide sequence of SEQ ID NO: 62 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iαβT-cell) can be performed using various methods. For example, the relative expression of the FAS gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### E. β-2-Microglobulin (B2M) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous β-2-Microglobulin (B2M) gene. In some embodiments, the modified cell is a modified T-iαβT cell comprising a genomic disruption of the endogenous β2M gene. In some embodiments, the modified cell is a αβT-cell that is differentiated from a modified (e.g., with any one or more genomic disruptions in SOCS1, CISH, BIM, FAS, CIITA, TRAC and TRBC1/2, and/or any one or more incorporations of B2M-HLA-E and signal converter and a suicide switch) or unmodified αβT-iPSC, modified or unmodified T-iHSC, modified or unmodified T-iCLP, or modified or unmodified immature T-iαβT cell. In some embodiments, the genomic disruptions are of at least two or all three of SOCS1, CISH, and BIM, and further in CIITA and/or B2M, and optionally further in FAS. In some embodiments, the modified cell is from a primary αβT-cell.

In some embodiments, the modified cell comprises a genomic disruption in an endogenous B2M gene that suppresses or eliminates expression of the B2M gene relative to an unmodified control. In some embodiments, the genomic disruption in the B2M gene is an inactivating nucleic acid mutation (e.g., knock out). In some embodiments, the inactivating nucleic acid mutation in the B2M gene is an insertion, deletion, substitution, or any combination thereof. In some embodiments, the genomic disruption in the B2M gene is an endonuclease-mediated indel. In some embodiments, the inactivating nucleic acid mutation in the B2M gene is an endonuclease-mediated indel, such as a targeted knockout obtainable by a CRISPR-Cas system (e.g., Cas9, Cas12 or MAD7) designed to target B2M.

In some embodiments, the genomic disruption of the endogenous B2M gene is in exon 2. In some embodiments, the genomic disruption is an inactivating nucleic acid mutation in exon 2. In some embodiments, the genomic disruption is within a target sequence in B2M that comprises the nucleotide sequence of SEQ ID NO: 56 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iαβT-cell) can be performed using various methods. For example, the relative expression of the β2M gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### F. Class II Transactivator (CIITA) Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six, or seven or more genomic disruptions and/or one, two, three, or four or more of exogenous polynucleotide incorporations, one of which is a genomic disruption of the endogenous Class II transactivator (CIITA) gene. In some embodiments, the modified cell is a modified T-iαβT cell comprising a genomic disruption of the endogenous CIITA gene. In some embodiments, the modified cell is a αβT-cell that is differentiated from a modified (e.g., with any one or more genomic disruptions in SOCIS1, CISH, BIM, FAS, B2M, TRAC and TRBC1/2, and/or any one or more incorporations of B2M-HLA-E and signal converter and a suicide switch) or unmodified αβT-iPSC, modified or unmodified T-iHSC, modified or unmodified T-iCLP, or modified or unmodified immature T-iαβT cell. In some embodiments, the genomic disruptions are of at least two or all three of SOCS1, CISH, and BIM, and further in CIITA and/or B2M, and optionally further in FAS. In some embodiments, the modified cell is from a primary αβT-cell.

In some embodiments, the modified cell comprises a genomic disruption in an endogenous CIITA gene that suppresses or eliminates expression of the CIITA gene relative to an unmodified control. In some embodiments, the genomic disruption in the CIITA gene is an inactivating nucleic acid mutation (e.g., knock out). In some embodiments, the inactivating nucleic acid mutation in the CIITA gene is an insertion, deletion, substitution, or any combination thereof. In some embodiments, the genomic disruption in the CIITA gene is an endonuclease-mediated indel. In some embodiments, the inactivating nucleic acid mutation in the CIITA gene is an endonuclease-mediated indel, such as a targeted knockout obtainable by a CRISPR-Cas system (e.g., Cas9, Cas12 or MAD7) designed to target CIITA.

In some embodiments, the genomic disruption of the endogenous CIITA gene is in exon 3. In some embodiments, the genomic disruption is an inactivating nucleic acid mutation in exon 3. In some embodiments, the genomic disruption is within a target sequence in CIITA that comprises the nucleotide sequence of SEQ ID NO: 59 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iαβT-cell) can be performed using various methods. For example, the relative expression of the CIITA gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

G. TRAC and/or TRBC1/2 Genomic Disruption

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two or three or four or more of exogenous polynucleotide incorporations, one of which is a genomic disruption in TRAC gene and/or a genomic disruption in TRBC1/2 gene. In some embodiments, the modified cell is a modified T-iαβT cell comprising a genomic disruption in the endogenous TRAC gene and/or a genomic disruption in the TRBC1/2 gene. In some embodiments, the modified cell is a modified T-iαβT cell comprising a genomic disruption in the endogenous TRAC gene and a genomic disruption in the TRBC1/2 gene. In some embodiments, the modified cell is a modified T-iαβT cell comprising a genomic disruption in the endogenous TRAC gene and a genomic disruption in the TRBC1 and TRBC2 gene. In some embodiments, the modified cell is a αβT-cell that is differentiated from a modified αβT-iPSC (e.g., with any one or more genomic disruptions in SOCIS1, CISH, BIM, FAS, B2M, and CIITA, and/or any one or more incorporations of B2M-HLA-E and signal converter) or unmodified αβT-iPSC, modified or unmodified T-iHSC, modified or unmodified T-iCLP, or modified or unmodified immature T-iαβT cell. In some embodiments, the modified cell is from a primary αβT-cell.

In some embodiments, the modified cell comprises a genomic disruption in an endogenous TRAC gene that suppresses or eliminates expression of the TRAC gene relative to an unmodified control and/or a genomic disruption in an endogenous TRBC1/2 gene that suppresses or eliminates expression of a functional TRBC1/2 protein. In some embodiments, the modified cell comprises a genomic disruption in an endogenous TRAC gene that suppresses or eliminates expression of the TRAC gene relative to an unmodified control and a genomic disruption in an endogenous TRBC1 and TRBC2 gene that suppresses or eliminates expression of the functional TRBC1 and TRBC2 protein. In some embodiments, the genomic disruption in the TRAC gene and/or TRBC1/2 gene is an inactivating nucleic acid mutation (e.g., knock out). In some embodiments, the inactivating nucleic acid mutation in the TRAC gene and/or TRBC1/2 gene is an insertion, deletion, substitution, or any combination thereof. In some embodiments, the genomic disruption in the TRAC gene and/or TRBC1/2 gene is an endonuclease-mediated indel. In some embodiments, the inactivating nucleic acid mutation in the TRAC gene and/or TRBC1/2 gene is an endonuclease-mediated indel, such as a targeted knockout obtainable by a CRISPR-Cas system (e.g., Cas9, Cas12 or MAD7) designed to target TRAC and/or TRBC1/2 gene.

Assays for determining the relative expression of a gene or function of a protein encoded by the gene in a modified cell (e.g., T-iαβT-cell) can be performed using various methods. For example, the relative expression of the SOCIS1, CISH, BIM, FAS, B2M, CIITA, TRAC gene, or TRBC1/2 gene in a modified αβT-cell (e.g., T-iαβT-cell) relative to an unmodified αβT-cell (e.g., an unmodified T-iαβT-cell) or relative to the αβT-cell (e.g., T-iαβT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### H. B2M-HLA-E Incorporation

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two, three or four or more of exogenous polynucleotide incorporations, one of which is an incorporation of B2M-HLA-E. In some embodiments, the modified cell is a modified T-iαβT cell comprising an exogenous polynucleotide encoding B2M-HLA-E. In some embodiments, the modified cell is a αβT-cell that is differentiated from a modified iPSC (e.g., alone or with any one or more genomic disruptions in SOCS1, CISH, BIM, FAS, B2M, CIITA, TRAC and TRBC1/2, and/or any one or more incorporations of signal converter and a suicide switch). In some embodiments, the modified cell is from a primary αβT-cell.

In some embodiments, the modified cell comprises an exogenous polynucleotide encoding B2M-HLA-E which results in the expression of B2M-HLA-E relative to an unmodified control. In some embodiments, the exogenous polynucleotide encoding B2M-HLA-E is incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the exogenous polynucleotide encoding B2M-HLA-E is inserted into a B2M locus. In some embodiments, the exogenous polynucleotide encoding B2M-HLA-E is incorporated to the modified cell by a lentiviral vector.

In some embodiments, B2M-HLA-E is a fusion protein comprising at least a portion of B2M fused with at least of a portion of HLA-E. In some embodiments, the B2M-HLA-E comprises an amino acid sequence of SEQ ID NO: 81. In some embodiments, the B2M-HLA-E consists essentially of or consists of an amino acid sequence of SEQ ID NO: 81.

In some embodiments, the CAG promoter was used to drive the expression from the exogenous polynucleotide encoding B2M-HLA-E.

### I. Signal Converter

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two, three or four or more of exogenous polynucleotide incorporations, one of which is an incorporation of a signal converter. In some embodiments, the modified cell is a modified T-iαβT cell comprising one or more exogenous polynucleotides encoding a signal converter. In some embodiments, the modified cell is a αβT-cell that is differentiated from a modified iPSC (e.g., alone or with any one or more genomic disruptions in SOCS1, CISH, BIM, FAS, B2M, CIITA, TRAC and TRBC1/2, and/or an incorporation of B2M-HLA-E and/or a suicide switch). In some embodiments, the modified cell is from a primary αβT-cell.

In some embodiments, the signal converter is a chimeric structure of two or more different cytokine receptors, wherein the extracellular structure of the signal converter is from one cytokine receptor A and the intracellular structure of the signal converter is from another cytokine receptor B, the binding of cytokine A with the extracellular structure of the cytokine receptor A will transfer, transit or convert the intracellular signaling via the intracellular structure of the cytokine receptor B, which activates or improves the signal transducer and activator of transcription (STAT) signaling in the cell which is not, little or less activated by the natural cytokine receptor A when binding with cytokine A. In some embodiments, the extracellular structure of the signal converter comprises one or more extracellular domains, from same or different cytokine receptor, or from one or more cytokine receptors. In some embodiments, the intracellular structure of the signal converter comprises one or more intracellular domains, from same or different cytokine receptor, or from one or more cytokine receptors.

In some embodiment, the extracellular structure of the signal converter is from interferon gamma receptor. In some embodiment, the intracellular structure of the signal converter is from interleukin-2 receptor. In some embodiments, the extracellular structure of the signal converter and the intracellular structure is linked via transmembrane structure, wherein the transmembrane structure comprises transmembrane domain from same or different cytokine receptors where the extracellular structure of the signal converter and the intracellular structure are from. In some embodiments, the transmembrane structure comprises transmembrane domain from same cytokine receptors where the extracellular structure of the signal converter or the intracellular structure is from. In some embodiments, the signal converter comprises membrane-proximal region between the ECD and the intracellular domain, said membrane-proximal region is from the same or different cytokine receptors where the extracellular structure of the signal converter and the intracellular structure is from. In some embodiments, the membrane-proximal region is from the same cytokine receptor where the extracellular domain of the signal converter is from, or and the same cytokine receptor where the intracellular domain of the signal converter is from. In some embodiments, the signal converter further comprises a signal peptide that enables trafficking of the signal converter to the cell membrane. In some embodiments, the signal peptide is removed from the signal converter after localization to the membrane.

### Signal Converter Comprising ECD of IFNGR and ICD of IL2R

In some embodiment, the extracellular structure of the signal converter comprises both the extracellular domain (ECD) of interferon gamma receptor 1 (IFNGR1) and the extracellular domain (ECD) of interferon gamma receptor 2 (IFNGR2). In some embodiments, the intracellular structure of the signal converter comprises the intracellular domain of interleukin-2 receptor subunit gamma (IL2RG) and the intracellular domain of interleukin-2 receptor subunit beta (IL2RB), wherein one ECD of IFNGR1 and IFNGR2 is linked or fused with one ICD of IL2RG and IL2RB to form a first fusion protein subunit, and the other ECD is linked or fused with the other ICD to form a second fusion protein subunit. In some embodiments, the signal converter further comprises transmembrane domains of IFNGR1 and IFNGR2, or the signal converter further comprises transmembrane domains of IL2RG and IL2RB. In some embodiments, the signal converter further comprises membrane-proximal regions of IFNGR1 and IFNGR2, or the signal converter further comprises membrane-proximal regions of IL2RG and IL2RB. In some embodiments, one of the IFNGR1 or IFNGR2 extracellular domain is fused sequentially with transmembrane domain, and intracellular domain of IL2RG to form the first fusion protein subunit, the other IFNGR2 or IFNGR1 extracellular domain is fused sequentially with transmembrane domain, and intracellular domain of IL2RB to form the second fusion protein subunit. In some embodiment, one of the IFNGR1 or IFNGR2 extracellular domain is fused sequentially with membrane-proximal region, transmembrane domain, and intracellular domain of IL2RG to form the first fusion protein subunit, the other IFNGR2 or IFNGR1 extracellular domain is fused sequentially with membrane-proximal region, transmembrane domain, and intracellular domain of IL2RB to form the second fusion protein subunit. In some embodiments, the first fusion protein subunit and the second fusion protein subunit of the signal converter further comprise a signal peptide.

In some embodiments, the signal peptide of the first fusion protein subunit comprises the amino acid sequence of SEQ ID NO: 73 or SEQ ID NO: 74, and the signal peptide of the second fusion protein subunit comprises the amino acid sequence of SEQ ID NO: 73 or SEQ ID NO: 74.

In some embodiments, the amino acid sequence of the signal peptide of the first fusion protein subunit is SEQ ID NO: 73, and the amino acid sequence of the signal peptide of the second fusion protein subunit is SEQ ID NO: 74. In some embodiments, the amino acid sequence of extracellular domain of the first fusion protein subunit is SEQ ID NO: 63, and the amino acid sequence of extracellular domain of the second fusion protein subunit is SEQ ID NO: 64. In some embodiments, the amino acid sequence of intracellular domain of the first fusion protein subunit is SEQ ID NO: 69, and the amino acid sequence of intracellular domain of the second fusion protein subunit is SEQ ID NO: 70. In some embodiments, the amino acid sequence of transmembrane domain of the first fusion protein subunit is SEQ ID NO: 67, and the amino acid sequence of transmembrane domain of the second fusion protein subunit is SEQ ID NO: 68. The amino acid sequence of membrane-proximal region of the first fusion protein subunit is SEQ ID NO: 65, and the amino acid sequence of membrane-proximal region of the second fusion protein subunit is SEQ ID NO: 66.

In some embodiments, the present disclosure provides a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB). In some embodiments, the first fusion protein subunit of the signal converter comprises an amino acid sequence of SEQ ID NO: 71, and the second fusion protein subunit of the signal converter comprises an amino acid sequence of SEQ ID NO: 72. In some embodiments, the first fusion protein subunit of the signal converter consists essentially of or consists of an amino acid sequence of SEQ ID NO: 71, and the second fusion protein subunit of the signal converter consists essentially of or consists of an amino acid sequence of SEQ ID NO: 72. In some embodiments, the first exogenous polynucleotide encoding an amino acid sequence comprising SEQ ID NO: 75 (the first fusion protein subunit with signal peptide), and the second exogenous polynucleotide encoding an amino acid sequence comprising SEQ ID NO: 76 ((the second fusion protein subunit with signal peptide)).

In some embodiments, the modified cell comprises one or more exogenous polynucleotides encoding a signal converter, which results in the expression of signal converter relative to an unmodified control. In some embodiments, the one or more exogenous polynucleotides encoding the signal converter are incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, one or more exogenous polynucleotides encoding the signal converter are incorporated to the modified cell by a lentiviral vector.

In some embodiments, the first exogenous polynucleotide encoding the first fusion protein subunit of the signal converter is inserted into a SOCS1 locus. In some embodiments, the second exogenous polynucleotide encoding the second fusion protein subunit of the signal converter is inserted into a ROSA26 locus.

In some embodiments, the first exogenous polynucleotide encoding the first fusion protein subunit of the signal converter is inserted into a ROSA26 locus. In some embodiments, the second exogenous polynucleotide encoding the second fusion protein subunit of the signal converter is inserted into a SOCS1 locus.

In some embodiments, the CAG promoter was used to drive the expression from the first exogenous polynucleotide encoding the first fusion protein subunit and the second exogenous polynucleotide encoding the second fusion protein subunit.

### J. Suicide Switch Incorporation

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two, three or four or more of exogenous polynucleotide incorporations, one of which is an incorporation of B2M-HLA-E. In some embodiments, the modified cell is a modified T-iαβT cell comprising an exogenous polynucleotide encoding B2M-HLA-E. In some embodiments, the modified cell is a αβT-cell that is differentiated from a modified iPSC (e.g., alone or with any one or more genomic disruptions in SOCS1, CISH, BIM, FAS, B2M, CIITA, TRAC and TRBC1/2, and/or any one or more incorporations of B2M-HLA-E and signal converter). In some embodiments, the modified cell is from a primary αβT-cell.

In some embodiments, the exogenous polynucleotide encoding a suicide switch is incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the exogenous polynucleotide encoding the suicide switch is inserted into a Rogi2 locus. In some embodiments, the exogenous polynucleotide encoding the suicide switch is inserted into a FAS locus. In some embodiments, the exogenous polynucleotide encoding a suicide switch is incorporated to the modified cell by a lentiviral vector.

In some embodiments, the suicide switch comprises a herpes simplex virus thymidine kinase (HSV-tk) polypeptide or a functional variant thereof capable of inducing cell death upon administration of Ganciclovir (GCV). In some embodiments, the suicide switch comprises an amino acid sequence of SEQ ID NO: 85. In some embodiments, the suicide switch consists essentially of or consists of an amino acid sequence of SEQ ID NO: 85.

### K. Single, Double, Triple, Quadruple, Quintuple, and Sextuple or More Genomic Disruptions

The present disclosure provides single-gene, double-gene, triple-gene, quadruple-gene, quintuple-gene, or sextuple-gene modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one or more genomic disruptions in the following genes: the endogenous SOCS1 gene, CISH gene, BIM gene, FAS gene, B2M gene, and CIITA gene. The present disclosure provides double-gene, triple-gene, quadruple-gene, quintuple-gene, or sextuple-gene modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising a genomic disruption of the endogenous SOCS1 and CISH genes, optionally a BIM gene, optionally a FAS gene, optionally a B2M gene, and, optionally a CIITA gene. In some embodiments, the modified cell is a T-iαβT cell comprising a genomic disruption of the endogenous SOCS1 and CISH genes. In some embodiments, the modified cell is a T-iαβT cell comprising a genomic disruption of the endogenous SOCS1, CISH, and BIM genes. In some embodiments, the modified cell is a T-iαβT cell comprising a genomic disruption of the endogenous SOCS1, CISH, BIM, and FAS genes. In some embodiments, the modified cell is a T-iαβT cell comprising a genomic disruption of the endogenous SOCS1, CISH, BIM, B2M, and CIITA genes. In some embodiments, the modified cell is a T-iαβT cell comprising a genomic disruption of the endogenous SOCS1, CISH, BIM, FAS, B2M, and CIITA genes. In some embodiments, the modified cell is differentiated from a αβT-iPSC, T-iHSC, T-iCLP, or immature T-iαβT cell, where the modifications can occur at any of these cell development stages and be carried through to αβT maturity, or wherein the modifications can occur at αβT maturity, i.e., at the T-iαβT stage.

In some embodiments, the modified cell comprises a genomic disruption in an endogenous SOCS1 and CISH genes that suppresses or eliminates expression of the SOCS1 and CISH genes.

The present disclosure provides modified iPSC-derived αβT-cells (and their precursors (e.g., αβT-iPSC, αβT-iHSC, αβT-iCLP)) comprising a genomic disruption in an endogenous SOCS1 gene that suppresses or eliminates expression of SOCS1, a genomic disruption in an endogenous CISH gene that suppresses or eliminates expression of CISH, and, optionally, a genomic disruption in an endogenous BIM gene that suppresses or eliminates expression of BIM.

The present disclosure provides modified iPSC-derived αβT-cells (and their precursors (e.g., αβT-iPSC, αβT-iHSC, αβT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, and, optionally a genomic disruption in an endogenous FAS gene that suppresses or eliminates expression of FAS.

The present disclosure provides modified iPSC-derived αβT-cells (and their precursors (e.g., αβT-iPSC, αβT-iHSC, αβT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, optionally a genomic disruption in an endogenous β2M gene that suppresses or eliminates expression of β2M, and, optionally a genomic disruption in an endogenous CIITA gene that suppresses or eliminates expression of CIITA.

The present disclosure provides modified iPSC-derived αβT-cells (and their precursors (e.g., αβT-iPSC, αβT-iHSC, αβT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, optionally a genomic disruption in an endogenous FAS gene that suppresses or eliminates expression of FAS, optionally a genomic disruption in an endogenous β2M gene that suppresses or eliminates expression of β2M, and, optionally a genomic disruption in an endogenous CIITA gene that suppresses or eliminates expression of CIITA.

In some embodiments, the expression and/or function of BIM_{EL} and BIM_{L} splice variants are reduced in the modified cell relative to that in an unmodified control. In some embodiments, the expression and/or function of BIM_{S} splice variant is retained in the modified cell.

In some embodiments, the single-gene, double-gene, triple-gene, quadruple-gene, quintuple-gene, or sextuple-gene modified αβT-cells could further comprise a genomic disruption of the TRAC gene and/or TRBC1/2 gene.

In some embodiments, the genomic disruption is made by administering guide RNAs to the cell, optionally wherein the guide RNAs are single-guide RNAs (sgRNAs), and wherein an endonuclease or a nucleic acid encoding an endonuclease is also administered to the cell. Further information on the guide RNA and endonuclease could be found in other sections of the present disclosure, e.g., Section V: Gene editing systems.

Also encompassed are populations of cells comprising the single-, double-, triple-, quadruple-, quintuple-, or sextuple-genomic disruptions described herein.

In some embodiments, the cells are stored for a length of time before use. In some embodiments, the time is weeks or months. In some embodiments, the time is months or years.

In some embodiments, the cells are stored frozen in liquid nitrogen at −196 degrees Celsius.

III. Modified Cells Comprising CAR and/or TCRαβ, e.g., Anti-KRAS G12V TCR

The modified iPSC, αβT cell, or intermediate cell differentiated from an iPSC herein, including Section II, can serve as a platform cell for introduction of an antigen receptor, such as chimeric antigen receptor (CAR) or T cell receptor (TCR).

The modified cells herein, e.g., the modified cells described in Section II, further comprise one or more exogenous polynucleotides encoding a T cell receptor (TCR).

The modified cells herein, e.g., the modified cells described in Section II, further comprise one or more exogenous polynucleotides encoding a chimeric antigen receptor (CAR), to recognize tumor-associated antigens (TAAs) expressed on the surface of cancer cells.

The modified cells herein, e.g., the modified cells described in Section II, further comprise one or more exogenous polynucleotides encoding CAR (e.g., an exogenous polynucleotide encoding anti-CD19 CAR and/or an exogenous polynucleotide encoding anti-CD20 CAR) and/or TCRαβ, e.g., the TCRαβ which specifically binds KRAS G12V (anti-KRAS G12V TCR).

In some embodiments, the modified cells herein, e.g., the modified cells described in Section II, further comprise one or more exogenous polynucleotides encoding TCRαβ, e.g., the TCRαβ which specifically binds KRAS G12V (anti-KRAS G12V TCR).

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two, three or four or more of exogenous polynucleotide incorporations, one of which is an incorporation of anti-KRAS G12V TCR. In some embodiments, the modified cell is a modified T-iαβT cell comprising an exogenous polynucleotide encoding anti-KRAS G12V TCR. In some embodiments, the modified cell is a αβT-cell that is differentiated from a modified iPSC (e.g., with any one or more genomic disruptions in SOCS1, CISH, BIM, FAS, B2M, CIITA, TRAC and TRBC1/2, and/or any one or more incorporations of B2M-HLA-E and signal converter, and CAR). In some embodiments, the modified cell is from a primary αβT-cell.

In some embodiments, the modified cell comprises one or more exogenous polynucleotides encoding anti-KRAS G12V TCR which results in the expression of anti-KRAS G12V TCR relative to an unmodified control. In some embodiments, the one or more exogenous polynucleotides encoding anti-KRAS G12V TCR are incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the one or more exogenous polynucleotides encoding anti-KRAS G12V TCR are inserted into a TRAC locus. In some embodiments, the one or more exogenous polynucleotides encoding anti-KRAS G12V TCR is incorporated to the modified cell by a lentiviral vector.

In some embodiments, anti-KRAS G12V TCR comprises an α chain having the amino acid sequence of SEQ ID NO: 79 and a β chain having the amino acid sequence of SEQ ID NO: 80. In some embodiments, anti-KRAS G12V TCR comprises an α chain consisting essentially of or consisting of an amino acid sequence of SEQ ID NO: 79 and a β chain consisting essentially of or consisting of an amino acid sequence of SEQ ID NO: 80.

In some embodiments, anti-KRAS G12V TCR comprises an α chain having the amino acid sequence of SEQ ID NO: 82 and a β chain having the amino acid sequence of SEQ ID NO: 83. In some embodiments, anti-KRAS G12V TCR comprises an α chain consisting essentially of or consisting of an amino acid sequence of SEQ ID NO: 82 and a β chain consisting essentially of or consisting of an amino acid sequence of SEQ ID NO: 83.

In some embodiments, the exogenous TCRαβ (e.g., anti-KRAS G12V TCR) further comprised in the modified cells herein is used to improve the differentiation efficiency from iPSC to αβT when the wide type TCRαβ therein is not functional (e.g., without a functional TRAC; e.g., TRAC KO). In some embodiments, the exogenous TCRαβ (e.g., anti-KRAS G12V TCR) further comprised in the modified cells herein is used to target KRAS G12V on cancer cells.

In some embodiments, the modified cells herein, e.g., the modified cells described in Section II, further comprise an exogenous polynucleotide encoding a chimeric antigen receptor (CAR), e.g., a CAR which specifically binds human CD19 (anti-CD19 CAR), optionally further comprising an exogenous polynucleotide encoding a TCRαβ, e.g., the TCRαβ which specifically binds KRAS G12V (anti-KRAS G12V TCR).

In some embodiments, the modified cells herein, e.g., the modified cells described in Section II, further comprise an exogenous polynucleotide encoding a chimeric antigen receptor (CAR), e.g., a CAR which specifically binds human CD20 (anti-CD20 CAR), optionally further comprising an exogenous polynucleotide encoding a TCRαβ, e.g., the TCRαβ which specifically binds KRAS G12V (anti-KRAS G12V TCR).

In some embodiments, the modified cells herein, e.g., the modified cells described in Section II, further comprise an exogenous polynucleotide encoding a CAR which specifically binds human CD19 (anti-CD19 CAR) and an exogenous polynucleotide encoding a CAR which specifically binds human CD20 (anti-CD20 CAR), optionally further comprising an exogenous polynucleotide encoding a TCRαβ, e.g., the TCRαβ which specifically binds KRAS G12V (anti-KRAS G12V TCR).

The present disclosure provides modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising one, two, three, four, five, six or seven or more genomic disruptions and/or one, two, three or four or more of exogenous polynucleotide incorporations, one of which is an incorporation of anti-CD19 CAR and/or anti-CD20 CAR. In some embodiments, the modified cell is a modified T-iαβT cell comprising an exogenous polynucleotide encoding anti-CD19 CAR and/or anti-CD20 CAR. In some embodiments, the modified cell is a αβT-cell that is differentiated from a modified iPSC (e.g., with any one or more genomic disruptions in SOCS1, CISH, BIM, FAS, B2M, CIITA, TRAC and TRBC1/2, and/or any one or more incorporations of B2M-HLA-E and signal converter, and TCR). In some embodiments, the modified cell is from a primary αβT-cell.

In some embodiments, the modified cell comprises an exogenous polynucleotide encoding anti-CD19 CAR which results in the expression of anti-CD19 CAR relative to an unmodified control. In some embodiments, the exogenous polynucleotide encoding anti-CD19 CAR is incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the exogenous polynucleotide encoding anti-CD19 CAR is inserted into an AAVS1 locus, which is a safe harbor. In some embodiments, the exogenous polynucleotide encoding anti-CD19 CAR is incorporated to the modified cell by a lentiviral vector.

In some embodiments, anti-CD19 CAR comprising an amino acid sequence of SED ID NO: 93. In some embodiments, anti-human CLL1 CAR comprises an amino acid sequence of SED ID NO: 94. In some embodiments, anti-CD19 CAR consists essentially of or consists of an amino acid sequence of SEQ ID NO: 93 or SEQ ID NO: 94.

In some embodiments, the anti-CD19 CAR further comprises a signal peptide (e.g., comprising an amino acid sequence of SEQ ID NO: 87) that enables trafficking of the CAR to the cell membrane. In some embodiments, the signal peptide is removed from the signal converter after localization to the membrane.

In some embodiments, the CAG promoter was used to drive the expression from the exogenous polynucleotide encoding anti-CD19 CAR.

### IV. Functional Enhancement of Proliferation and Killing Activity

In some embodiments, the present disclosure presents modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) with functional enhancement of proliferation and/or tumor killing activity. More specifically, the functional enhancement is achieved by activating or improved activation of signal transducer and activator of transcription (STAT) signaling in the cell. Also disclosed is the method of improving the proliferation and/or tumor killing activity of αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))), by activating or improved activation of signal transducer and activator of transcription (STAT) signaling in the cell.

In some embodiments, the method of functional enhancement of proliferation and/or tumor killing activity is achieved by introducing one or more exogenous polynucleotides encoding a signal converter into the iPSC or αβT-cells.

Further information on the signal converter could be found in other sections of the present disclosure, e.g., Section II (I): Signal converter.

In some embodiments, the amino acid sequence of ECD of the first fusion protein subunit is SEQ ID NO: 63, and the amino acid sequence of ECD of the second fusion protein subunit is SEQ ID NO: 64. In some embodiments, the amino acid sequence of intracellular domain of the first fusion protein subunit is SEQ ID NO: 69, and the amino acid sequence of intracellular domain of the second fusion protein subunit is SEQ ID NO: 70. In some embodiments, the amino acid sequence of transmembrane domain of the first fusion protein subunit is SEQ ID NO: 67, and the amino acid sequence of transmembrane domain of the second fusion protein subunit is SEQ ID NO: 68. The amino acid sequence of membrane-proximal region of the first fusion protein subunit is SEQ ID NO: 65, and the amino acid sequence of membrane-proximal region of the second fusion protein subunit is SEQ ID NO: 66.

In some embodiments, the present disclosure provides the modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) comprising a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB), optionally further comprising an exogenous polynucleotide encoding a chimeric antigen receptor (CAR) which specifically binds human CD19 and/or an exogenous polynucleotide encoding a chimeric antigen receptor (CAR) which specifically binds human CD20, optionally wherein the exogenous polynucleotide encoding the CAR which specifically binds human CD19 is inserted into an AAVS1 locus, optionally wherein the CAR which specifically binds human CD19 comprises, consists essentially of or consists of the amino acid sequence of SEQ ID NO: 93 or SEQ ID NO: 94.

In some embodiments, the first fusion protein subunit of the signal converter comprises an amino acid sequence of SEQ ID NO: 71, and the second fusion protein subunit of the signal converter comprises an amino acid sequence of SEQ ID NO: 72. In some embodiments, the first fusion protein subunit of the signal converter consists essentially of or consists of an amino acid sequence of SEQ ID NO: 71, and the second fusion protein subunit of the signal converter consists essentially of or consists of an amino acid sequence of SEQ ID NO: 72. In some embodiments, the first exogenous polynucleotide encoding an amino acid sequence comprising SEQ ID NO: 75 (the first fusion protein subunit with signal peptide), and the second exogenous polynucleotide encoding an amino acid sequence comprising SEQ ID NO: 76 ((the second fusion protein subunit with signal peptide)).

In some embodiments, the amino acid sequence of the first fusion protein subunit is SEQ ID NO: 71, and the amino acid sequence of the second fusion protein subunit is SEQ ID NO: 72.

The modified αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) with functional enhancement of proliferation and/or tumor killing activity disclosed herein can be previously or further subject to gene editing for suppressing cytokine signaling disclosed in the other sections of this disclosure.

### V. Gene Editing Systems

Disclosed herein are gene editing systems that are optimized to disrupt the genomes of the cells herein (e.g., the αβT cell, iαβT cell, iPSC, iHSC, iCLP cell, or immature iT cell), specifically within the genes of SOCS1; CISH; BIM; SOCS1 and CISH; SOCS1, CISH, and BIM; SOCS1, CISH, and FAS; SOCS1, CISH, BIM, and FAS; β2M, CIITA, CISH, SOCS1, and BIM; β2M, CIITA, CISH, SOCS1, BIM, and FAS.

### A. Clustered Regularly Interspace Short Palindromic Repeats (CRISPR)

In some embodiments, the gene editing system used to modify αβT-cells, e.g., iPSC-derived αβT-cells (and their precursors (e.g., iPSC (e.g., αβT-iPSC), or an intermediate cell differentiated from an iPSC (e.g., αβT-iHSC, αβT-iCLP, and immature T-iαβT cells))) is clustered regularly interspace short palindromic repeats (CRISPR) system comprising one or more guide RNAs and an endonuclease or a nucleic acid encoding a nuclease. The CRISPR gene editing system comprises a guide RNA, including e.g., a single guide RNA (sgRNA) or crRNA, and a nuclease, including e.g., an endonuclease, or a nucleic acid encoding a nuclease, including e.g., a nucleic acid encoding an endonuclease.

In some embodiments, the guide RNA comprises a guide sequence that binds to a target sequence in SOCS1, CISH, BIM, B2M, CIITA, and/or FAS of the cells used herein (e.g., the T-iαβT cell, the αβT-iPSC, T-iHSC, T-iCLP, or immature T-iαβT cell). In some embodiments, the genomic disruption within SOCS1, CISH, BIM, B2M, CIITA, or FAS gene is made by administering a nuclease (e.g., endonuclease), a plurality of nucleases (e.g., endonucleases), or a nucleic acid encoding an endonuclease(s) and a guide RNA or a plurality of guide RNAs to the cells used herein (e.g., the T-iαβT cell, the αβT-iPSC, T-iHSC, T-iCLP, or immature T-iαβT cell).

In some embodiments, the administration of the nuclease and the guide RNA to the cells used herein (e.g., the T-iαβT cell, the αβT-iPSC, T-iHSC, T-iCLP, or immature T-iαβT cell) is performed sequentially in which a guide RNA targeting a first gene (e.g., SOCS1) is administered initially, followed by administration of a guide RNA targeting a second gene (e.g., CISH). In some embodiments, an administration of a guide RNA targeting a third gene (e.g., BIM) can be performed. In some embodiments, an administration of a guide RNA targeting a fourth gene (e.g., FAS) can be performed. In some embodiments, an administration of a guide RNA targeting a fifth gene (e.g., B2M) can be performed. In some embodiments, an administration of a guide RNA targeting a sixth gene (e.g., CIITA) can be performed. The sequential administration of guide RNA targeting a gene can be performed in any order.

Disclosed herein are single guide RNA (sgRNA) or crRNA nucleotide sequences that bind to a target sequence in the SOCS1 gene in which the sgRNA or crRNA nucleotide sequence comprises any one or more of SEQ ID NOs: 5, 29, 11, 37, and a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA or crRNA nucleotide sequences that bind to a target sequence in the CISH gene in which the sgRNA or crRNA nucleotide sequence comprises any one or more of SEQ ID NOs: 3, 27, 4, 28, 12, 38, and a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA or crRNA nucleotide sequences that bind to a target sequence in the BIM gene in which the sgRNA or crRNA nucleotide sequence comprises any one or more of SEQ ID NOs: 2, 26, 6, 32, 7, 33, 8, 34, 9, 35, 10, 36, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the FAS gene in which the sgRNA nucleotide sequence comprises any one or more of SEQ ID NOs: 60, 61, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the β2M gene in which the sgRNA nucleotide sequence comprises any one or more of SEQ ID NOs: 54, 55, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the CIITA gene in which the sgRNA nucleotide sequence comprises any one or more of SEQ ID NOs: 57, 58, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

Disclosed herein is a gene editing system comprising one or more guide RNA (e.g., sgRNA or crRNA) and an endonuclease or a nucleic acid encoding an endonuclease, in which the one or more guide RNA comprises a guide sequence or a sgRNA/crRNA sequence comprising:
(1) any one of SEQ ID NOs: 5, 29, 11, and 37;
(2) any one of SEQ ID NOs: 3, 27, 4, 28, 12, and 38;
(3) any one of SEQ ID NOs: 2, 26, 6, 32, 7, 33, 8, 34, 9, 35, 10 and 36;
(4) any one of SEQ ID NOs: 60 and 61;
(5) any one of SEQ ID NOs: 54 and 55;
(6) any one of SEQ ID NOs: 57 and 58;
(7) any one of SEQ ID NOs: 5, 29, 11, and 37 and any one of SEQ ID NOs: 3, 27, 4, 28, 12, and 38;
(8) any one of SEQ ID NOs: 5, 29, 11, and 37, any one of SEQ ID NOs: 3, 27, 4, 28, 12, and 38, and any one of SEQ ID NOs: 2, 26, 6, 32, 7, 33, 8, 34, 9, 35, 10 and 36;
(9) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, and any one of SEQ ID NOs: 60 and 61;
(10) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, any one of SEQ ID NOs: 54 and 55, and any one of SEQ ID NOs: 57 and 58; or
(11) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, any one of SEQ ID NOs: 54 and 55, any one of SEQ ID NOs: 57 and 58, and any one of SEQ ID NOs: 60 and 61.

Disclosed herein is a gene editing system comprising one or more guide RNA (e.g., sgRNA or crRNA) and an endonuclease or a nucleic acid encoding an endonuclease, in which the one or more guide RNA comprises a guide sequence or a sgRNA/crRNA sequence comprising:
(1) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37;
(2) any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38;
(3) any one or more of SEQ ID NOs: 2, 26, 6, 32, 7, 33, 8, 34, 9, 35, 10, and 36, and a sequence differing by no more than 5, 4, 3, 2, or 1 nucleotide(s) from any one or more of SEQ ID NOs: 2, 26, 6, 32, 7, 33, 8, 34, 9, 35, 10 and 36;
(4) any one of SEQ ID NOs: 60, 61, and a sequence differing by no more than 5, 4, 3, 2, or 1 nucleotide(s) from any one or more of SEQ ID NOs: 60 and 61;
(5) any one of SEQ ID NOs: 54, 55, and a sequence differing by no more than 5, 4, 3, 2, or 1 nucleotide(s) from any one or more of SEQ ID NOs: 54 and 55;
(6) any one of SEQ ID NOs: 57, 58, and a sequence differing by no more than 5, 4, 3, 2, or 1 nucleotide(s) from any one or more of SEQ ID NOs: 57 and 58;
(7) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37 and any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38;
(8) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, and any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36;
(9) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, and any one of SEQ ID NOs: 60, 61 and a sequence differing by no more than 5, 4, 3, 2, or 1 nucleotide(s) from any one or more of SEQ ID NOs: 60 and 61;
(10) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, any one of SEQ ID NOs: 54, 55, and a sequence differing by no more than 5, 4, 3, 2, or 1 nucleotide(s) from any one or more of SEQ ID NOs: 54 and 55, and any one of SEQ ID NOs: 57, 58, and a sequence differing by no more than 5, 4, 3, 2, or 1 nucleotide(s) from any one or more of SEQ ID NOs: 57 and 58;
(11) or any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, any one of SEQ ID NOs: 54, 55, and a sequence differing by no more than 5, 4, 3, 2, or 1 nucleotide(s) from any one or more of SEQ ID NOs: 54 and 55, any one of SEQ ID NOs: 57, 58, and a sequence differing by no more than 5, 4, 3, 2, or 1 nucleotide(s) from any one or more of SEQ ID NOs: 57 and 58, and any one of SEQ ID NOs: 60, 61 and a sequence differing by no more than 5, 4, 3, 2, or 1 nucleotide(s) from any one or more of SEQ ID NOs: 60 and 61.

Disclosed herein is a gene editing system comprising one or more sgRNA and an endonuclease or a nucleic acid encoding an endonuclease, in which the one or more sgRNA comprises a guide sequence that binds to a target sequence in a target gene. In some embodiments, the target sequence comprises:
(1) any one or more of SEQ ID NOs: 17, 23, 15, 16, and 24;
(2) any one or more of SEQ ID NOs: 14, 18, 19, 20, 21, and 22;
(3) SEQ ID NO: 62;
(4) SEQ ID NO: 56;
(5) SEQ ID NO: 59;
(6) any one or more of SEQ ID NOs: 17, 23, 15, 16, and 24 and any one or more of SEQ ID NOs: 14, 18, 19, 20, 21, and 22;
(7) any one or more of SEQ ID NOs: 17, 23, 15, 16, and 24, any one or more of SEQ ID NOs: 14, 18, 19, 20, 21, and 22, and SEQ ID NO: 62;
(8) any one or more of SEQ ID NOs: 17, 23, 15, 16, and 24, any one or more of SEQ ID NOs: 14, 18, 19, 20, 21, and 22, SEQ ID NO: 56, and SEQ ID NO: 59; or
(9) any one or more of SEQ ID NOs: 17, 23, 15, 16, and 24, any one or more of SEQ ID NOS: 14, 18, 19, 20, 21, and 22, SEQ ID NO: 62, SEQ ID NO: 56, and SEQ ID NO: 59.

In some embodiments, the target sequence comprises:
(1) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 17, 23, 15, 16, and 24;
(2) any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22;
(3) any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62;
(4) any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56;
(5) any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59;
(6) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24 and any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22;
(7) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, and any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62;
(8) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56, and any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59;
(9) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56, any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59, and any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62.

Suitable endonucleases for use in the disclosed gene editing systems may include endonucleases known in the art, including e.g., a Cas endonuclease. In some embodiments, the Cas endonuclease is a Cas9, Cas1212, Cas12a, Cas12b, Cas12c, Cas13a (C2c2), Cas13b, or MAD7.

In some embodiments, the endonuclease used in the CRISPR gene editing system is a MAD7 endonuclease; and the target sequence of the guide RNA of the CRISPR gene editing system comprises SEQ ID NO: 22 and/or the guide RNA comprises a sequence differing by no more than 5, 4, 3, 2, or 1 nucleotide(s) from SEQ ID NO: 10. In some embodiments, the endonuclease used in the CRISPR gene editing system is a MAD7 endonuclease; and the target sequence of the guide RNA of the CRISPR gene editing system comprises SEQ ID NO: 23 and/or the guide RNA comprises a sequence differing by no more than 5, 4, 3, 2, or 1 nucleotide(s) from SEQ ID NO: 11. In some embodiments, the endonuclease used in the CRISPR gene editing system is a MAD7 endonuclease; and, the target sequence of the guide RNA of the CRISPR gene editing system comprises SEQ ID NO: 24 and/or the guide RNA comprises a sequence differing by no more than 5, 4, 3, 2, or 1 nucleotide(s) from SEQ ID NO: 12.

In some embodiments, the endonuclease used in the CRISPR gene editing system is a Cas9 endonuclease; and the target sequence of the guide RNA of the CRISPR gene editing system comprises any one of SEQ ID NO: 14, 18, 19, 20, and 21, and/or the guide RNA comprises a sequence differing by no more than 5, 4, 3, 2, or 1 nucleotide(s) from any one of SEQ ID NOs: 2, 6, 7, 8, and 9. In some embodiments, the endonuclease used in the CRISPR gene editing system is a Cas9 endonuclease; and the target sequence of the guide RNA of the CRISPR gene editing system comprises SEQ ID NO: 17 and/or the guide RNA comprises a sequence differing by no more than 5, 4, 3, 2, or 1 nucleotide(s) from SEQ ID NO: 5. In some embodiments, the endonuclease used in the CRISPR gene editing system is a Cas9 endonuclease; and, the target sequence of the guide RNA of the CRISPR gene editing system comprises any one of SEQ ID NOs: 15 or 16 and/or the guide RNA comprises a sequence differing by no more than 5, 4, 3, 2, or 1 nucleotide(s) from SEQ ID NOs: 3 or 4. In some embodiments, the endonuclease used in the CRISPR gene editing system is a Cas9 endonuclease; and, the target sequence of the guide RNA of the CRISPR gene editing system comprises SEQ ID NOs: 62 and/or the guide RNA comprises a sequence differing by no more than 5, 4, 3, 2, or 1 nucleotide(s) from SEQ ID NO: 61. In some embodiments, the endonuclease used in the CRISPR gene editing system is a Cas9 endonuclease; and, the target sequence of the guide RNA of the CRISPR gene editing system comprises SEQ ID NOs: 56 and/or the guide RNA comprises a sequence differing by no more than 5, 4, 3, 2, or 1 nucleotide(s) from SEQ ID NO: 55. In some embodiments, the endonuclease used in the CRISPR gene editing system is a Cas9 endonuclease; and, the target sequence of the guide RNA of the CRISPR gene editing system comprises SEQ ID NOs: 59 and/or the guide RNA comprises a sequence differing by no more than 5, 4, 3, 2, or 1 nucleotide(s) from SEQ ID NO: 58.

In some embodiments, the guide RNA comprises SEQ ID NO: 5 (SOCS1), SEQ ID NO: 29 (SOCS1), SEQ ID NO: 11 (SOCS1), SEQ ID NO: 37 (SOCS1), SEQ ID NO: 27 (CISH), SEQ ID NO: 28 (CISH), SEQ ID NO: 12 (CISH), SEQ ID NO: 38 (CISH), SEQ ID NO: 2 (BIM), SEQ ID NO: 26 (BIM), SEQ ID NO: 6 (BIM), SEQ ID NO: 32 (BIM), SEQ ID NO: 7 (BIM), SEQ ID NO: 33 (BIM), SEQ ID NO: 8 (BIM), SEQ ID NO: 34 (BIM), SEQ ID NO: 9 (BIM), SEQ ID NO: 35 (BIM) SEQ ID NO: 10 (BIM), SEQ ID NO: 36 (BIM), SEQ ID NO: 61 (FAS), SEQ ID NO: 60 (FAS), SEQ ID NO: 55 (B2M), SEQ ID NO: 54 (B2M), SEQ ID NO: 58 (CIITA), or SEQ ID NO: 57 (CIITA).

In some embodiments, the guide RNA comprises a CRISPR RNA (crRNA) sequence, in which the crRNA sequence comprises a 2′-O-Methyl modification at each of the first three positions of the 5′ end, and three 2′-O-Methyl modifications at the 3′ end of the crRNA, but not the very last base of the crRNA (e.g., for a 56 nucleotide crRNA, at base 53, 54, 55 of the crRNA, but not at base 56; for a 60 nucleotide crRNA, at base 57, 58, and 59, but not at base 60), and a phosphorothioate linkage between each base in the first three positions of the 5′ end, and a phosphorothioate linkage between each base of the last 3 positions of the crRNA sequence at the 3′ end. In some embodiments, the last base is modified to facilitate use with MAD7. In some embodiments, the guide RNA binds to MAD7. In some embodiments, the methyl modifications at the 5′ end and the phosphorothioate linkages at the 3′ end are to increase the crRNA stability.

In some embodiments, the crRNA is about 56 bases long. In some embodiments, the crRNA sequence is 56 bases long, and comprises a 2′-O-Methyl modification at each of positions 53, 54, and 55 of the crRNA sequence on the 3′ end. In some embodiments, the crRNA sequence is 56 bases long, and comprises a 2′-O-Methyl modification at each of positions 53, 54, and 55 of the crRNA sequence on the 3′ end, and a 2′-O-Methyl modification at each of positions 1, 2, and 3 of the crRNA sequence at the 5′ end. In some embodiments, the crRNA sequence is 56 bases long, and comprises a 2′-O-Methyl modification at each of positions 53, 54, and 55 of the crRNA sequence on the 3′ end, and a 2′-O-Methyl modification at each of positions 1, 2, and 3 of the crRNA sequence at the 5′ end, and a phosphorothioate linkage between each base in the first three positions of the 5′ end, and a phosphorothioate linkage between each base of the last 3 positions of the crRNA sequence at the 3′ end. In some embodiments, the crRNA comprises SEQ ID NO: 36, SEQ ID NO: 37, or SEQ ID NO: 38, which include the above-described modification pattern. In some embodiments, the last nucleotide at the 3′ end of the crRNA is modified with a nucleotide that anchors the crRNA binding to MAD7. In some embodiments, the last nucleotide at the 3′ end of the crRNA is modified with a pseudoknot that anchors the crRNA binding to MAD7.

### B. Programmable Addition Via Site-Specific Target Elements (Paste)

In some embodiments, the gene editing system used to modify the cells herein (e.g., the iαβT cell, iPSC, iHSC, iCLP cell, or immature iT cell) is a Programmable Addition via Site-Specific Target Elements (PASTE) (see, e.g., Yarnall M T N, Ioannidi E I, Schmitt-Ulms C, Krajeski R N, Lim J, Villiger L, Zhou W, Jiang K, Garushyants S K, Roberts N, Zhang L, Vakulskas C A, Walker J A 2nd, Kadina A P, Zepeda A E, Holden K, Ma H, Xie J, Gao G, Foquet L, Bial G, Donnelly S K, Miyata Y, Radiloff D R, Henderson J M, Ujita A, Abudayyeh O O, Gootenberg J S. Drag-and-drop genome insertion of large sequences without double-strand DNA cleavage using CRISPR-directed integrases. Nat Biotechnol. 2023 April;41(4):500-512). The PASTE gene editing system comprises a guide RNA, e.g., an attachment site-containing guide RNA (atgRNA) that incorporates a prime-editing guide RNA (pegRNA) sequence, which both specifies the target site and encodes the desired genomic disruption, and an attachment site for serine integrases. PASTE further comprises a CRISPR-Cas9 nickase fused to a reverse transcriptase and a serine integrase. In some embodiments, the pegRNA targets SOCS1. In some embodiments, the pegRNA targets CISH. In some embodiments, the pegRNA targets BIM. In some embodiments, the pegRNA targets β2M. In some embodiments, the pegRNA targets CIITA. In some embodiments, the pegRNA targets FAS. In some embodiments, the PASTE gene is transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### C. Prime Editing

In some embodiments, the gene editing system used to modify the cells herein (e.g., the iαβT cell, iPSC, iHSC, iCLP cell, or immature iT cell) is a Prime Editing (see Anzalone A V, Randolph P B, Davis J R, Sousa A A, Koblan L W, Levy J M, Chen P J, Wilson C, Newby G A, Raguram A, Liu D R. Search-and-replace genome editing without double-strand breaks or donor DNA. Nature. 2019 December;576(7785):149-157). The Prime Editing gene editing system comprises a guide RNA, including e.g., a pegRNA, which both specifies the target site and encodes the desired genomic disruption, and a CRISPR-Cas9 endonuclease fused to a reverse transcriptase. In some embodiments, the pegRNA targets SOCS1. In some embodiments, the pegRNA targets CISH. In some embodiments, the pegRNA targets BIM. In some embodiments, the pegRNA targets β2M. In some embodiments, the pegRNA targets CIITA. In some embodiments, the pegRNA targets FAS. In some embodiments, the Prime Editing gene editing system is transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### D. Base Editing

In some embodiments, the gene editing system used to modify the cells herein (e.g., the iαβT cell, iPSC, iHSC, iCLP cell, or immature iT cell) is base editing (see, e.g., Gaudelli N M, Komor A C, Rees H A, Packer M S, Badran A H, Bryson D I, Liu D R. Programmable base editing of A·T to G·C in genomic DNA without DNA cleavage. Nature. 2017 Nov. 23;551(7681):464-471). Base editing comprises a base editor, including e.g., a cytosine base editor or an adenine base editor, and a guide sequence used to target the base editor to a specific nucleotide sequence. The base editor comprises a Cas9 nickase and a nucleoside deaminase, including e.g., a cytosine deaminase or an adenine deaminase. The Cas9 nickase cuts one strand of DNA at a specific location determined by the guide sequence, and the nucleoside deaminase removes an amino group from a specific type of nucleoside (e.g., cytosine or adenine). In some embodiments, the guide sequence targets SOCS1. In some embodiments, the guide sequence targets CISH. In some embodiments, the guide sequence targets BIM. In some embodiments, the sequence targets β2M. In some embodiments, the guide sequence targets CIITA. In some embodiments, the guide sequence targets FAS. In some embodiments, the base editing gene editing system is transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### E. Transcription Activator-Like Effector Nucleases (TALENS)

In some embodiments, the gene editing system used to modify the cells herein (e.g., the iαβT cell, iPSC, iHSC, iCLP cell, or immature iT cell) is a transcription activator-like effector nuclease (TALEN) (see, e.g., Christian M, Cermark T, Doyle E L, Schmidt C, Zhang F, Hummel A, Bogdanove A J, Voytas D F. Targeting DNA double-strand breaks with TAL effector nucleases. Genetics. 2010; 186:757-761. Further, see Miller J C, Tan S, Qiao G, Barlow K A, et al. A TALE nuclease architecture for efficient genome editing. Nat Biotechnol. 2011; 29:143-148. Also, see Mussolino C, Morbitzer R, Lütge F, Dannemann N, Lahaye T, Cathomen T. A novel TALE nuclease scaffold enables high genome editing activity in combination with low toxicity. Nucl Acids Res. 2011; 39:9283-9293). The TALEN gene editing system comprises the TALEN, which is a restriction endonuclease, e.g., Fokl, fused to a transcription activator-like effector (TALE) that is engineered to bind to a desired DNA sequence to promote DNA cleavage at a specific location. In some embodiments, two distinct TALENs each target distinct regions of SOCS1. In some embodiments, two distinct TALENs each target distinct regions of CISH. In some embodiments, two distinct TALENs each target distinct regions of BIM. In some embodiments, two distinct TALENs each target distinct regions of β2M. In some embodiments, two distinct TALENs each target distinct regions of CIITA. In some embodiments, two distinct TALENs each target distinct regions of FAS. In some embodiments, the TALENs are transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### F. Zinc-Finger Nucleases (ZENS)

In some embodiments, the gene editing system used to modify the cells herein (e.g., the iαβT cell, iPSC, iHSC, iCLP cell, or immature iT cell) is a zinc-finger nuclease (ZFN) (see, e.g., Kim Y-G, Cha J, Chandrasegaran S. Hybrid restriction enzymes: Zinc finger fusions to FokI cleavage domain. Proc Natl Acad Sci USA. 1996; 93:1156-1160). In some embodiments, the ZFN comprises an endonuclease, including, e.g., Fokl, fused to zinc finger, in which each zinc finger domain of a zinc finger targets a sequence. In some embodiments, two distinct ZFNs each target distinct regions of SOCS1. In some embodiments, two distinct ZFNs each target distinct regions of CISH. In some embodiments, two distinct ZFNs each target distinct regions of BIM. In some embodiments, two distinct ZFNs each target distinct regions of B2M. In some embodiments, two distinct ZFNs each target distinct regions of CIITA. In some embodiments, two distinct ZFNs each target distinct regions of FAS. In some embodiments, ZFNs are transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### G. Endonucleases

Described herein are restriction endonuclease for use in the methods described herein, and also as components of the gene editing systems. For example, homing endonucleases and meganucleases are provided. In some embodiments, meganucleases, restriction endonucleases, and/or homing endonucleases are engineered to confer a genomic disruption or inactivating nucleic acid mutation in any one or more of BIM, SOCS, and CISH; SOCS1 and CISH, and, optionally, BIM, B2M, CIITA, and/or FAS in the cells herein (e.g., the iαβT cell, iPSC, iHSC, iCLP cell, or immature iT cell). In some embodiments, one meganuclease targets SOCS1 and a second meganuclease targets CISH. In some embodiments, a third meganuclease targets BIM. In some embodiments, a fourth meganuclease targets β2M. In some embodiments, a fifth meganuclease targets CIITA. In some embodiments, a sixth meganuclease targets FAS. In some embodiments, one restriction endonuclease targets SOCS1 and a second restriction endonuclease targets CISH. In some embodiments, a third restriction endonuclease targets BIM. In some embodiments, a fourth restriction endonuclease targets β2M. In some embodiments, a fifth restriction endonuclease targets CIITA. In some embodiments, a sixth restriction endonuclease targets FAS. In some embodiments, one homing endonuclease targets SOCS1 and a second homing endonuclease targets CISH. In some embodiments, a third homing endonuclease targets BIM. In some embodiments, a fourth homing endonuclease targets β2M. In some embodiments, a fifth homing endonuclease targets CIITA. In some embodiments, a sixth homing endonuclease targets FAS. In some embodiments, the restriction endonuclease, homing endonucleases, and/or meganucleases are transfected into αβT-cells, e.g., iPSC-derived αβT-cells, and their precursors (e.g., iPSC (e.g., αβT-iPSC), or an intermediate cell differentiated from an iPSC (e.g., αβT-iHSC, αβT-iCLP, and immature T-iαβT cells)) that is used to generate a modified T-iαβT cell.

### H. Transfection

The present disclosure provides methods of modifying the cell used herein (e.g., the T-iαβT cell, the αβT-iPSC, T-iHSC, T-iCLP, or immature T-iαβT cell) by transfection, including e.g., electroporation (e.g., Nucleofection® (Lonza Cologne GmbH, Cologne Germany)), physical transfection (e.g., microinjection, particle bombardment, or phototransfection), lipid-mediated transfection, diethylaminoethyl (DEAE)-dextran transfection, calcium phosphate precipitation, cationic polymer transfection, or viral transfection.

To generate a genomic disruption or inactivating nucleic acid mutation, the cell can be transfected (e.g., nucleofected) with a gene editing system. In some embodiments, the gene editing system comprises an endonuclease, including, e.g., Cas9 or MAD7, and an sgRNA that targets SOCS1, CISH, BIM, B2M, CIITA, or FAS. In some embodiments, the cell (e.g., T-iαβT cell, αβT-iPSC, T-iHSC, T-iCLP, or immature T-iαβT cell) undergoes a sequential transfection of the gene editing system. For example, the cell (e.g., T-iαβT cell, αβT-iPSC, T-iHSC, T-iCLP, or immature T-iαβT cell) is transfected with a first endonuclease and an sgRNA that targets SOCS1; then, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets CISH; then, the cell (e.g., T-iαβT cell, αβT-iPSC, T-iHSC, T-iCLP, or immature T-iαβT cell) is transfected with an sgRNA that targets SOCS1 and, optionally, a second endonuclease or the first endonuclease.

In some embodiments, the cell (e.g., T-iαβT cell, αβT-iPSC, T-iHSC, T-iCLP, or immature T-iαβT cell) is transfected with a first endonuclease and an sgRNA that targets SOCS1; then, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets BIM and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets CISH; then, the cell (e.g., T-iαβT cell, αβT-iPSC, T-iHSC, T-iCLP, or immature T-iαβT cell) is transfected with an sgRNA that targets SOCS1 and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets BIM and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets SOCS1; then, the cell (e.g., T-iαβT cell, αβT-iPSC, T-iHSC, T-iCLP, or immature T-iαβT cell) is transfected with an sgRNA that targets BIM and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets CISH and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets CISH; then, the cell (e.g., T-iαβT cell, αβT-iPSC, T-iHSC, T-iCLP, or immature T-iαβT cell) is transfected with an sgRNA that targets BIM and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets SOCS1 and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets BIM; then, the cell (e.g., T-iαβT cell, αβT-iPSC, T-iHSC, T-iCLP, or immature T-iαβT cell) is transfected with an sgRNA that targets SOCS1 and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets CISH and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets BIM; then, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets SOCS1 and, optionally, a third endonuclease, the second endonuclease, and/or the first endonuclease.

In some embodiments, the cell (e.g., T-iαβT cell, αβT-iPSC, T-iHSC, T-iCLP, or immature T-iαβT cell) is transfected with a first endonuclease and an sgRNA that targets SOCS1; then, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets FAS and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets CISH; then, the cell (e.g., T-iαβT cell, αβT-iPSC, T-iHSC, T-iCLP, or immature T-iαβT cell) is transfected with an sgRNA that targets SOCS1 and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets FAS and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets SOCS1; then, the cell (e.g., T-iαβT cell, αβT-iPSC, T-iHSC, T-iCLP, or immature T-iαβT cell) is transfected with an sgRNA that targets FAS and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets CISH and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets CISH; then, the cell (e.g., T-iαβT cell, αβT-iPSC, T-iHSC, T-iCLP, or immature T-iαβT cell) is transfected with an sgRNA that targets FAS and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets SOCS1 and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets FAS; then, the cell (e.g., T-iαβT cell, αβT-iPSC, T-iHSC, T-iCLP, or immature T-iαβT cell) is transfected with an sgRNA that targets SOCS1 and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets CISH and, optionally, a third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with a first endonuclease and an sgRNA that targets FAS; then, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; then, the cell (e.g., T-iαβT cell, αβT-iPSC, T-iHSC, T-iCLP, or immature T-iαβT cell) is transfected with an sgRNA that targets SOCS1 and, optionally, a third endonuclease, the second endonuclease, and/or the first endonuclease.

In some embodiments, the cell (e.g., T-iαβT cell, αβT-iPSC, T-iHSC, T-iCLP, or immature T-iαβT cell) is transfected with a first endonuclease and an sgRNA that targets SOCS1; and, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; and, the cell (e.g., T-iαβT cell, αβT-iPSC, T-iHSC, T-iCLP, or immature T-iαβT cell) is transfected with an sgRNA that targets BIM and, optionally, a third endonuclease, the second endonuclease, or the first endonuclease; and, the cell (e.g., T-iαβT cell, αβT-iPSC, T-iHSC, T-iCLP, or immature T-iαβT cell) is transfected with an sgRNA that targets B2M and, optionally, a fourth endonuclease, the third endonuclease, the second endonuclease, or the first endonuclease; and, the cell (e.g., T-iαβT cell, αβT-iPSC, T-iHSC, T-iCLP, or immature T-iαβT cell) is transfected with an sgRNA that targets CIITA; and, optionally, a fifth endonuclease; the fourth endonuclease, the third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with the sgRNA that targets SOCS1, the sgRNA that targets CISH, the sgRNA that targets BIM, and the sgRNA that targets β2M, the sgRNA that targets CIITA in any order. In some embodiments, the cell (e.g., T-iαβT cell, αβT-iPSC, T-iHSC, T-iCLP, or immature T-iαβT cell) is transfected with the first endonuclease, the second endonuclease, the third endonuclease, the fourth endonuclease, the fifth endonuclease, or any combination thereof in any order.

In some embodiments, the cell (e.g., T-iαβT cell, αβT-iPSC, T-iHSC, T-iCLP, or immature T-iαβT cell) is transfected with a first endonuclease and an sgRNA that targets SOCS1; and, the cell (e.g., T-iαβT cell, αβT-iPSC, T-iHSC, T-iCLP, or immature T-iαβT cell) is transfected with an sgRNA that targets CISH and, optionally, a second endonuclease or the first endonuclease; and, the cell (e.g., T-iαβT cell, αβT-iPSC, T-iHSC, T-iCLP, or immature T-iαβT cell) is transfected with an sgRNA that targets BIM and, optionally, a third endonuclease, the second endonuclease, or the first endonuclease; and, the cell (e.g., T-iαβT cell, αβT-iPSC, T-iHSC, T-iCLP, or immature T-iαβT cell) is transfected with an sgRNA that targets B2M and, optionally, a fourth endonuclease, the third endonuclease, the second endonuclease, or the first endonuclease; and, the cell (e.g., T-iαβT cell, αβT-iPSC, T-iHSC, T-iCLP, or immature T-iαβT cell) is transfected with an sgRNA that targets CIITA; and, optionally, a fifth endonuclease, the fourth endonuclease, the third endonuclease, the second endonuclease, and/or the first endonuclease; and, the cell (e.g., T-iαβT cell, αβT-iPSC, T-iHSC, T-iCLP, or immature T-iαβT cell) is transfected with an sgRNA that targets FAS; and, optionally, a sixth endonuclease, the fifth endonuclease, the fourth endonuclease, the third endonuclease, the second endonuclease, and/or the first endonuclease. In some embodiments, the cell (e.g., T-iαβT cell, αβT-iPSC, T-iHSC, T-iCLP, or immature T-iαβT cell) is transfected with the sgRNA that targets SOCS1, the sgRNA that targets CISH, the sgRNA that targets BIM, the sgRNA that targets B2M, the sgRNA that targets CIITA, the sgRNA that targets B2M, and the sgRNA that targets FAS in any order. In some embodiments, the cell (e.g., T-iαβT cell, αβT-iPSC, T-iHSC, T-iCLP, or immature T-iαβT cell) is transfected with the first endonuclease, the second endonuclease, the third endonuclease, the fourth endonuclease, the fifth endonuclease, the sixth endonuclease or any combination thereof in any order.

### VI. Methods of Generating the Modified Cells

In some embodiments, methods of inhibiting expression of any one or more of a SOCS1 gene, a CISH gene and a BIM gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided.

In some embodiments, methods of inhibiting expression of a SOCS1 gene and a CISH gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided. In some embodiments, methods of inhibiting expression of a SOCS1 gene, a CISH gene, and a BIM gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided. In some embodiments, methods of inhibiting expression of a SOCS1 gene, a CISH gene, a BIM gene, and a FAS gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided. In some embodiments, methods of inhibiting expression of a SOCS1 gene, a CISH gene, a BIM gene, a B2M gene, and a CIITA gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided. In some embodiments, methods of inhibiting expression of a SOCS1 gene, a CISH gene, a BIM gene, a B2M gene, a CIITA gene, and a FAS gene in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided. In some embodiments, the method suppresses or eliminates expression of SOCS1 and CISH. In some embodiments, the method suppresses or eliminates expression of SOCS1, CISH, and BIM. In some embodiments, the method suppresses or eliminates expression of SOCS1, CISH, BIM, and FAS. In some embodiments, the method suppresses or eliminates expression of SOCS1, CISH, BIM, B2M, and CIITA. In some embodiments, the method suppresses or eliminates expression of SOCS1, CISH, BIM, B2M, CIITA, and FAS. In some instances, the expression of BIM_{EL} and BIM_{L} splice variants are reduced in the modified cell relative to that in an unmodified control (e.g., an unmodified T-iαβT-cell). In some embodiments, the expression of BIM_{S} splice variant is retained in the modified cell.

Described herein is a method of inhibiting expression of SOCS1 and/or CISH genes in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)) that is used to generate a modified T-iαβT cell. The method comprises using a gene editing system (e.g., gene editing system described in Section V) to modify the αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells (e.g., the T-iαβT cell, the αβT-iPSC, T-iHSC, T-iCLP, or immature T-iαβT cell) can be contacted with guide RNAs and an endonuclease or a nucleic acid encoding an endonuclease, wherein the contacting results in a genomic disruption in an endogenous SOCS1 gene that suppresses or eliminates expression of SOCS1 and/or a genomic disruption in an endogenous CISH gene that suppresses or eliminates expression of CISH. In some embodiments, the genomic disruption within SOCS1 and/or CISH is made by administering guide RNAs to the cells herein (e.g., the iαβT cell, iPSC, iHSC, iCLP cell, or immature iT cell). In some embodiments, the guide RNAs are sgRNAs. In some embodiments, the guide RNAs comprise any one or more of SEQ ID NOs: 5, 29, 11, 37 and/or any one or more of SEQ ID NOs: 3, 27, 4, 28, 12, or 38. In some embodiments, the endonuclease that is administered to the cell is a Cas endonuclease. In some embodiments, the Cas endonuclease that is administered to the cell is MAD7 endonuclease. The MAD7 endonuclease is an engineered class 2 type V-A CRISPR-Cas (Cas12a/Cpf1) system.

Described herein is a method of inhibiting expression of BIM gene in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)) that is used to generate a modified T-iαβT cell. The method comprises using a gene editing system (e.g., gene editing system described in Section V) to modify the αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells (e.g., the T-iαβT cell, the αβT-iPSC, T-iHSC, T-iCLP, or immature T-iαβT cell) can be contacted with guide RNAs and an endonuclease or a nucleic acid encoding an endonuclease, wherein the contacting results in a genomic disruption in an endogenous BIM gene that suppresses or eliminates expression of BIM, wherein the genomic disruption is in an exon 2C of an endogenous BIM gene, and the expression and/or function of BIM_{EL} and BIM_{L} splice variants are reduced in the modified cell relative to that in an unmodified control and the expression and/or function of BIM_{S} splice variant is retained in the modified cell. In some embodiments, the genomic disruption within BIM is made by administering guide RNAs to the cells herein (e.g., the iαβT cell, iPSC, iHSC, iCLP cell, or immature iT cell). In some embodiments, the guide RNAs are sgRNAs. In some embodiments, the guide RNAs comprise any one or more of SEQ ID NOs: 2, 26, 8, 34, 9, 35, 10 and 36. In some embodiments, the endonuclease that is administered to the cell is a Cas endonuclease. In some embodiments, the Cas endonuclease that is administered to the cell is MAD7 endonuclease. The MAD7 endonuclease is an engineered class 2 type V-A CRISPR-Cas (Cas12a/Cpf1) system.

Described herein is a method of inhibiting expression of a SOCS1, CISH, and BIM genes by causing a genomic disruption in the SOCS1, CISH, and BIM genes in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). The method comprises using a gene editing system (e.g., gene editing system described in Section V) to modify αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells herein (e.g., the iαβT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, and a genomic disruption in an endogenous BIM gene that suppresses or eliminates expression of BIM. In some embodiments, the expression and/or function of BIM_{EL} and BIM_{L} splice variants are reduced in the modified αβT-cell relative to that in an unmodified αβT-cell or relative to the αβT-cell (e.g., T-iαβT-cell) before modification. In some embodiments, the expression and/or function of BIM_{S} splice variant is retained in the modified αβT-cell.

Described herein is a method of inhibiting expression of a SOCS1, CISH, BIM, and FAS genes by causing a genomic disruption in the SOCS1, CISH, BIM, and FAS genes in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). The method comprises using a gene editing system (e.g., gene editing system described in Section V) to modify αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells herein (e.g., the iαβT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, and a genomic disruption in an endogenous FAS gene that suppresses or eliminates expression of FAS. In some embodiments, the expression and/or function of BIM_{EL} and BIM_{L} splice variants are reduced in the modified αβT-cell relative to that in an unmodified αβT-cell or relative to the αβT-cell (e.g., T-iαβT-cell) before modification. In some embodiments, the expression and/or function of BIM_{S} splice variant is retained in the modified αβT-cell.

Described herein is a method of inhibiting expression of a SOCS1, CISH, BIM, B2M, and CIITA genes by causing a genomic disruption in the SOCS1, CISH, BIM, B2M and CIITA genes in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). The method comprises using a gene editing system to modify the αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells herein (e.g., the iαβT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, a genomic disruption in an endogenous B2M gene that suppresses or eliminates expression of B2M, and a genomic disruption in an endogenous CIITA gene that suppresses or eliminates expression of CIITA. In some embodiments, the expression and/or function of BIM_{EL} and BIM_{L} splice variants are reduced in the modified αβT-cell relative to that in an unmodified αβT-cell or relative to the αβT-cell (e.g., T-iαβT-cell) before modification. In some embodiments, the expression and/or function of BIM_{S} splice variant is retained in the modified αβT-cell.

Described herein is a method of inhibiting expression of a SOCS1, CISH, BIM, B2M, CIITA, and FAS genes by causing a genomic disruption in the SOCS1, CISH, BIM, B2M, CIITA, and FAS genes in αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). The method comprises using a gene editing system to modify the αβT-cells, e.g., iPSC-derived αβT-cells, and their precursors (e.g., an iPSC (e.g., αβT-iPSC), or an intermediate cell differentiated from an iPSC (e.g., αβT-iHSC, αβT-iCLP, and immature T-iαβT cells)). For example, the cells herein (e.g., the iαβT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, a genomic disruption in an endogenous B2M gene that suppresses or eliminates expression of B2M, a genomic disruption in an endogenous CIITA gene that suppresses or eliminates expression of CIITA, and a genomic disruption in an endogenous FAS gene that suppresses or eliminates expression of FAS. In some embodiments, the expression and/or function of BIM_{EL} and BIM_{L} splice variants are reduced in the modified αβT-cell relative to that in an unmodified αβT-cell or relative to the αβT-cell (e.g., T-iαβT-cell) before modification. In some embodiments, the expression and/or function of BIM_{S} splice variant is retained in the modified αβT-cell.

In some embodiments, the genomic disruption and/or inactivating mutation of the SOCS1 and BIM genes; SOCS1, CISH, and BIM genes; SOCS1, CISH, BIM, and FAS genes; SOCS1, CISH, BIM, B2M, and CIITA genes; or, SOCS1, CISH, BIM, B2M, CIITA, and FAS genes is made by administering guide RNAs to a cell (e.g., a T-iαβT cell, the αβT-iPSC, T-iHSC, T-iCLP, or immature T-iαβT cell). In some embodiments, the guide RNAs are sgRNAs. In some embodiments, the guide RNAs that target the SOCS1 gene comprise any one or more of SEQ ID NOs: 5, 29, 11, 37; the guide RNAs that target the CISH gene comprise any one or more of SEQ ID NOs: 3, 27, 4, 28, 12, or 38; the guide RNAs that target the BIM gene comprise any one or more of SEQ ID NOs: 2, 26, 6, 32, 7, 33, 8, 34, 9, 35, 10, and 36; the guide RNAs that target the FAS gene comprise any one or more of SEQ ID NOs: 60 and 61; the guide RNAs that target the B2M gene comprise any one or more of SEQ ID NOs: 54 and 55; and the guide RNAs that target the CIITA gene comprise any one or more of SEQ ID NOs: 57 and 58. In some embodiments, a nuclease is administered to the cell (e.g., a T-iαβT cell, the αβT-iPSC, T-iHSC, T-iCLP, or immature T-iαβT cell) with the guide RNA. In some embodiments, the nuclease is an endonuclease. In some embodiments, the endonuclease that is administered to the cell is a Cas endonuclease. In some embodiments, the Cas endonuclease that is administered to a cell is a Cas9 endonuclease or a MAD7 endonuclease.

In some embodiments, the genomic disruption of the endogenous SOCS1 gene suppresses or eliminates expression of the SOCS1 gene in the modified αβT-cell (e.g., T-iαβT-cell) relative to an unmodified αβT-cell (e.g., an unmodified T-iαβT-cell) or relative to the αβT-cell (e.g., T-iαβT-cell) before modification. In some embodiments, the genomic disruption in the SOCS1 gene is in exon 2 of the SOCS1 gene. In some embodiments, the genomic disruption in the SOCS1 gene is an inactivating nucleic acid mutation. In some embodiments, the genomic disruption in the SOCS1 gene is an inactivating nucleic acid mutation in exon 2 of the SOCS1 gene. In some embodiments, a genomic disruption or inactivating nucleic acid mutation in the SOCS1 gene is an insertion, deletion, substitution, or any combination thereof. In some embodiments, the genomic disruption is within a target sequence in SOCS1 that comprises the nucleotide sequence of any one of SEQ ID NO: 17, SEQ ID NO: 23, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous CISH gene suppresses or eliminates expression of the CISH gene in the modified αβT-cell (e.g., T-iαβT-cell) relative to an unmodified αβT-cell (e.g., an unmodified T-iαβT-cell) or relative to the αβT-cell (e.g., T-iαβT-cell) before modification. In some embodiments, the genomic disruption in the CISH gene is in exon 3, exon 4, or a combination thereof of the CISH gene. In some embodiments, the genomic disruption in the CISH gene is an inactivating nucleic acid mutation. In some embodiments, the genomic disruption in the CISH gene is an inactivating nucleic acid mutation in exon 3, exon 4, or a combination thereof of the CISH gene. In some embodiments, a genomic disruption or inactivating nucleic acid mutation in the CISH gene is an insertion, deletion, substitution, or any combination thereof. In some embodiments, the genomic disruption is within a target sequence in CISH that comprises the nucleotide sequence of any one of SEQ ID NO: 15, SEQ ID NO: 16 or SEQ ID NO: 24 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous BIM gene in the modified αβT-cell reduces the expression and/or function of a splice variant of BIM, BIM_{EL}, relative to an unmodified αβT-cell generated from a αβT-iPSC (e.g., an unmodified T-iαβT-cell) or relative to the αβT-cell (e.g., T-iαβT-cell) before modification. In some embodiments, the genomic disruption of the endogenous BIM gene reduces the expression and/or function of a splice variant of BIM, BIM_{L}, relative to an unmodified αβT-cell generated from a αβT-iPSC (e.g., an unmodified T-iαβT-cell) or relative to the αβT-cell (e.g., T-iαβT-cell) before modification. In some embodiments, the genomic disruption of the endogenous BIM gene in the modified αβT-cell reduces the expression and/or function of a splice variants BIM_{EL} and BIM_{L} relative to an unmodified αβT-cell generated from a αβT-iPSC (e.g., an unmodified T-iαβT-cell) or relative to the αβT-cell (e.g., T-iαβT-cell) before modification. In some embodiments, the genomic disruption of the endogenous BIM gene in the modified αβT-cell retains the expression and/or function a splice variant of BIM, BIM_{S}, relative to an unmodified αβT-cell generated from a αβT-iPSC (e.g., an unmodified T-iαβT-cell) or relative to the αβT-cell (e.g., T-iαβT-cell) before modification. In some embodiments, the genomic disruption of the BIM gene is in exon 2C of an endogenous BIM gene. In some embodiments, the genomic disruption of the BIM gene is an inactivating nucleic acid mutation in exon 2C of an endogenous BIM gene. In some embodiments, the inactivating mutation is an insertion, deletion, or substitution. In some embodiments, the genomic disruption is within a target sequence in BIM that comprises the nucleotide sequence of any one of SEQ ID NO: 14, SEQ ID NO: 18, SEQ ID NO: 19, SEQ ID NO: 20, SEQ ID NO: 21, or SEQ ID NO: 22 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous FAS gene suppresses or eliminates expression of the FAS gene in the modified αβT-cell (e.g., T-iαβT-cell) relative to an unmodified αβT-cell (e.g., an unmodified T-iαβT-cell) or relative to the αβT-cell (e.g., T-iαβT-cell) before modification. In some embodiments, the genomic disruption in the FAS gene is in exon 1 of the FAS gene. In some embodiments, the genomic disruption in the FAS gene is an inactivating nucleic acid mutation. In some embodiments, the genomic disruption in the FAS gene is an inactivating nucleic acid mutation in exon 1 of the FAS gene. In some embodiments, a genomic disruption or inactivating nucleic acid mutation in the FAS gene is an insertion, deletion, substitution, or any combination thereof. In some embodiments, the genomic disruption is within a target sequence in FAS that comprises the nucleotide sequence of SEQ ID NO: 62 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous β2M gene suppresses or eliminates expression of the β2M gene in the modified αβT-cell (e.g., T-iαβT-cell) relative to an unmodified αβT-cell (e.g., an unmodified T-iαβT-cell) or relative to the αβT-cell (e.g., T-iαβT-cell) before modification. In some embodiments, the genomic disruption in the B2M gene is in exon 2 of the B2M gene. In some embodiments, the genomic disruption in the B2M gene is an inactivating nucleic acid mutation. In some embodiments, the genomic disruption in the B2M gene is an inactivating nucleic acid mutation in exon 2 of the B2M gene. In some embodiments, a genomic disruption or inactivating nucleic acid mutation in the B2M gene is an insertion, deletion, substitution, or any combination thereof. In some embodiments, the genomic disruption is within a target sequence in B2M that comprises the nucleotide sequence of SEQ ID NO: 56 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous CIITA gene suppresses or eliminates expression of the CIITA gene in the modified αβT-cell (e.g., T-iαβT-cell) relative to an unmodified αβT-cell (e.g., an unmodified T-iαβT-cell) or relative to the αβT-cell (e.g., T-iαβT-cell) before modification. In some embodiments, the genomic disruption in the CIITA gene is in exon 3 of the CIITA gene. In some embodiments, the genomic disruption in the CIITA gene is an inactivating nucleic acid mutation. In some embodiments, the genomic disruption in the CIITA gene is an inactivating nucleic acid mutation in exon 3 of the CIITA gene. In some embodiments, a genomic disruption or inactivating nucleic acid mutation in the CIITA gene is an insertion, deletion, substitution, or any combination thereof. In some embodiments, the genomic disruption is within a target sequence in CIITA that comprises the nucleotide sequence of SEQ ID NO: 59 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, a genomic disruption or inactivating nucleic acid mutation in the SOCS1, CISH, BIM, B2M, CIITA, and/or FAS gene is an insertion, deletion, substitution, or any combination thereof.

In some embodiments, the method comprises contacting the cell with guide RNAs and an endonuclease or a nucleic acid encoding an endonuclease, wherein the contacting results in a genomic disruption in an endogenous SOCS1 gene that suppresses or eliminates expression of SOCS1 and a genomic disruption in an endogenous CISH gene that suppresses or eliminates expression of CISH. If BIM is also being disrupted, then a guide RNA is used that results in inhibiting expression of an endogenous BIM gene. If B2M is also being disrupted, then a guide RNA is used that results in inhibiting expression of an endogenous B2M gene. If CIITA is also being disrupted, then a guide RNA is used that results in inhibiting expression of an endogenous CIITA gene. If FAS is also being disrupted, then a guide RNA is used that results in inhibiting expression of an endogenous FAS gene.

Further information on the guide RNA and endonuclease could be found in other sections of the present disclosure, e.g., Section V: Gene editing systems.

Further information on the cells used in the methods of inhibiting could be found in other sections of the present disclosure, e.g., Section II: Modified cells and incorporated components and Section III: Modified cells comprising CAR and/or TCRαβ, e.g., anti-KRAS G12V TCR.

In some embodiments, methods of introducing any one or more exogenous polynucleotides encoding a signal converter in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided.

In some embodiments, methods of introducing any one or more of the exogenous polynucleotides encoding anti-KRAS G12V TCR, B2M-HLA-E, and signal converter in αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are provided.

In some embodiments, introducing any one or more exogenous polynucleotides to αβT-cells, e.g., iPSC-derived αβT-cells (and their precursors (e.g., an iPSC (e.g., αβT-iPSC), or an intermediate cell differentiated from an iPSC (e.g., αβT-iHSC, αβT-iCLP, and immature T-iαβT cells))) are achieved by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair.

In some embodiments, the present disclosure provides the modified cells generated from the method of inhibiting expression and/or method of introducing exogenous polynucleotides described herein.

The present disclosure provides a method of generating the modified αβT cells, comprising (i) generating the modified iPSC cells described herein (e.g., Section II and III), and (ii) differentiating the modified iPSC cells into the modified αβT cells.

### VII. Pharmaceutical Compositions

Pharmaceutical compositions comprising any of the modified cells or populations of cells (e.g., modified αβT) are encompassed. In some embodiments, pharmaceutical composition comprises the modified cell or population of cells (e.g., modified αβT) and a pharmaceutically acceptable carrier.

### VIII. Methods of Treatment

Described herein are methods of treating diseases and disorders using the modified cells, population of cells, or pharmaceutical compositions described herein.

In some embodiments, the present disclosure provides a method of killing cancer cells which are KRAS G12V positive, comprising administering to a subject in need thereof a therapeutically effective amount of the modified αβT cell described herein or the pharmaceutical composition described herein.

In some embodiments, the present disclosure also provides a method of treating colorectal cancer with KRAS G12V, comprising administering to a subject in need thereof a therapeutically effective amount of the modified αβT cell described herein or the pharmaceutical composition described herein.

In some embodiments, the present disclosure also provides the modified αβT cell described herein or the pharmaceutical composition described herein for use in therapy.

In some embodiments, the present disclosure also provides the modified αβT described herein or the pharmaceutical composition described herein for use in treatment of colorectal cancer with KRAS G12V.

In some embodiments, the present disclosure also provides use of the modified cell, (e.g., the modified αβT cell, the modified iPSC cells described herein) or the pharmaceutical composition described herein in the manufacturing of a medicament for treatment of colorectal cancer with KRAS G12V.

In some embodiments, the present disclosure provides a method of killing B cells which are CD19 positive and have abnormal B cell function, comprising administering to a subject in need thereof a therapeutically effective amount of the modified αβT cell described herein or the pharmaceutical composition described herein.

In some embodiments, the present disclosure provides a method of killing B cells which are CD20 positive and have abnormal B cell function, comprising administering to a subject in need thereof a therapeutically effective amount of the modified αβT cell described herein or the pharmaceutical composition described herein.

In some embodiments, the present disclosure provides a method of killing B cells which are CD19 positive and CD20 positive and have abnormal B cell function, comprising administering to a subject in need thereof a therapeutically effective amount of the modified αβT cell described herein or the pharmaceutical composition described herein.

In some embodiments, the B cells are the CD19 positive B cells in B-cell Acute Lymphoblastic Leukemia (B-ALL) patients.

In some embodiments, the present disclosure provides a method of treating B-ALL, comprising administering to a subject in need thereof a therapeutically effective amount of the modified αβT cell described herein or the pharmaceutical composition described herein.

In some embodiments, the present disclosure provides the modified αβT cell described herein or the pharmaceutical composition described herein for use in therapy.

In some embodiments, the present disclosure provides the modified αβT cell described herein or the pharmaceutical composition described herein for use in treatment of B-ALL.

In some embodiments, the present disclosure provides use of the modified cell (e.g., the modified αβT cell, the modified iPSC cells described herein) described herein or the pharmaceutical composition described herein in the manufacturing of a medicament for treatment of B-ALL.

The abbreviation, “e.g.,” is derived from the Latin exempli gratia, and is used herein to indicate a non-limiting example. Thus, the abbreviation “e.g.,” is synonymous with the term “for example.” The abbreviation, “i.e.,” is derived from the Latin id est, and is used herein to indicate a non-limiting rewording or clarification. Thus, the abbreviation “i.e.,” is synonymous with the term “that is.”

Section headings, materials, methods, and examples are illustrative only and not intended to be limiting.

### Examples

Example 1. Generation of BIM^{-/-}CISH^{-/-}SOCS1^{-/-} Triple Knockout (TKO) and BIM^{-/-}CISH^{-/-}SOCS1^{-/-}FAS^{-/-} Quadruple Knockout (QKO) KRAS G12V TCR TiPSC-αβT Cells

### Nucleofection

The T-iPSC cell line (reprogrammed from human primary αβT cells) was used to generate KRAS G12V TCR expressing iPSCs (comprising KRAS G12V TCR alpha (SEQ ID NO: 79) and KRAS G12V TCR beta (SEQ ID NO: 80), refer to WO2019112941A1 for detail). KRAS G12V TCR in TCRbeta-p2A-TCRalpha was knock-in to the TRAC locus of T-iPSC, to improve the differentiation efficiency from iPSC to αβT without functional TRAC. Briefly, Cas9 and sgRNA targeted to the TRAC locus were mixed to prepare the RNP complex (refer to Eyquem, Justin, et al. “Targeting a CAR to the TRAC locus with CRISPR/Cas9 enhances tumour rejection” Nature 543.7643 (2017): 113 for details). The RNP complex was mixed with the KRAS G12V TCR donor plasmid and delivered to T-iPSCs by 4D nucleofection kit (V4XC-9064, Lonza). The KRAS G12V TCR knock-in T-iPSC cells were seeded into a vitronectin (VTN) (Thermo Fisher Scientific #A14700) coated 96 well plate for single colony formation. The single cell clone was isolated and identified by detecting the TCR knock-in TRAC locus using PCR with specific primer sets. KRAS G12V TCR knock-in αβT-derived iPSC cells were further differentiated to KRAS G12V TCR T-iαβT (CD8αβ⁺) cells.

To generate target gene knockout in KRAS G12V TCR T-iαβT (CD8αβ⁺) cells from an KRAS G12V TCR knock-in αβT-derived iPSC (iPSC-derived αβT, wherein the iPSC is generated from a primary αβT cell), 5×10⁶ of KRAS G12V TCR T-iαβT cells were sequentially nucleofected with 200 μmol of Alt-R™ S.p. HiFi Cas9 Nuclease V3 (Integrated DNA Technologies (Coralville, IA); Cat: 1081060) and 1,500 μmol of synthetic single guide RNA (sgRNAs) by Lonza P3 Primary Cell 4D-Nucleofector® X Kit L (Lonza (Basel, Switzerland), Cat: V4XP-3024) and Lonza 4D Nucleofector® Core and X Unit (Lonza, Cat: AAF-1003X) per Nucleocuvette vessel. To create a double knockout (DKO), triple knockout (TKO) or quadruple knockout (QKO) KRAS G12V TCR T-iαβT cell, KRAS G12V TCR T-iαβT cells were sequentially nucleofected to ensure the knockout efficiency of each target gene. Nucleofected T-iαβT cells were cultured with culture medium (AIM V™ Medium (Gibco; Cat: 12055091) supplemented with AA2P (250 ng/mL; Cayman, Cat: 16457), EliteMu™ human platelet lysate (HPL) (5%; EliteCell, Cat: EPI-500)) supplemented with recombinant human IL-7 (10 ng/mL; PeproTech (Cranbury, NJ), Cat: 200-07), and recombinant human IL-15 (10 ng/mL; PeproTech, Cat: 200-15) at cell density of 1×10⁶ cells/mL every 2 days prior to next round of nucleofection.

TABLE 1
Sequences of KRAS G12V TCR

| | | |
|---|---|---|
| SEQ ID NO: 79 | KRAS G12V TCR | MHLSSLLKVVTASLWLGPGIAQKITQTQPGMFVQEKEAVTLD |
| | alpha | CTYDTSDPSYGLFWYKQPSSGEMIFLIYQGSYDQQNATEGRY |
| | | SLNFQKARKSANLVISASQLGDSAMYFCAMRGASQGGSEKLV |
| | | FGKGTKLTVNPDIQNPEPAVYQLKDPRSQDSTLCLFTDFDSQIN |
| | | VPKTMESGTFITDKCVLDMKAMDSKSNGAIAWSNQTSFTCQ |
| | | DIFKETNATYPSSDVPCDATLTEKSFETDMNLNFQNLSVMGLR |
| | | ILLLKVAGFNLLMTLRLWSS |
| SEQ ID NO: 80 | KRAS G12V TCR | MASRLLCWVLLCLLGAGPVKAGVTQTPRYLIKTRGQQVTLS |
| | beta | CSPISGHRSVSWYQQTPGQGLQFLFEYFSETQRNKGNFPGRFS |
| | | GRQFSNSRSEMNVSTLELGDSALYLCASSLTSGGFDEQFFGPG |
| | | TRLTVLEDLRNVTPPKVSLFEPSKAEIANKQKATLVCLARGFF |
| | | PDHVELSWWVNGKEVHSGVCTDPQAYKESNYSYCLSSRLRV |
| | | SATFWHNPRNHFRCQVQFHGLSEEDKWPEGSPKPVTQNISAE |
| | | AWGRADCGITSASYQQGVLSATILYEILLGKATLYAVLVSTLVV |
| | | MAMVKRKNS |
| SEQ ID NO: 82 | KRAS G12V TCR | AQKVTQAQTEISVVEKEDVTLDCVYETRDTTYYLFWYKQPPS |
| | alpha | GELVFLIRRNSFDEQNEISGRYSWNFQKSTSSFNFTITASQVVD |
| | | SAVYFCALSDPRYSGGGADGLTFGKGTHLIIQPYIQNPDPAVY |
| | | QLRDSKSSDKSVCLFTDFDSQTNVSQSKDSDVYITDKTVLDM |
| | | RSMDFKSNSAVAWSNKSDFACANAFNNSIIPEDTFFPSPESSCD |
| | | VKLVEKSFETDTNLNFQNLSVIGFRILLLKVAGFNLLMTLRLW |
| | | SS |
| SEQ ID NO: 83 | KRAS G12V TCR | DSGVTQTPKHLITATGQRVTLRCSPRSGDLSVYWYQQSLDQG |
| | beta | LQFLIQYYNGEERAKGNILERFSAQQFPDLHSELNLSSLELGD |
| | | SALYFCASSPRGGDSILDTQYFGPGTRLTVLEDLKNVFPPEVAV |
| | | FEPSEAEISHTQKATLVCLATGFYPDHVELSWWVNGKEVHSG |
| | | VSTDPQPLKEQPALNDSRYCLSSRLRVSATFWQNPRNHFRCQ |
| | | VQFYGLSENDEWTQDRAKPVTQIVSAEAWGRADCGFTSESY |
| | | QQGVLSATILYEILLGKATLYAVLVSALVLMAMVKRKDSRG |

Nucleofection with Cas9 Nuclease and sgRNAs in KRAS G12V TCR T-iαβT Cells

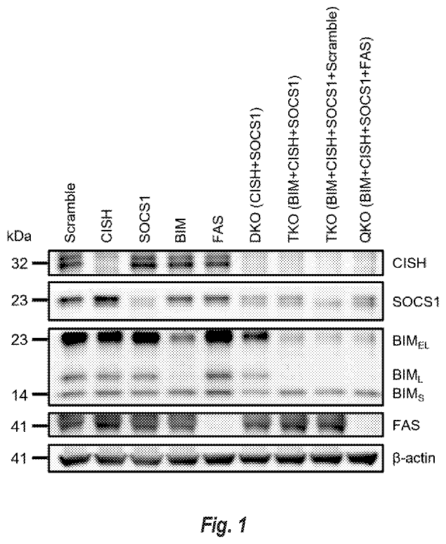

To generate the single, double, triple or quadruple target gene knockout KRAS G12V TCR T-iαβT cells, 5×10⁶ KRAS G12V TCR T-iαβT cells were nucleofected with 200 μmol of Alt-R™ S.p. HiFi Cas9 Nuclease V3 (Integrated DNA Technologies; Cat: 1081060) and 1,500 pmol of synthetic single guide RNA (sgRNAs) as listed in Table 2 by Lonza P3 Primary Cell 4D-Nucleofector® X Kit L (Lonza, Cat: V4XP-3024) and Lonza 4D Nucleofector® Core and X Unit (Lonza, Cat: AAF-1003X) per Nucleocuvette vessel. Each target gene was knocked out by one synthetic sgRNA except CISH, which was knocked out by simultaneously using two sgRNAs (CISH #3 and #4). To ensure the knockout efficiency of each target gene, KRAS G12V TCR T-iαβT cells were sequentially nucleofected. Since the cytotoxic efficacy and survival of T-iαβT cells would be affected by nucleofection, all groups were nucleofected for a total of three or four rounds. For the single knockout group, KRAS G12V TCR T-iαβT cells were nucleofected with target gene sgRNA(s) in a first round of nucleofection followed with scramble sgRNA for second and third round of nucleofection. For DKO/TKO/QKO, the KRAS G12V TCR T-iαβT cells were sequentially nucleofected in the order as shown in the parentheses of FIG. 1 and reproduced here: DKO (CISH+SOCS1) has the KRAS G12V TCR T-iαβT cell nucleofected with sgRNAs targeting CISH, and then, with sgRNA targeting SOCS1; TKO (BIM+CISH+SOCS1) has the KRAS G12V TCR T-iαβT cell nucleofected with sgRNA targeting BIM, then sgRNAs targeting CISH, and finally, with sgRNA targeting SOCS1; QKO (BIM+CISH+SOCS1+FAS) has the KRAS G12V TCR T-iαβT cell nucleofected with sgRNA targeting BIM, then sgRNAs targeting CISH and SOCS1, and finally, with sgRNA targeting FAS. Nucleofected T-iαβT cells were cultured with culture medium (AIM V™ Medium (Gibco; Cat: 12055091) supplemented with AA2P (250 ng/mL; Cayman, Cat: 16457), EliteMu™ human platelet lysate (HPL) (5%; EliteCell, Cat: EPI-500)) supplemented with recombinant human IL-7 (10 ng/mL; PeproTech (Cranbury, NJ), Cat: 200-07), and recombinant human IL-15 (10 ng/mL; PeproTech, Cat: 200-15) at cell density of 1×10⁶ cells/mL every 2 days prior to next round of nucleofection.

TABLE 2
sgRNA sequence of candidate genes used for nucleofection with Cas9 nuclease

| | | | Targeting/ | Target | |
|---|---|---|---|---|---|
| | Name | | guide | sequence | |
| Target | of | | sequence in | in target | Target |
| Gene | sgRNA | Synthetic sgRNA sequence | sgRNA | gene | exon |
| / | Scramble | mG*mC*mA*rCrUrCrArCrArU | GCACTCACA | TGATGTAG | / |
| | sgRNA | rCrGrCrUrArCrArUrCrArGrUr | TCGCTACAT | CGATGTGA | |
| | (Scramble | UrUrUrArGrArGrCrUrArGrAr | CA | GTGC | |
| | #2) | ArArUrArGrCrArArGrUrUrAr | (SEQ ID NO: | (SEQ ID NO: | |
| | | ArArArUrArArGrGrCrUrArGr | 1) | 13) | |
| | | UrCrCrGrUrUrArUrCrArArCrU | | | |
| | | rUrGrArArArArArGrUrGrGrCr | | | |
| | | ArCrCrGrArGrUrCrGrGrUrGrC | | | |
| | | mU*mU*mU*rU (SEQ ID NO: | | | |
| | | 25) | | | |
| BIM | BIM | mU*mA*mC*rCrCrArUrUrGrC | TACCCATTG | CTATCTCA | Exon |
| (BCL2 | sgRNA | rArCrUrGrArGrArUrArGrGrUr | CACTGAGAT | GTGCAATG | 2C |
| L11) | (BIM | UrUrUrArGrArGrCrUrArGrAr | AG | GGTA | |
| | # 5) | ArArUrArGrCrArArGrUrUrAr | (SEQ ID NO: | (SEQ ID NO: | |
| | | ArArArUrArArGrGrCrUrArGr | 2) | 14) | |
| | | UrCrCrGrUrUrArUrCrArArCrU | | | |
| | | rUrGrArArArArArGrUrGrGrCr | | | |
| | | ArCrCrGrArGrUrCrGrGrUrGrC | | | |
| | | mU*mU*mU*rU (SEQ ID NO: | | | |
| | | 26) | | | |
| CIS | CISH | mA*mG*mG*rCrCrArCrArUr | AGGCCACAT | TGTGCAGC | Exon |
| (CISH) | sgRN | ArGrUrGrCrUrGrCrArCrArGr | AGTGCTGCA | ACTATGTG | 4 |
| | A | UrUrUrUrArGrArGrCrUrArGr | CA | GCCT | |
| | (CISH | ArArArUrArGrCrArArGrUrUr | (SEQ ID NO: | (SEQ ID NO: | |
| | #3) | ArArArArUrArArGrGrCrUrAr | 3) | 15) | |
| | | GrUrCrCrGrUrUrArUrCrArArC | | | |
| | | rUrUrGrArArArArArGrUrGrGr | | | |
| | | CrArCrCrGrArGrUrCrGrGrUrG | | | |
| | | rCmU*mU*mU*rU (SEQ ID | | | |
| | | NO: 27) | | | |
| | CISH | mU*mG*mU*rArCrArGrCrAr | TGTACAGCA | CCACCAGC | Exon |
| | sgRNA | GrUrGrGrCrUrGrGrUrGrGrGr | GTGGCTGGT | CACTGCTG | 4 |
| | (CISH | UrUrUrUrArGrArGrCrUrArGr | GG | TACA | |
| | #4) | ArArArUrArGrCrArArGrUrUr | (SEQ ID NO: | (SEQ ID NO: | |
| | | ArArArArUrArArGrGrCrUrAr | 4) | 16) | |
| | | GrUrCrCrGrUrUrArUrCrArArC | | | |
| | | rUrUrGrArArArArArGrUrGrGr | | | |
| | | CrArCrCrGrArGrUrCrGrGrUrG | | | |
| | | rCmU*mU*mU*rU (SEQ ID | | | |
| | | NO: 28) | | | |
| SOCS1 | SOCS1 | mA*mG*mU*rArGrArArUrCr | AGTAGAATC | GGACGCCT | Exon |
| | sgRNA | CrGrCrArGrGrCrGrUrCrCrGrU | CGCAGGCGT | GCGGATTC | 2 |
| | #2 | rUrUrUrArGrArGrCrUrArGrAr | CC | TACT | |
| | | ArArUrArGrCrArArGrUrUrAr | (SEQ ID NO: | (SEQ ID NO: | |
| | | ArArArUrArArGrGrCrUrArGr | 5) | 17) | |
| | | UrCrCrGrUrUrArUrCrArArCrU | | | |
| | | rUrGrArArArArArGrUrGrGrCr | | | |
| | | ArCrCrGrArGrUrCrGrGrUrGrC | | | |
| | | mU*mU*mU*rU (SEQ ID NO: | | | |
| | | 29) | | | |
| FAS | FAS | mG*mA*mU*rUrGrCrUrCrAr | GATTGCTCA | AGCATGGT | Exon |
| | sgRNA | ArCrArArCrCrArUrGrCrUrGrU | ACAACCATG | TGTTGAGC | 1 |
| | (FAS # | rUrUrUrArGrArGrCrUrArGrAr | CT | AATC | |
| | 1) | ArArUrArGrCrArArGrUrUrAr | (SEQ ID NO: | (SEQ ID NO: | |
| | | ArArArUrArArGrGrCrUrArGr | 61) | 62) | |
| | | UrCrCrGrUrUrArUrCrArArCrU | | | |
| | | rUrGr ArArArArArGrUrGrGrCr | | | |
| | | ArCrCrGrArGrUrCrGrGrUrGrC | | | |
| | | mU*mU*mU*rU (SEQ ID NO: | | | |
| | | 60) | | | |

“*” means the phosphorothioate linkage between each base, and “m” mean 2′-O-Methyl modification.

### Western Blotting

To validate the knockout efficiency of target genes in KRAS G12V TCR T-iαβT cells post nucleofection, Western blotting was performed to examine the protein expression level of various genes. Briefly, T-iαβT cells were harvested, and cell pellets were lysed in radioimmunoprecipitation (RIPA) buffer (Millipore (Burlington, MA), Cat: 20-188) containing protease inhibitor cocktail (TargetMol (Boston, MA), Cat: C0001). The mixture was pipetted gently and put on ice for 30 minutes and then centrifuged at 12,000×g for 15 minutes at 4 °C. The supernatants were transferred to a new 1.6 mL microcentrifuge tube. The protein concentration of the cell lysate was determined with Protein Assay Dye Reagent (Bio-Rad (Hercules, CA), Cat: 5000006) and Multiskan SkyHigh Microplate Spectrophotometer (ThermoFisher Scientific). A total of 20 mg of protein in each lane was separated with mPAGE 4-20% Bis-Tris Precast Gel (Millipore, Cat: MP42G12). The separated proteins were subsequently transferred to Power Blotter Select Transfer polyvinylidene fluoride (PVDF) membrane (Invitrogen (Waltham, MA), Cat: PB5340). The gel and the PVDF membrane were carefully removed from the stack. The blot was rinsed with 1×TBST (Tris buffered saline and polysorbate 20). The transferred PVDF membrane was blocked with 5% milk in TBST at room temperature for 1 hour. The blot was rinsed three times for 10 minutes with TBST and then incubated with a BIM-targeting antibody (1:1,000; Cell Signaling Technology (Danvers, MA), Cat: 2933S), a CISH-targeting antibody (1:1,000; Cell Signaling Technology, Cat: 8731S), a SOCS1-targeting antibody (1:1,000; Cell Signaling Technology, Cat: 68631S) and a FAS-targeting antibody (1:1,000; Proteintech, Cat: 13098-1-AP) in 1% milk/TBST overnight at 4 °C. The blot was rinsed three times for 10 minutes with TBST and incubated with Peroxidase conjugated goat anti-rabbit IgG antibody (1:5,000; Sigma-Aldrich (St. Louis, MO), Cat: AP132P) in 1% milk/TBST at room temperature for 1 hour. The blot was rinsed three times for 10 minutes with TBST. The SuperSignal West Pico PLUS Chemiluminescent Substrate (ThermoFisher Scientific, Cat: 34580) was applied to the blot according to the manufacturer's recommendation. The chemiluminescent signals were captured using the iBright FL1500 Imaging System (ThermoFisher Scientific). Beta-actin (1:4,000; Santa Cruz Biotechnology (Dallas, TX), Cat: sc-47778 HRP) was used as an internal reference. Western blotting result confirmed the efficient knockout of target genes by nucleofection with Cas9 nuclease and synthetic sgRNAs (FIG. 1).

As shown in FIG. 1, Western blotting results showed that CISH sgRNAs (CISH #3 and #4) and SOCS1 sgRNA could efficiently knock out their protein expression. Human BIM gene, also called BCL2L11 (Gene ID: 10018), is transcribed as three splice variants, BIM_{EL} (NM_138621.5; NP_619527.1, containing coding exons E1, E2A, E2B, E2C, E4 and E5), BIM_{L} (NM_006538.5; NP_006529.1, containing coding exons E1, E2A, E2C, E4 and E5) and BIM_{S} (NM_001204106.1; NP_001191035.1, containing coding exons E1, E2A, E4 and E5). The expression level of BIM_{EL} was higher than BIM_{L} and BIM_{S} splice variants in KRAS G12V TCR T-iαβT cells. For BIM knockout in KRAS G12V TCR T-iαβT cells, nucleofection was performed with BIM sgRNA #5, which targets BIM exon E2C, hence the knockout of BIM_{EL} and BIM_{L} splice variants while preserving the BIM_{S} splice variant in KRAS G12V TCR T-iαβT cells. Western blotting results showed BIM_{L} was a complete knockout, and BIM_{EL} was a substantial and efficient knockout with trace amounts shown in Western blotting. For example, the percentages of BIM_{EL} in BIM single KO, DKO, TKO and QKO groups range from about 10.4% to about 45.7% relative to the scramble group. Overall, BIM sgRNA #5 could efficiently knock out the BIM_{EL} and BIM_{L} splice variants, while preserving the BIM_{S} splice variant.

### Indel Percentage by ICE Analysis

2×10⁵ of genome-edited T-iαβT cells were harvested, and the genomic DNA was extracted with QuickExtract™ DNA Extraction Solution 1.0 (LGC, Teddington, England, Cat: SS000035-D2). Target gene-specific DNA fragment was amplified by KAPA HiFi HotStart PCR Kit ((Roche, Basel, Switzerland), Cat: KR0369) with specific primer pairs as listed in Table 3 and following amplification program:
Step 1, 95 °C. for 3 minutes
Step 2, 98 °C. for 20 seconds
Step 3, Melting temperature (Tm °C.) for 15 seconds
Step 4, 72 °C. for 60 seconds
Repeat Step 2-4 for 30 cycles
Step 5, 72 °C. for 60 seconds

The PCR products were sequenced by MISSION BIOTECH (Taipei City, Taiwan). The primer sequences used for sequencing were the same as the PCR primer sequences. The Indel percentages were analyzed by Synthego (Redwood City, CA) Inference of CRISPR Edits (ICE) Analysis. Briefly, the sequencing results of target gene knockout group and the scramble control were imported into the Synthego ICE analysis tool. The indel percentage and knockout score were acquired after the Synthego ICE analysis (Table 4). The results of the indel percentage showed an efficient knockout of each target gene by sequential nucleofection with Cas9 nuclease and sgRNAs.

TABLE 3
Primer sequence of candidate genes for ICE analysis

| Primer | Sequence (5′>3′) | Tm (°C) |
|---|---|---|
| BIM forward | TTTGCTACCAGATCCCCGCT (SEQ ID NO: 39) | 65 °C |
| BIM reverse | CCAAACCCTCTGAGCAATAAGCA (SEQ ID NO: 40) | |
| CISH forward | GCACTGCCTTGACTTGCACT (SEQ ID NO: 41) | 67 °C |
| CISH reverse | GGGGGCATTTACCTCCAAGT (SEQ ID NO: 42) | |
| SOCS1 forward | TCCACTGAGGCTGAACGGAT (SEQ ID NO: 43) | 65 °C |
| SOCS1 reverse | CTGAAAGTGCACGCGGATG (SEQ ID NO: 44) | |
| FAS forward | GAAACGTCTGTGAGCCTCTC (SEQ ID NO: 77) | 65 °C |
| FAS reverse | CCTTTGGCTTGGCTTGTCAG (SEQ ID NO: 78) | |

TABLE 4
Indel percentage of candidate genes

| | CISH | SOCS1 | BIM | FAS |
|---|---|---|---|---|
| Group | Indel | Indel | Indel | Indel |
| Scramble | — | — | — | — |
| CISH | 100% | — | — | — |
| SOCS1 | — | 87% | — | — |
| BIM | — | — | 94% | — |
| FAS | — | — | — | 95% |
| CISH + SOCS1 | 100% | 87% | — | — |
| BIM + CISH + SOCS1 | 100% | 83% | 94% | — |
| BIM + CISH + SOCS1 + FAS | 100% | 84% | 90% | 77% |

Example 2. CISH^{-/-}SOCS1^{-/-}DKO and CISH^{-/-}SOCS1^{-/-}BIM^{-/-} (More Specifically, BIM_{EL} and BIM_{L} Deficiency while Preserving BIM_{S}) TKO Enhance the Tumor Cell Killing by KRAS G12V TCR T-iαβT Cells and Persistence of KRAS G12V TCR T-iαβT Cells

### Example 2.1 Serial Killing of KRAS G12V TCR T-iαβT Cells Against HLA-A11-SW620 Cells

KRAS G12V TCR T-iαβT cells could specifically target KRAS G12V, which could be used to verify the anti-tumor potency and persistence of T-iαβT cells after modification. Since human αβT cells are activated in a major histocompatibility complex-dependent manner, HLA-A11 was overexpressed in GFP-SW620 tumor cell line (having KRAS G12V expression) to match with the KRAS G12V TCR in TiPSC-αβT cells.

5×10³ of GFP-HLA-A11-SW620 cells (FenicsBIO (Halethorpe, MD), Cat: CL-1509, an adherent cell line exhibiting epithelial-like morphology that was isolated from the large intestine of a Dukes C colorectal cancer patient) were pre-seeded in a 96-well cell culture plate (Corning, Cat: 3599) overnight. After seeding overnight, the culture supernatant was aspirated. The effector KRAS G12V TCR T-iαβT cells with target gene deficiencies, generated from Example 1, were added into the GFP-HLA-A11-SW620 pre-seeded 96-well cell culture plate in the presence of human IL-2 (0.1 ng/mL) at a 4:1 effector to target cell ratio. The cytotoxicity of KRAS G12V TCR T-iαβT cells with target gene deficiencies was monitored using live-cell imaging and analysis, using the Incucyte® SX5 Live-Cell Analysis System (Sartorius, Göttingen, Germany) through a 4× objective and scheduled 48-hour repeated scanning. Hereafter, the effector KRAS G12V TCR T-iαβT cells with target gene deficiencies were transferred to another GFP-HLA-A11-SW620 pre-seeded 96-well cell culture plate after 48 hours of tumor killing for a further round of killing. The cytotoxicity percentage was calculated by the formula listed below:

a. Cytotoxicity percentage=[1−(Total green fluorescence area at indicated timepoint/Total green fluorescence area at starting timepoint of each round of serial killing)]×100%

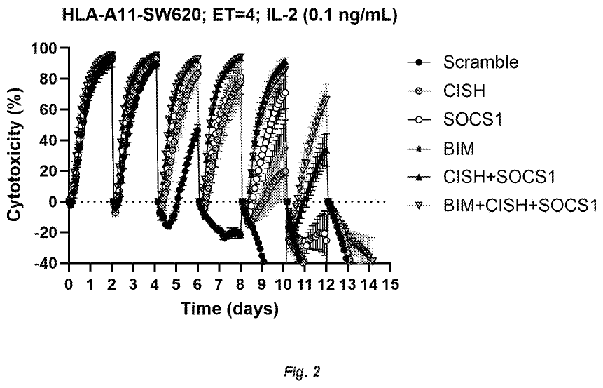

As shown in FIG. 2, scramble sgRNA-nucleofected KRAS G12V TCR T-iαβT cells could exhibit three rounds of killing against the GFP-HLA-A11-SW620 cells in the presence of IL-2 (0.1 ng/mL) at a 4:1 E:T ratio. By contrast, CISH^{-/-} or SOCS1^{-/-} single knockout (SKO) KRAS G12V TCR iαβT cells exhibited greater durable cytotoxicity (five rounds) in the presence of IL-2 (0.1 ng/mL) at a 4:1 E:T ratio. CISH^{-/-}SOCS1^{-/-} double knockout (DKO) KRAS G12V TCR T-iαβT cells exhibited better effects in promoting iαβT cytotoxicity and persistence, especially in the fourth round, fifth round and sixth round of killing, compared with CISH or SOCS1 single knockout T-iαβT cells in the presence of IL-2 (0.1 ng/mL) at a 4:1 E:T ratio (FIG. 2).

Overall, CISH^{-/-}SOCS1^{-/-}DKO KRAS G12V TCR T-iαβT cells or CISH^{-/-}SOCS1^{-/-}BIM^{-/-} (more specifically, BIM_{EL} and BIM_{L} deficiency while preserving BIM_{S}) TKO KRAS G12V TCR T-iαβT cells exhibited better effects in enhanced cytotoxicity and persistence against GFP-HLA-A11-SW620 cells compared with all the other single gene knockout KRAS G12V TCR T-iαβT cells in the 2D serial killing assay platform (FIG. 2).

Example 2.2 Increased IFN-g secretion in CISH^{-/-}SOCS1^{-/-} double knockout (DKO) and BIM^{-/-}CISH^{-/-}SOCS1^{-/-} triple knockout (TKO) KRAS G12V TCR T-iαβT cells

The effector SKO (CISH^{-/-}, SOCS1^{-/-}, BIM^{-/-} or FAS^{-/-}), DKO (CISH^{-/-}/SOCS1^{-/-}) or TKO (BIM^{-/-}/CISH^{-/-}/SOCS1^{-/-}) KRAS G12V TCR T-iαβT cells were co-cultured with 5×10³ of GFP-HLA-A11-SW620 cells (FenicsBIO (Halethorpe, MD), Cat: CL-1509, an adherent cell line exhibiting epithelial-like morphology that was isolated from the large intestine of a Dukes C colorectal cancer patient) in 200 mL of a culture medium in the presence of IL-2 (0.1 ng/mL) at a 4:1 effector to target cell ratio in a 96-well cell culture plate (Corning, Cat: 3599) for 48 hours. After 48 hours of co-culturing, 50 mL of culture supernatant was harvested and stored at −80 °C. The IFN-g in the culture supernatants were quantified using the Lumit® IFN-g (Human) Immunoassay Kit (Promega, Cat: W6040) according to the manufacturer's instructions. The data was acquired using the GloMax® Discover GM3000 Microplate Reader (Promega).

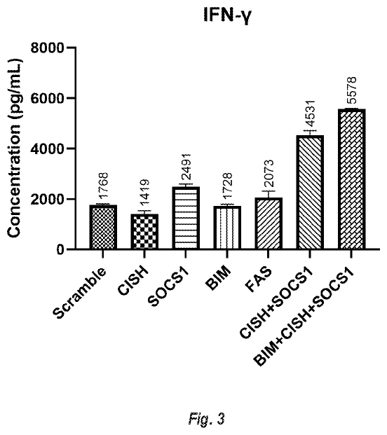

As shown in FIG. 3, the CISH^{-/-}SOCS1^{-/-}DKO and BIM^{-/-}CISH^{-/-}SOCS1^{-/-}TKO KRAS G12V TCR T-iαβT cells secreted higher amounts of IFN-g into the culture supernatant compared with SKO (CISH^{-/-}, SOCS1^{-/-}, BIM^{-/-} or FAS^{-/-}) KRAS G12V TCR T-iαβT cells post co-culturing with GFP-HLA-A11-SW620 cells at a 4:1 E:T ratio (FIG. 3), showing synergistic effect in enhancing IFN-g release.

Example 3. Prolonged survival of BIM^{-/-}CISH^{-/-}SOCS1^{-/-} triple knockout (TKO) KRAS G12V TCR T-iαβT cells post IL-2 deprivation for 8 days

### Example 3.1 Methods

Survival Assay with IL-2 Deprivation

T-iαβT cells with target gene deficiency were harvested on day 10 post activation and washed with 1 mL of cytokine-free culture medium. 3×10⁴ of the harvested T-iαβT cells were resuspended in 100 mL of pre-warmed cytokine-free culture medium. The two-fold culture medium was supplemented with titration of IL-2 (0 and 0.2 ng/mL). 100 mL of the T-iαβT cell suspension (3×10⁴ cells) was mixed with 100 mL of the two-fold culture medium and were seeded in a 96-well cell culture plate (Falcon (Corning, Corning, NY), Cat: 353072) at total volume of 200 mL/well. 240 mL of PBS was added to the outer 36 wells to prevent evaporation of the medium. Seeded T-iαβT cells were incubated at 37 °C. with 5% CO₂ for 2, 4, 6, or 8 days. After IL-2 deprivation, T-iαβT cells were harvested and transferred into a fresh 1.6 mL microcentrifuge tube every two days. Cell viability was determined with FITC-Annexin V (1:200; BioLegend (San Diego, CA), Cat: 640905) and Propidium Iodide (1:2,500; BioLegend, Cat: 421301) staining according to the manufacturer's instructions and analyzed by Attune NxT Flow Cytometer (ThermoFisher Scientific (Waltham, MA)). A live T-iαβT cells was defined as a Annexin V negative-PI negative population analyzed with FlowJo software (BD Biosciences (San Jose, CA)). The relative viability at the indicated timepoint was calculated by normalization to the viability at 0 h timepoint.

Example 3.2 Prolonged Survival of KRAS G12V TCR T-iαβT Cells with BIM KO or TKO

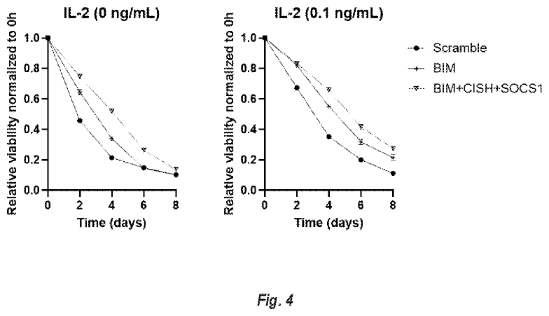

KRAS G12V TCR T-iαβT cells with target gene deficiency (generated according to Example 1) were harvested on day 10 post activation and washed with 1 mL of cytokine-free culture medium. 3×10⁴ of KRAS G12V TCR T-iαβT cells with target gene deficiency were resuspended in 100 mL of pre-warmed cytokine-free culture medium. The culture medium supplemented with a two-fold concentration of IL-2 (0 and 0.2 ng/mL) was prepared and 100 mL of the KRAS G12V TCR T-iαβT cell suspension (with target gene deficiency) was mixed with 100 mL of a prepared culture medium to reach a final IL-2 concentration of 0 and 0.1 ng/mL. KRAS G12V TCR T-iαβT cells with target gene deficiency were seeded in a 96-well cell culture plate (Falcon, Cat: 353072) at a total volume of 200 mL/well, and 240 mL of PBS were added to the outer 36 wells to prevent evaporation of the medium. KRAS G12V TCR T-iαβT cells with target gene deficiency were incubated at 37 °C. with 5% CO₂ for 2-8 days (e.g., 2, 4, 6, or 8 days). After IL-2 deprivation, KRAS G12V TCR T-iαβT cells with target gene deficiency were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune NXT Flow Cytometer (ThermoFisher Scientific). Live KRAS G12V TCR T-iαβT cells with target gene deficiency were defined as an Annexin V negative-PI negative population analyzed with FlowJo software (BD Biosciences). The relative viability of indicated timepoints was calculated by normalization to the viability of the 0 h timepoint. KRAS G12V TCR T-iαβT cells with a BIM_{EL} and BIM_{L} deficiency while preserving BIM_{S} (knockout by BIM sgRNA #5) or CISH^{-/-}SOCS1^{-/-}BIM^{-/-} (more specifically, BIM_{EL} and BIM_{L} deficiency while preserving BIM_{S}) TKO T-iαβT cells exhibited better survival post IL-2 deprivation for 2-8 days (e.g., 2, 4, 6, or 8 days) (FIG. 4).

### Example 3.3 BIM_{S} Splice Variant is Necessary for Prolonging T-iαβT Cell Survival Post IL-15 Deprivation

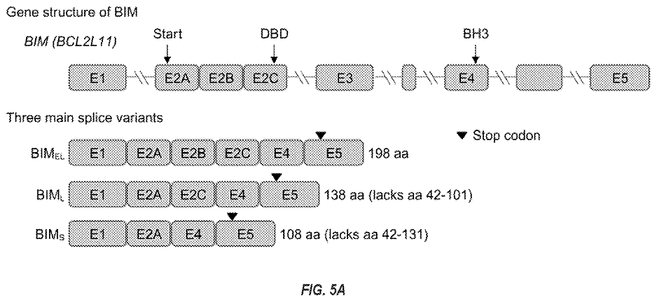

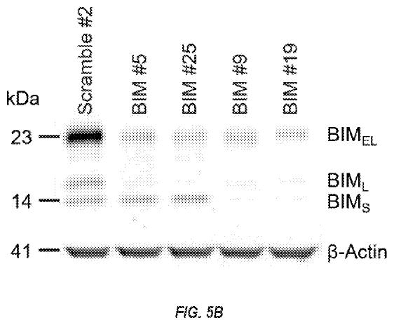

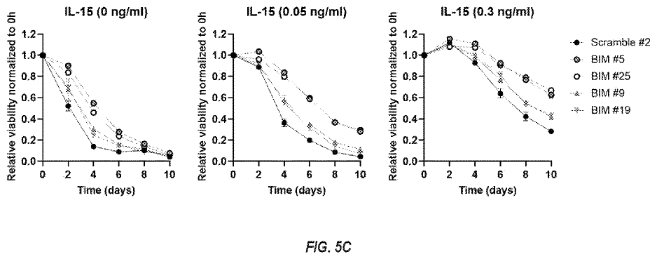

Human BIM gene, also called BCL2L11 (Gene ID: 10018), is transcribed as three splice variants, BIM_{EL} (NM_138621.5; NP_619527.1, containing coding exons E1, E2A, E2B, E2C, E4 and E5), BIM_{L} (NM_006538.5; NP_006529.1, containing coding exons E1, E2A, E2C, E4 and E5) and BIM_{S} (NM_001204106.1; NP_001191035.1, containing coding exons E1, E2A, E4 and E5). FIG. 5A shows gene structure of BIM and three main BIM splice variants expressed in T-iαβT cells. KRAS G12V TCR T-iαβT cells were nucleofected with 200 μmol of Alt-R™ S.p. HiFi Cas9 Nuclease V3 (Integrated DNA Technologies; Cat: 1081060) and 1,500 μmol of synthetic single guide RNA (sgRNAs). We had two synthetic sgRNAs that target BIM exon E2C (BIM #5 (SEQ ID NO: 26) and #25 (SEQ ID NO: 35)) and two synthetic sgRNAs that target BIM exon E2A (BIM #9 (SEQ ID NO: 32) and #19 (SEQ ID NO: 33)). Scramble #2 (SEQ ID NO: 30; a negative control for gene knockout), BIM #5 (SEQ ID NO: 26; targeting BIM_{EL} and BIM_{L} only), BIM #25 (SEQ ID NO: 35; targeting BIM_{EL} and BIM_{L} only), BIM #9 (SEQ ID NO: 32; targeting all three BIM splice variants) or BIM #19 (SEQ ID NO: 33; targeting all three BIM splice variants), as listed in Table 5, were nucleofected by Lonza P3 Primary Cell 4D-Nucleofector® X Kit L (Lonza, Cat: V4XP-3024) and Lonza 4D Nucleofector® Core and X Unit (Lonza, Cat: AAF-1003X). Western blotting results showed that BIM_{EL} and BIM_{L} were knocked out in BIM #5- and BIM #25-nucleofected T-iαβT cells and all three BIM splice variants were knocked out in BIM #9- and BIM #19-nucleofected T-iαβT cells. There were some background bands for BIM_{EL} because the nucleofection efficiency was not 100%, and the Western blot samples were from pool populations (FIG. 5B).

T-iαβT cells were harvested on day 4 post nucleofection and washed with 1 mL of cytokine-free culture medium. 3×10⁴ of T-iαβT cells were resuspended in 100 mL of pre-warmed cytokine-free culture medium. The culture medium supplemented with a two-fold concentration of IL-15 (0, 0.1 and 0.6 ng/mL) was prepared, and 100 mL of the T-iαβT cell suspension was mixed with 100 mL of a prepared culture medium to reach a final IL-15 concentration of 0, 0.05 and 0.3 ng/mL. T-iαβT cells were seeded in a 96-well cell culture plate (Falcon, Cat: 353072) at a total volume of 200 mL/well, and 240 mL of PBS were added to the outer 36 wells to prevent evaporation of the medium. T-iαβT cells were incubated at 37 °C. with 5% CO₂ for 2-12 days (2, 4, 6, 8 or 10 days). After IL-15 deprivation, T-iαβT cells were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune™ NXT Flow Cytometer (ThermoFisher Scientific). Live T-iαβT cells were defined as Annexin V negative-PI negative population analyzed with FlowJo™ software (BD Biosciences). The relative viability of indicated timepoints were calculated by normalization to the viability of the 0 h timepoint.

Overall, T-iαβT cells that lack BIM_{EL} and BIM_{L} splice variants but retain the BIM_{S} splice variant (BIM #5 or BIM #25) exhibited better survival compared with T-iαβT cells lacking all three BIM splice variants (BIM #9 or BIM #19) post IL-15 deprivation for 10 days (FIG. 5C).

TABLE 5
sgRNA sequence for targeting BIM splice variants with Cas9 nuclease

| | Name | | Targeting/guide | Target | |
|---|---|---|---|---|---|
| Target | of | | sequence in | sequence in | Target BIM |
| Gene | sgRNA | Synthetic sgRNA sequence | sgRNA | target gene | isoform |
| — | Scramble | mG*mC*mA*rCrUrCrArCrArUr | GCACTCACATC | TGATGTAGC | / |
| | #2 | CrGrCrUrArCrArUrCrArGrUrUr | GCTACATCA | GATGTGAGT | |
| | | UrUrArGrArGrCrUrArGrArArA | (SEQ ID NO: 1) | GC | |
| | | rUrArGrCrArArGrUrUrArArAr | | (SEQ ID NO: | |
| | | ArUrArArGrGrCrUrArGrUrCrC | | 13) | |
| | | rGrUrUrArUrCrArArCrUrUrGr | | | |
| | | ArArArArArGrUrGrGrCrArCrC | | | |
| | | rGrArGrUrCrGrGrUrGrCmU*m | | | |
| | | U*mU*rU (SEQ ID NO: 30) | | | |
| BIM | BIM #5 | mU*mA*mC*rCrCrArUrUrGrCr | TACCCATTGCA | CTATCTCAG | Target |
| (BCL2 | | ArCrUrGrArGrArUrArGrGrUrU | CTGAGATAG | TGCAATGGG | sequence is in |
| L11) | | rUrUrArGrArGrCrUrArGrArAr | (SEQ ID NO: 2) | TA | exon 2C, |
| | | ArUrArGrCrArArGrUrUrArArA | | (SEQ ID NO: | resulting in |
| | | rArUrArArGrGrCrUrArGrUrCr | | 14) | knockout of |
| | | CrGrUrUrArUrCrArArCrUrUrG | | | BIMEL and |
| | | rArArArArArGrUrGrGrCrArCr | | | BIML |
| | | CrGrArGrUrCrGrGrUrGrCmU* | | | |
| | | mU*mU*rU (SEQ ID NO: 26) | | | |
| | BIM #9 | mA*mG*mU*rUrCrUrGrArGrU | AGTTCTGAGTG | TCTCGGTCA | Target |
| | | rGrUrGrArCrCrGrArGrArGrUr | TGACCGAGA | CACTCAGA | sequence is in |
| | | UrUrUrArGrArGrCrUrArGrArA | (SEQ ID NO: 6) | ACT | exon 2A, |
| | | rArUrArGrCrArArGrUrUrArAr | | (SEQ ID NO: | resulting in |
| | | ArArUrArArGrGrCrUrArGrUrC | | 18) | knockout of |
| | | rCrGrUrUrArUrCrArArCrUrUr | | | BIMEL, BIML |
| | | GrArArArArArGrUrGrGrCrArC | | | and BIMs |
| | | rCrGrArGrUrCrGrGrUrGrCmU* | | | |
| | | mU*mU*rU (SEQ ID NO: 32) | | | |
| | BIM | mG*mC*mC*rUrCrCrCrCrArGr | GCCTCCCCAGC | CAGGTCTGA | Target |
| | #19 | CrUrCrArGrArCrCrUrGrGrUrUr | TCAGACCTG | GCTGGGGA | sequence is in |
| | | UrUrArGrArGrCrUrArGrArArA | (SEQ ID NO: 7) | GGC | exon 2A, |
| | | rUrArGrCrArArGrUrUrArArAr | | (SEQ ID NO: | resulting in |
| | | ArUrArArGrGrCrUrArGrUrCrC | | 19) | knockout of |
| | | rGrUrUrArUrCrArArCrUrUrGr | | | BIMEL, BIML |
| | | ArArArArArGrUrGrGrCrArCrC | | | and BIMs |
| | | rGrArGrUrCrGrGrUrGrCmU*m | | | |
| | | U*mU*rU (SEQ ID NO: 33) | | | |
| | BIM | mG*mG*mC*rCrUrGrGrCrArA | GGCCTGGCAAG | CAAGTCCTC | Target |
| | #24 | rGrGrArGrGrArCrUrUrGrGrUr | GAGGACTTG | CTTGCCAGG | sequence is in |
| | | UrUrUrArGrArGrCrUrArGrArA | (SEQ ID NO: 8) | CC | exon 2C, |
| | | rArUrArGrCrArArGrUrUrArAr | | (SEQ ID NO: | resulting in |
| | | ArArUrArArGrGrCrUrArGrUrC | | 20) | knockout of |
| | | rCrGrUrUrArUrCrArArCrUrUr | | | BIMEL and |
| | | GrArArArArArGrUrGrGrCrArC | | | BIML |
| | | rCrGrArGrUrCrGrGrUrGrCmU* | | | |
| | | mU*mU*rU (SEQ ID NO: 34) | | | |
| | BIM | mU*mG*mG*rUrUrGrArArGrG | TGGTTGAAGGC | CCTTGCCAG | Target |
| | #25 | rCrCrUrGrGrCrArArGrGrGrUr | CTGGCAAGG | GCCTTCAAC | sequence is in |
| | | UrUrUrArGrArGrCrUrArGrArA | (SEQ ID NO: 9) | CA | exon 2C, |
| | | rArUrArGrCrArArGrUrUrArAr | | (SEQ ID NO: | resulting in |
| | | ArArUrArArGrGrCrUrArGrUrC | | 21) | knockout of |
| | | rCrGrUrUrArUrCrArArCrUrUr | | | BIMEL and |
| | | GrArArArArArGrUrGrGrCrArC | | | BIML |
| | | rCrGrArGrUrCrGrGrUrGrCmU* | | | |
| | | mU*mU*rU (SEQ ID NO: 35) | | | |

Example 4. Determining the Editing Efficiency of 3 Different crRNAs Through CRISPR/MAD7-Mediated Gene Editing.
Electroporation of MAD7 Ribonucleoproteins (RNP) in iPSCs

Electroporation was done using Lonza P3 Primary Cell 4D-Nucleofector® X Kit S (Lonza, V4XP-3032) and Lonza 4D-Nucleofector® System (Lonza, AAF-1003X) according to the manufacture's protocol. For editing of each individual gene, corresponding MAD7 ribonucleoprotein (RNP) was prepared by incubating 30 μmol of MAD7 (GenScript; Piscataway, NJ) and 150 μmol of crRNA targeting the BIM (BCL2L11) (SEQ ID NO: 10), SOCS1 (SEQ ID NO: 11), or CISH (SEQ ID NO: 12) (customized by IDT; see Table 6) in 10 μL of Lonza P3 Primary Nucleofector® solution for 15-20 min at room temperature. For each electroporation reaction, 3×10⁵ to 5×10⁵ iPSCs were first resuspended in the 10 μL of Lonza P3 Primary Nucleofector® solution, then combined with 10 μL of the incubated MAD7 RNP to a final reaction volume of 20 μL. Resuspended cells with RNP were then transferred into Nucleocuvette Strips and electroporation was performed using the CA-137 program. After completion, cells were seeded into vitronectin (VTN)-coated culture plates containing StemFlex™ medium (ThermoFisher, A3349401) with RevitaCell™ (ThermoFisher, A2644501). The second-round of electroporation was performed 48 hours later, and cells were collected for analysis 2 days after the second electroporation. Single clone selection was then performed on the CISH, SOCS1, or BIM edited cells.

TABLE 6
Full-length crRNA sequences and their corresponding target sequences for MAD7
endonuclease at 3 indicated genomic loci.

| | | | Targeting/guide | | |
|---|---|---|---|---|---|
| Target | Name of | | sequence in | Target sequence | Target |
| gene | crRNA | Full crRNA sequence | crRNA | in target gene | exon |
| BIM | BIM | mG*mU*mC*rArArArArGrArCrCrUr | ACACAGACAGG | GTGCTGGGCTC | Exon 2C |
| (BCL2L11) | crRNA | UrUrGrGrArArUrUrUrCrUrArCrUrCr | AGCCCAGCAC | CTGTCTGTGT | (designed |
| | | UrUrGrUrArGrArUrArCrArCrArGrA | (SEQ ID | (SEQ ID | to |
| | | rCrArGrGrArGrCrCrCrAmG*mC*m | NO: 10) | NO: 22) | retain |
| | | A*rC (SEQ ID NO: 36) | | | BIMs) |
| SOCS1 | SOCS1 | mG*mU*mC*rArArArArGrArCrCrUr | ACCTGGATGGC | TCTCGCGGCTG | Exon 2 |
| | crRNA | UrUrGrGrArArUrUrUrCrUrArCrUrCr | AGCCGCGAGA | CCATCCAGGT | |
| | | UrUrGrUrArGrArUrArCrCrUrGrGrA | (SEQ ID | (SEQ ID | |
| | | rUrGrGrCrArGrCrCrGrCmG*mA*m | NO: 11) | NO: 23) | |
| | | G*rA (SEQ ID NO: 37) | | | |
| CISH | CISH | mG*mU*mC*rArArArArGrArCrCrUr | GGTGTACAGCA | CACCAGCCACT | Exon 3 |
| | crRNA | UrUrGrGrArArUrUrUrCrUrArCrUrCr | GTGGCTGGTG | GCTGTACACC | |
| | | UrUrGrUrArGrArUrGrGrUrGrUrArC | (SEQ ID | (SEQ ID | |
| | | rArGrCrArGrUrGrGrCrUmG*mG*m | NO: 12) | NO: 24) | |
| | | U*rG (SEQ ID NO: 38) | | | |

### Editing Efficiency Determination by ICE Analysis

Genomic DNA of edited cells was obtained by QuickExtract DNA Extraction Solution (LGC, SS000035-D2) or QIAamp DNA Mini Kit (Qiagen (Hilden, Germany), QIA-51306) as per manufacture's protocol. Target locus-specific DNA fragments were amplified using KAPA HiFi HotStart PCR Kit (Roche, KR0369) with specific primer sets (See Table 7). The PCR products were then sequenced using Sanger sequencing by MISSION BIOTECH. Sequencing primers used for each locus are listed in Table 7. The editing efficiency was determined by analyzing the indel formation based on the sequencing results of amplified DNA fragments using the Synthego ICE Analysis tool (Synthego)

TABLE 7
PCR and Sanger sequencing primers used in
analyzing editing efficiency of target loci.

| | PCR primer - Forward | PCR primer - Reverse | Sequencing primer |
|---|---|---|---|
| IBIM | ACCGAGAAGGTAGACAATT | AGTTTGACACATCCTCCATT | AATCACGGAGGTGAAGGGG |
| | GCAGC (SEQ ID NO: 45) | CCCA (SEQ ID NO: 46) | A (SEQ ID NO: 47) |
| SOCS1 | GCCCCTTCTGTAGGATGGTA | GGGGAAGGAGCTCAGGTAG | AGCACACAACCAGGTGGCA |
| | GC (SEQ ID NO: 48) | TCG (SEQ ID NO: 49) | G (SEQ ID NO: 50) |
| CISH | GGTTCCATTACGGCCAGCGA | GGCCAGCTGCCAGAGGTAT | CGCACCCCAGCTACCTGTTC |
| | (SEQ ID NO: 51) | G (SEQ ID NO: 52) | (SEQ ID NO: 53) |

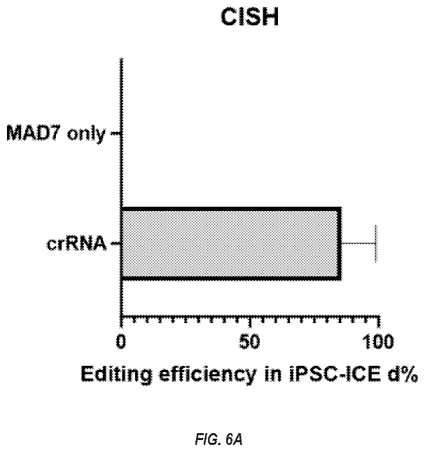

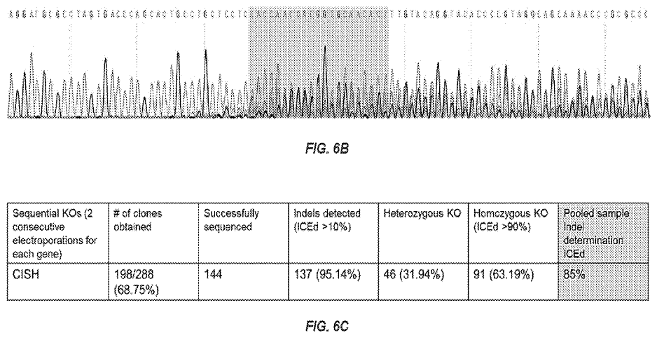

As shown in FIG. 6A, successful CRISPR/MAD7-mediated CISH crRNA gene editing was observed with 85% indel (small insertion/deletion resulted from DNA cleavage and repair) formation at the CISH locus; in contrast, there was no indel observed in the MAD7 protein only control. The indel can be visualized in a Sanger sequencing chromatogram as patterns of mixed peaks beginning around the targeting site (shaded area, FIG. 6B). As summarized in FIG. 6C, single clone selection was performed on pools of CISH crRNA edited cells to obtain clones arising from a single cell. Out of all obtained clones, 144 clones were successfully sequenced. Among the 144 clones, CISH editing was observed in 137 (95%), which was significantly higher than observed in the pooled population. Further analysis suggested that 46 of the 137 clones were heterozygous knock outs (with one allele of CISH edited), and the remaining 96 were homozygous knock outs (both alleles edited, complete knock out), again indicating successful CRISPR/MAD7-mediated editing with CISH crRNA.

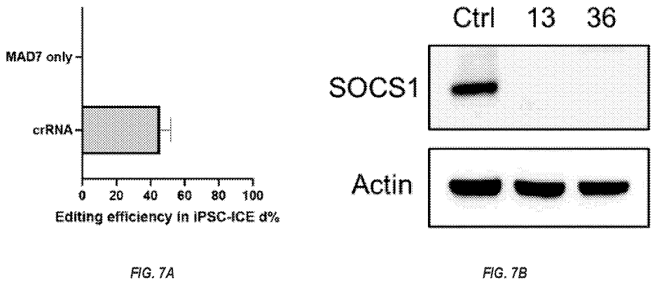

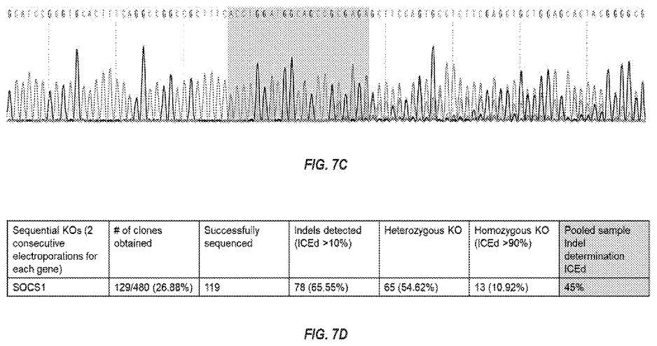

As shown in FIG. 7A, successful CRISPR/MAD7-mediated SOCS1 crRNA editing was observed with 45% indel formation at the SOCS1 locus; in contrast, there was no indel observed in the MAD7 protein only control. The indel can be visualized in a Sanger sequencing chromatogram as patterns of mixed peaks beginning around the targeting site (shaded area, FIG. 7C). As summarized in FIG. 7D, single clone selection was performed on pools of SOCS1 edited cells to obtain clones arising from a single cell. Out of all obtained clones, 119 clones were successfully sequenced. Among the 119 clones, SOCS1 editing was observed in 78 clones (65.55%). Further analysis suggested that 65 out of 78 clones were heterozygous knock outs (with one allele of SOCS1 being edited) and the remaining 13 were homozygous knock outs (both alleles edited, complete knock out). These results again demonstrate successful CRISPR/MAD7-mediated editing with SOCS1 crRNA. To determine the disruption of SOCS1 gene product after editing, western blot analysis was performed on two of the homozygous knockout clones (clone numbers 13 and 36) obtained during the single clone selection process. FIG. 7B shows the complete depletion of SOCS1 protein in both clones, suggesting the translation of successful gene editing/knockout at the protein level.

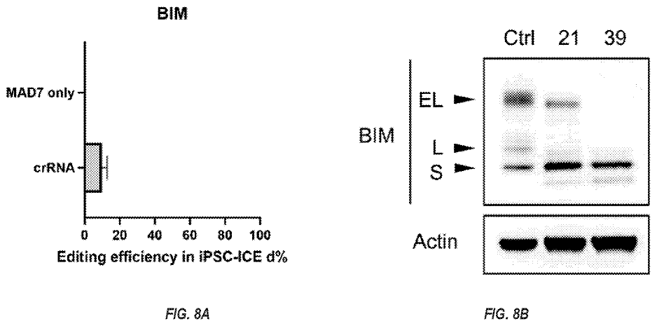

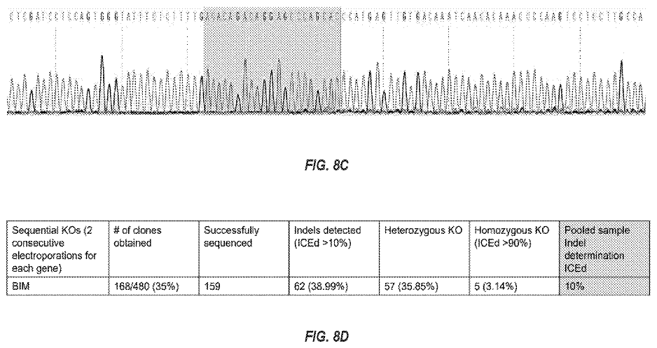

As shown in FIG. 8A, successful editing was observed with BIM crRNA in CRISPR/MAD7-mediated gene editing, evidenced by 10% indel formation at the BIM locus; in contrast, there was no indel observed in the MAD7 protein only control. The indel can be visualized in a Sanger sequencing chromatogram as patterns of mixed peaks beginning around the targeting site (shaded area, FIG. 8C). As summarized in FIG. 8D, single clone selection was performed on pools of BIM edited cells to obtain clones arising from a single cell. Out of all obtained clones, 159 clones were successfully sequenced, among which, BIM editing was observed in 62 clones (38.99%). Further analysis suggested that 57 of 62 clones were heterozygous knock outs (with one allele of BIM being edited) and the remaining 5 clones were homozygous knock outs (both alleles edited, complete knock out). This again demonstrates the successful CRISPR/MAD7-mediated editing with BIM crRNA. To determine the disruption of the BIM gene product after editing, a western blot analysis was performed on one heterozygous knockout clone (clone number 21) and one homozygous knockout clone (clone number 39) obtained during the single clone selection process. As shown in FIG. 8B, a partial depletion of the BIM_{EL} and BIM_{L} protein was observed in clone 21, whereas clone 39 obtained a complete depletion of both BIM_{EL} and BIM_{L} while retaining BIM_{S}, establishing the effects of successful gene editing/knockout at the protein level. Note that BIM_{S} remained in both clones, as the BIM crRNA was designed to deplete BIM_{EL} and BIM_{L} splice forms but to retain BIM_{S}.

### Single Clone Selection

48 h after gene editing, CISH, SOCS1, or BIM edited iPSCs were single-cell seeded into VTN-coated 96-well plates either by limiting dilution or single cell sorting. Limiting dilution was done by resuspending cells to a density of 5 cells/mL in StemFlex™ medium (ThermoFisher, A3349401) with RevitaCell™ (ThermoFisher, A2644501) and seeding 100 μL of cell suspension into each well of the 96-well plates. Seeding at this low density of 0.5 cells/well ensures the isolation of single cells in individual wells, thus establishing single clones. Alternatively, single cell sorting was performed using a BD FACSAria™ III Sorter (BD Biosciences). Cells were left undisturbed for 48 h to 72 h after seeding to increase cell viability. Regular medium changes were performed every 2-3 days thereafter for 2 weeks or until cell colonies' diameter was larger than 800 μm for collection or further expansion.

### Western Blot

A whole cell lysate was obtained from control, SOCS1, or BIM edited clones for examining the depletion of targeted gene products. Cells were lysed in RIPA buffer (Millipore, 20-188) on ice for 20 min, then centrifuged at 20,000×g for 15 min at 4 °C. to remove insoluble debris. Next, the protein concentration was determined using a Protein Assay Dye Reagent (Bio-Rad, 5000006). Equal amounts of protein from each sample were subjected to SDS-PAGE using mPAGE 4-20% Bis-Tris Precast Gel (Millipore, MP42G12). Subsequently, the proteins in the gels were transferred to a Power Blotter Select Transfer PVDF membrane (Invitrogen, PB5340). After blocking the membrane in 5% milk in TBST for 1 h at room temperature, primary antibodies (BIM, 1:1,000, Cell Signaling Technology, 2933S; SOCS1, 1:1,000, Cell Signaling Technology, 68631S; Beta-actin, 1:5,000, Santa Cruz Biotechnology, sc-47778 HRP) were applied to corresponding blots and incubated O/overnight at 4 °C. The membranes were then washed with TBST before incubating with peroxidase conjugated goat anti-rabbit IgG antibody (1:5,000, Sigma-Aldrich, AP132P) for 1 h at room temperature. The blots were finally washed with TBST, and a chemiluminescent signal was developed using SuperSignal™ West Pico PLUS Chemiluminescent Substrate (ThermoFisher Scientific, 34580).

TABLE 8
Summary of targeting/guide sequences in sgRNA/crRNA and
target sequences in target gene

| SEQ ID NO | Name | SEQUENCE |
|---|---|---|
| SEQ ID NO: 1 | Targeting sequence in | GCACTCACATCGCTACATCA |
| | Scramble #2 | |
| SEQ ID NO: 2 | Targeting sequence in | TACCCATTGCACTGAGATAG |
| | BIM # 5 | |
| SEQ ID NO: 3 | Targeting sequence in | AGGCCACATAGTGCTGCACA |
| | CISH #3 | |
| SEQ ID NO: 4 | Targeting sequence in | TGTACAGCAGTGGCTGGTGG |
| | CISH #4 | |
| SEQ ID NO: 5 | Targeting sequence in | AGTAGAATCCGCAGGCGTCC |
| | SOCS1 sgRNA | |
| SEQ ID NO: 6 | Targeting sequence in | AGTTCTGAGTGTGACCGAGA |
| | BIM #9 | |
| SEQ ID NO: 7 | Targeting sequence in | GCCTCCCCAGCTCAGACCTG |
| | BIM #19 | |
| SEQ ID NO: 8 | Targeting sequence in | GGCCTGGCAAGGAGGACTTG |
| | BIM #24 | |
| SEQ ID NO: 9 | Targeting sequence in | TGGTTGAAGGCCTGGCAAGG |
| | BIM #25 | |
| SEQ ID NO: 10 | Targeting sequence in | ACACAGACAGGAGCCCAGCAC |
| | BIM crRNA (MAD7) | |
| SEQ ID NO: 11 | Targeting sequence in | ACCTGGATGGCAGCCGCGAGA |
| | SOCS1 crRNA (MAD7) | |
| SEQ ID NO: 12 | Targeting sequence in | GGTGTACAGCAGTGGCTGGTG |
| | CISH crRNA (MAD7) | |
| SEQ ID NO: 13 | Target sequence by | TGATGTAGCGATGTGAGTGC |
| | Scramble #2 sgRNA | |
| SEQ ID NO: 14 | Target sequence in BIM | CTATCTCAGTGCAATGGGTA |
| | by BIM # 5 sgRNA | |
| SEQ ID NO: 15 | Target sequence in CISH | TGTGCAGCACTATGTGGCCT |
| | by CISH #3 sgRNA | |
| SEQ ID NO: 16 | Target sequence in CISH | CCACCAGCCACTGCTGTACA |
| | by CISH #4 sgRNA | |
| SEQ ID NO: 17 | Target sequence in | GGACGCCTGCGGATTCTACT |
| | SOCS1 by SOCS1 | |
| | sgRNA | |
| SEQ ID NO: 18 | Target sequence in BIM | TCTCGGTCACACTCAGAACT |
| | by BIM #9 sgRNA | |
| SEQ ID NO: 19 | Target sequence in BIM | CAGGTCTGAGCTGGGGAGGC |
| | by BIM #19 sgRNA | |
| SEQ ID NO: 20 | Target sequence in BIM | CAAGTCCTCCTTGCCAGGCC |
| | by BIM #24 sgRNA | |
| SEQ ID NO: 21 | Target sequence in BIM | CCTTGCCAGGCCTTCAACCA |
| | by BIM #25 sgRNA | |
| SEQ ID NO: 22 | Target sequence in BIM | GTGCTGGGCTCCTGTCTGTGT |
| | by BIM crRNA (MAD7) | |
| SEQ ID NO: 23 | Target sequence in | TCTCGCGGCTGCCATCCAGGT |
| | SOCS1 by SOCS1 | |
| | crRNA (MAD7) | |
| SEQ ID NO: 24 | Target sequence in CISH | CACCAGCCACTGCTGTACACC |
| | by CISH crRNA (MAD7) | |

### Example 5. FAS Deficiency

### Example 5.1 FAS Deficiency Resists FASL-Induced Apoptosis and Prolongs KRAS G12V TCR T-iαβT Cell Survival

Survival Assay with IL-2 Deprivation in the Presence of FAS Ligand

3×10⁴ of control (nucleofection with scramble sgRNA #2) or FAS-deficient KRAS G12V TCR T-iαβT cells were resuspended in 200 μL of pre-warmed cytokine-free culture medium. Both scramble control and FAS-deficient KRAS G12V TCR T-iαβT cells were seeded separately in a 96-well cell culture plate (Falcon, Cat: 353072), coated with FAS Ligand (Peprotech, Cat: 310-03H) at 20 mg/mL overnight, and 240 μL of PBS were added to the outer 36 wells to prevent evaporation of the medium. KRAS G12V TCR T-iαβT cells were incubated at 37 °C. with 5% CO₂ for 2-8 days (e.g., 2, 4, 6, or 8 days). After IL-2 deprivation, KRAS G12V TCR T-iαβT cells were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune NxT Flow Cytometer (ThermoFisher Scientific). Live KRAS G12V TCR T-iαβT cells were defined as an Annexin V negative-PI negative population analyzed with FlowJo software (BD Biosciences). The relative viability of indicated timepoints was calculated by normalization to the viability of the 0 h timepoint.

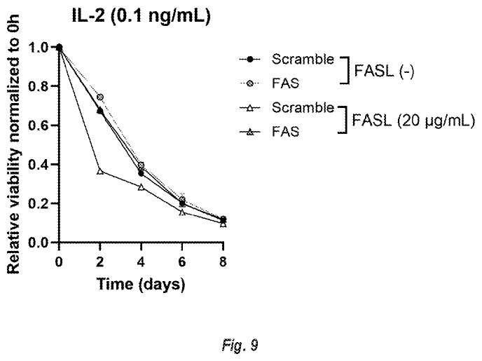

As shown in FIG. 9, there was a comparable survival between control and FAS-deficient KRAS G12V TCR T-iαβT cells, which were deprived of IL-2 at a concentration of 0.1 ng/mL in the absence of FASL for 2 to 8 days. In the presence of FASL (20 mg/mL), there was a further decrease in survival in control KRAS G12V TCR T-iαβT cells after IL-2 deprivation at a concentration of 0.1 ng/mL for 2 to 8 days, especially on day 2 post IL-2 deprivation, compared to the survival of FAS-deficient KRAS G12V TCR T-iαβT cells. By contrast, there was a comparable survival in FAS-deficient regardless of FASL stimulation post IL-2 deprivation at a concentration of 0.1 ng/mL for 2-8 days (FIG. 9).

Overall, FAS-deficient KRAS G12V TCR T-iαβT cells exhibited prolonged survival post FASL-induced apoptosis in the absence of IL-2 for 2-8 days (FIG. 9).

Example 5.2 FAS-Deficient KRAS G12V TCR iαβT Cells Exhibit Enhanced Durable Cytotoxicity Against FASL-Overexpressing HLA-A11-SW620 Cells

### Serial Killing Assay of KRAS G12V TCR T-iαβT Cells Against FASL-Overexpressing HLA-A11 SW620 Cells

To address the impact of FAS ligand (FASL)-induced apoptosis on durable cytotoxicity of KRAS G12V TCR iαβT cells, we knocked out the FAS receptor and overexpressed the FASL in the GFP-HLA-A11-SW620 tumor cells (GFP-HLA-A11-SW620-FASL). 5×10³ of GFP-HLA-A11-SW620-FASL cells were pre-seeded in a 96-well cell culture plate (Corning, Cat: 3599) overnight. After seeding overnight, the culture supernatant was aspirated. The effector control or FAS single knockout (SKO) KRAS G12V TCR T-iαβT cells, generated from Example 1, were added into the GFP-HLA-A11-SW620-FASL pre-seeded 96-well cell culture plate in 200 mL of a culture medium in the presence of human IL-2 (1 ng/mL) at a 4:1 effector to target cell ratio. The cytotoxicity of KRAS G12V TCR T-iαβT cells was monitored using live-cell imaging and analysis, using the Incucyte® SX5 Live-Cell Analysis System (Sartorius, Göttingen, Germany) through a 4× objective and scheduled 48-hour repeated scanning. Hereafter, the effector KRAS G12V TCR T-iαβT cells with target gene deficiencies were transferred to another GFP-HLA-A11-SW620-FASL pre-seeded 96-well cell culture plate after 48 hours of tumor killing for a further round of killing. The cytotoxicity percentage was calculated by the formula listed below:

a. Cytotoxicity percentage=[1−(Total green fluorescence area at indicated timepoint/Total green fluorescence area at starting timepoint of each round of serial killing)]×100%

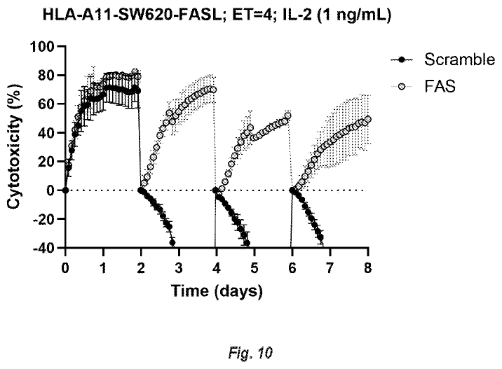

As shown in FIG. 10, scramble sgRNA-nucleofected KRAS G12V TCR iαβT cells could exhibit only one round of killing against the GFP-HLA-A11-SW620-FASL cells in the presence of IL-2 (1 ng/mL) at a 4:1 E:T ratio. By contrast, FAS^{-/-} single knockout (SKO) KRAS G12V TCR iαβT cells significantly exhibited enhanced durable cytotoxicity (four rounds) in the presence of IL-2 (0.1 ng/mL) at a 4:1 E:T ratio (FIG. 10).

Overall, the FAS deficiency enhanced durable cytotoxicity of KRAS G12V TCR iαβT cells against FASL-overexpressing HLA-All SW620 in the serial killing assay platform (FIG. 10).

Example 5.3 FAS Deficiency Protects Functionally Enhanced TKO iαβT Cells from FASL-Induced Apoptosis
Survival Assay with IL-2 Deprivation in the Presence of FAS Ligand

3×10⁴ of TKO (BIM^{-/-}/CISH^{-/-}/SOCS1^{-/-}) or QKO (BIM^{-/-}/CISH^{-/-}/SOCS1^{-/-}FAS^{-/-}) KRAS G12V TCR T-iαβT cells were resuspended in 200 μL of pre-warmed cytokine-free culture medium. Both TKO and QKO KRAS G12V TCR T-iαβT cells were seeded separately in a 96-well cell culture plate (Falcon, Cat: 353072), coated with FAS Ligand (Peprotech, Cat: 310-03H) at 20 mg/mL overnight, and 240 μL of PBS were added to the outer 36 wells to prevent evaporation of the medium. Seeded T-iαβT cells were incubated at 37 °C. with 5% CO₂ for 2-8 days (e.g., 2, 4, 6, or 8 days). After IL-2 deprivation, T-iαβT cells were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune NxT Flow Cytometer (ThermoFisher Scientific). Live T-iαβT cells were defined as an Annexin V negative-PI negative population analyzed with FlowJo software (BD Biosciences). The relative viability of indicated timepoints was calculated by normalization to the viability of the 0 h timepoint.

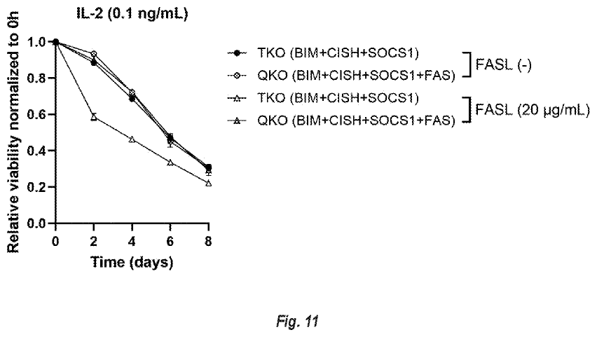

As shown in FIG. 11, there was a comparable survival between TKO (BIM^{-/-}/CISH^{-/-}/SOCS1^{-/-}) and QKO (BIM^{-/-}/CISH^{-/-}/SOCS1^{-/-}FAS^{-/-}) KRAS G12V TCR T-iαβT cells, which were deprived of IL-2 at a concentration of 0.1 ng/mL in the absence of FASL for 2 to 8 days. In the presence of FASL (20 mg/mL), there was a further decrease in survival of TKO (BIM^{-/-}/CISH^{-/-}/SOCS1^{-/-}) KRAS G12V TCR T-iαβT cells post IL-2 deprivation for 2-8 days, when compared to the survival of QKO KRAS G12V TCR T-iαβT cells. By contrast, there was a comparable survival of QKO (BIM^{-/-}/CISH^{-/-}/SOCS1^{-/-}FAS^{-/-}) KRAS G12V TCR T-iαβT cells regardless of FASL stimulation post IL-2 deprivation at a concentration of 0.1 ng/mL for 2-8 days (FIG. 11).

Overall, FAS-deficient and functionally enhanced QKO (BIM^{-/-}/CISH^{-/-}/SOCS1^{-/-}FAS^{-/-}) KRAS G12V TCR T-iαβT cells exhibited prolonged survival post FASL-induced apoptosis in the absence of IL-2 for 2-8 days (e.g., 2, 4, 6 or 8 days) (FIG. 11).

Example 5.4 FAS Deficiency Enhances Durable Cytotoxicity in Functionally Enhanced TKO KRAS G12V TCR T-iαβT Cells Against FASL-Overexpressing HLA-A11-SW620 Cells Serial Killing Assay of KRAS G12V TCR T-iαβT Cells Against FASL-Overexpressing HLA-A11 SW620 Cells

5×10³ of GFP-HLA-A11-SW620-FASL cells were pre-seeded in a 96-well cell culture plate (Corning, Cat: 3599) overnight. After seeding overnight, the culture supernatant was aspirated. The effector TKO or QKO KRAS G12V TCR T-iαβT cells, generated from Example 1, were added into the pre-seeded 96-well cell culture plate in 200 mL of a culture medium in the presence of human IL-2 (0.1 ng/mL) at a 4:1 effector to target cell ratio. The cytotoxicity of KRAS G12V TCR T-iαβT cells with target gene deficiencies was monitored using live-cell imaging and analysis, using the Incucyte® SX5 Live-Cell Analysis System (Sartorius, Göttingen, Germany) through a 4× objective and scheduled 48-hour repeated scanning. Hereafter, the effector KRAS G12V TCR T-iαβT cells with target gene deficiencies were transferred to another GFP-HLA-A11-SW620-FASL pre-seeded 96-well cell culture plate after 48 hours of tumor killing for a further round of killing. The cytotoxicity percentage was calculated by the formula listed below:

a. Cytotoxicity percentage=[1−(Total green fluorescence area at indicated timepoint/Total green fluorescence area at starting timepoint of each round of serial killing)]×100%

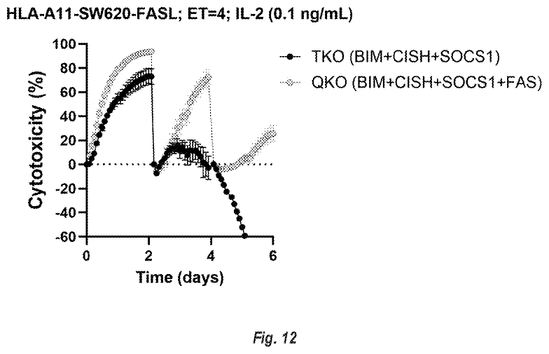

As shown in FIG. 12, TKO (BIM^{-/-}/CISH^{-/-}/SOCS1^{-/-}) KRAS G12V TCR T-iαβT cells could exhibit only one round of killing against the GFP-HLA-A11-SW620-FASL cells in the presence of IL-2 (0.1 ng/mL) at a 4:1 E:T ratio. By contrast, QKO (BIM^{-/-}/CISH^{-/-}/SOCS1^{-/-}FAS^{-/-}) KRAS G12V TCR T-iαβT cells exhibited greater cytotoxicity and enhanced durable tumor killing (3 rounds) against the GFP-HLA-A11-SW620-FASL cells at a 4:1 E:T ratio in the presence of IL-2 (0.1 ng/mL) (FIG. 12).

Overall, the FAS deficiency of the TKO (BIM^{-/-}/CISH^{-/-}/SOCS1^{-/-}) KRAS G12V TCR T-iαβT cells enhanced cytotoxicity and persistence against FASL-overexpressing HLA-A11-SW620 cells compared with the QKO (BIM^{-/-}/CISH^{-/-}/SOCS1^{-/-}FAS^{-/-}) KRAS G12V TCR T-iαβT cells in the serial killing assay platform (FIG. 12).

### Example 5.5. FAS Deficiency Increases IFN-g Secretion in Functionally Enhanced TKO KRAS G12V TCR T-iαβT Cells Post Killing Against FASL-Overexpressing HLA-A11-SW620 Cells

The effector FAS SKO (FAS^{-/-}), TKO (BIM^{-/-}/CISH^{-/-}/SOCS1^{-/-}) or QKO (BIM^{-/-}/CISH^{-/-}/SOCS1^{-/-}FAS^{-/-}) KRAS G12V TCR T-iαβT cells were co-cultured with 5×10³ of GFP-HLA-A11-SW620-FASL cells in 200 mL of a culture medium in the presence of IL-2 (0.1 ng/mL) at a 4:1 effector to target cell ratio in a 96-well cell culture plate (Corning, Cat: 3599) for 48 hours. After 48 hours of co-culturing, 50 mL of culture supernatant was harvested and stored at −80 °C. The IFN-g in the culture supernatants were quantified using the Lumit® IFN-g (Human) Immunoassay Kit (Promega, Cat: W6040) according to the manufacturer's instructions. The data was acquired using the GloMax® Discover GM3000 Microplate Reader (Promega).

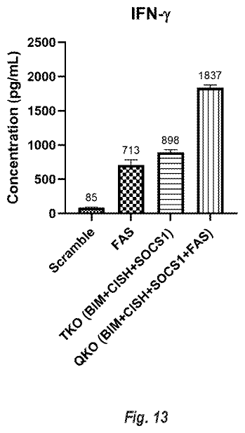

As shown in FIG. 13, the FAS^{-/-} SKO KRAS G12V TCR T-iαβT cells secreted higher amounts of IFN-g into the culture supernatant compared with control (scramble sgRNA-nucleofected) KRAS G12V TCR T-iαβT cells post co-culturing with GFP-HLA-A11-SW620-FASL cells at a 4:1 E:T ratio. There was also a significantly higher amount of IFN-g secretion in QKO (BIM^{-/-}/CISH^{-/-}/SOCS1^{-/-}FAS^{-/-}) compared with TKO (BIM^{-/-}/CISH^{-/-}/SOCS1^{-/-}) KRAS G12V TCR T-iαβT cells and FAS/SKO KRAS G12V TCR T-iαβT cells (FIG. 13), demonstrating synergistic effect in enhancing IFN-g release.

Overall, FAS deficiency protected KRAS G12V TCR T-iαβT cells from FASL-induced apoptosis and further increased IFN-g secretion post co-culturing with FASL-overexpressing HLA-A11-SW620 cells (FIG. 13).

Example 6. IFNγR-IL2/IL15R Signal Converter can Induce STAT Signaling, Increase Cell Expansion, Enhance Tumor Serial Killing in Mature iαβT 4KO Cells

### Example 6.1 Methods

### Virus Infection and Sorting

4KO (CISH^{-/-}/SOCS1^{-/-}/BIM^{-/-}/FAS^{-/-}) iPSCs were transduced with a lentiviral vector encoding KRAS G12V TCR (SEQ ID NOs: 79 and 80). 4KO (CISH^{-/-}/SOCS1^{-/-}/BIM^{-/-}/FAS^{-/-}) KRAS G12V TCR cells at the hematopoietic stem cell (HSC) stage (differentiated from 4KO KRAS G12V TCR (SEQ ID Nos: 79 and 80) T-iPSC) were co-transduced with lentiviral vectors encoding the IFNγR-IL2/IL15R signal converter, sequences of which are shown in Table 9. Following a 24-hour incubation period, the viral supernatant was replaced with fresh complete medium. The cells were subsequently sorted at the CD3⁺ lymphocyte stage to isolate those expressing the IFNγR-IL2/IL15R signal converter and then differentiated into mature iαβT cells. To evaluate the surface expression of the IFNγR-IL2/IL15R signal converter, the mature iαβT cells were stained with phycoerythrin (PE)-conjugated anti-human IFNγR1 antibody (1:100; BioLegend (Revvity, Waltham, MA), Cat. No. 308606) and allophycocyanin (APC)-conjugated anti-human IFNγR2 antibody (1:100; R&D Systems, Cat. No. FAB773A), in accordance with the manufacturers' instructions. Stained cells were analyzed using a flow cytometer, such as the Attune NxT Flow Cytometer (Thermo Fisher Scientific, Waltham, MA). The percentage of cells expressing the IFNγR-IL2/IL15R signal converter was quantified using FlowJo software (BD Biosciences, San Jose, CA).

### Western Blotting

Effector cells 4KO KRAS G12V TCR iαβT cells, without (Control cells) or with expressing IFNγR-IL2/IL15R signal converter (signal converter transduced cells) were subjected to cytokines starvation overnight. Then, effector cells were treated with 5 ng/mL IFN-γ in a time-dependent manner (15, 60, and 120 minutes), effector cells without IFN-γ treatment but with cytokines supplementation (iαβT cells were maintained in IL7 plus IL15 culture conditions) were set as positive control for both Control cells and signal converter transduced cells. Treated cells were harvested, and cell pellets were lysed in a radioimmunoprecipitation assay (RIPA) buffer (Millipore (Burlington, MA), Cat: 20-188) containing a protease inhibitor cocktail (TargetMol (Boston, MA), Cat: C0001) and phosphatase inhibitor cocktail I & II (Omics Bio, Cat: C0002 & C0003). The mixture was vortexed for 1 minute and put on ice for 30 minutes and then centrifuged at 12,000×g for 15 minutes at 4 °C. The supernatants were transferred to a new 1.6 mL microcentrifuge tube. The protein concentration of the cell lysate was determined by adding an acidic dye to a solution containing a protein, e.g., the Protein Assay Dye Reagent (Bio-Rad (Hercules, CA), Cat: 5000006). A total of 20 mg of protein in each lane was separated using a polyacrylamide gel, e.g. 4-15% Mini-PROTEAN® TGX™ Precast Protein Gels (Bio-Rad, Cat: 4561086). The separated proteins were subsequently transferred to a polyvinylidene fluoride (PVDF) membrane, e.g., a Power Blotter Select Transfer PVDF membrane (Invitrogen (Waltham, MA), Cat: PB5340). The transferred PVDF membrane was rinsed with 1×TBST (Tris buffered saline and 0.05% Tween 20) and blocked with 5% non-fat milk in TBST at room temperature for 1 hour. The blot was rinsed once for 10 minutes with TBST and then incubated with an anti-phospho-STAT1 (Tyr701) antibody (Cell Signaling Technology (Danvers, MA), Cat: 9167), an anti-phospho-STAT3 (Tyr705) antibody (Cell Signaling Technology, Cat: 9145), and an anti-phospho-STAT5 (Tyr694) antibody (Cell Signaling Technology, Cat: 9359) in 1% non-fat milk/TBST overnight at 4 °C. The blot was washed three times for 10 minutes with TBST and incubated with Peroxidase conjugated goat anti-rabbit IgG antibody (1:5,000; Sigma-Aldrich (St. Louis, MO), Cat: AP132P) in 1% non-fat milk/TBST at room temperature for 1 hour. The blot was washed three times for 10 minutes with TBST. A chemiluminescent (ECL) horseradish peroxidase (HRP) substrate, e.g. SuperSignal West Pico PLUS Chemiluminescent Substrate (ThermoFisher Scientific, Cat: 34580), was applied to the blot according to the manufacturer's recommendation. The chemiluminescent signals were captured using an imaging system, e.g. the ChemiDoc™ Imaging System (Bio-Rad). The blots were further stripped by Restore™ PLUS Western Blot Stripping Buffer (ThermoFisher Scientific, Cat: 46430) and re-probed with an anti-STAT1 antibody (Cell Signaling Technology, Cat: 14994), an anti-STAT3 antibody (Cell Signaling Technology, Cat: 9139), and an anti-STAT5 antibody (Cell Signaling Technology, Cat: 94205) in 1% non-fat milk/TBST overnight at 4 °C. Beta-actin (1:4,000; Santa Cruz Biotechnology (Dallas, TX), Cat: sc-47778 HRP) was used as an internal control to ensure almost equal amount of sample loading for each lane.

### Serial Killing Assay for GFP-HLA-A11-SW620 Cells

A total of 5×10³ GFP-HLA-A11-SW620 tumor cells were seeded into each well of a 96-well cell culture plate (Corning, Cat. No. 3599) and incubated overnight. Following incubation, the culture supernatant was aspirated, and effector cells—i.e., 4KO (CISH^{-/-}/SOCS1^{-/-}/BIM^{-/-}/FAS^{-/-}) KRAS G12V TCR iαβT cells—were added to each well in 200 μL of cytokine-free culture medium at an effector-to-target (E:T) cell ratio of 2:1. Tumor cell cytotoxicity was monitored using a live-cell imaging system, such as the Incucyte® SX5 Live-Cell Analysis System (Sartorius, Göttingen, Germany). To assess serial killing activity, effector cells were transferred to a new 96-well plate pre-seeded with GFP-HLA-A11-SW620 tumor cells after 48 hours of co-culture. The percentage of cytotoxicity was calculated based on EGFP fluorescence using the following formula:

Cytotoxicity percentage=[1−(Total EGFP fluorescence area at indicated timepoint/Total EGFP fluorescence area at the starting timepoint of each round of serial killing)]×100%

### Example 6.2 IFNγR-IL2/IL15R Signal Converter Design

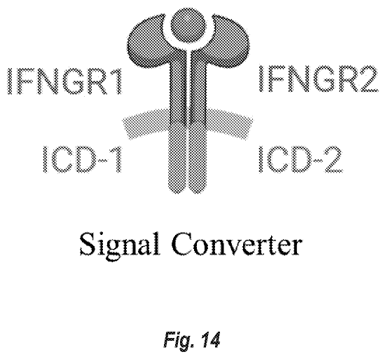

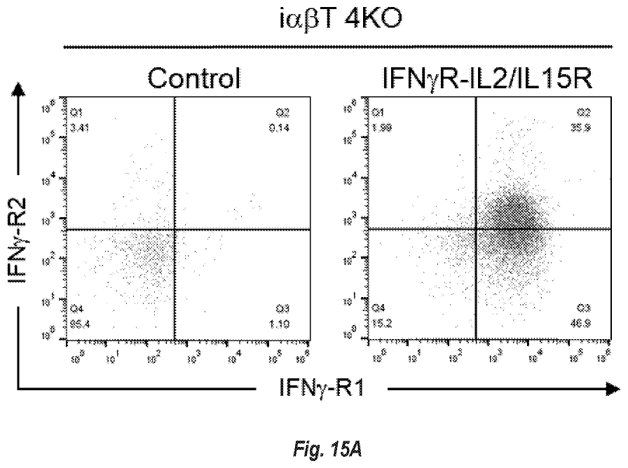

The IFNγR-IL2/IL15R signal converter (FIG. 14) contains a pair of subunits: one (IFNGR1 Subunit) has an IFNGR1 extracellular domain, while the other (IFNGR2 Subunit) has an IFNGR2 extracellular domain. IFNGR1 extracellular domain is fused sequentially with membrane-proximal region, transmembrane domain, and intracellular domain of common γ chain (γc) of IL2R (i.e., IL2RG, CD132). IFNGR2 extracellular domain is fused sequentially with membrane-proximal region, transmembrane domain, and intracellular domain of IL2RB. The structures and amino acid sequences of each IFNγR-IL2/IL15R signal converter subunits are provided in Table 9.

TABLE 9
Design and sequence table of IFNyR-IL2/IL15R signal converter

| Description | Sequence | SEQ ID NO: |
|---|---|---|
| ECD OF | EMGTADLGPSSVPTPTNVTIESYNMNPIVYWEYQIMPQVPVFTVEVKN | 63 |
| IFNGR1 | YGVKNSEWIDACINISHHYCNISDHVGDPSNSLWVRVKARVGQKESAY | |
| | AKSEEFAVCRDGKIGPPKLDIRKEEKQIMIDIFHPSVFVNGDEQEVDYDP | |
| | ETTCYIRVYNVYVRMNGSEIQYKILTQKEDDCDEIQCQLAIPVSSLNSQ | |
| | YCVSAEGVLHVWGVTTEKSKEVCITIFNSSIKG | |
| ECD of | APPDPLSQLPAPQHPKIRLYNAEQVLSWEPVALSNSTRPVVYQVQFKYT | 64 |
| IFNGR2 | DSKWFTADIMSIGVNCTQITATECDFTAASPSAGFPMDFNVTLRLRAEL | |
| | GALHSAWVTMPWFQHYRNVTVGPPENIEVTPGEGSLIIRFSSPFDIADT | |
| | STAFFCYYVHYWEKGGIQQVKGPFRSNSISLDNLKPSRVYCLQVQAQL | |
| | LWNKSNIFRVGHLSNISCYETMADASTELQQ | |
| Membrane- | FALEA | 65 |
| proximal region | | |
| of IL2RG | | |
| Membrane- | LGKDT | 66 |
| proximal region | | |
| of IL2RB | | |
| Transmembrane | VVISVGSMGLIISLLCVYFWL | 67 |
| domain of | | |
| IL2RG | | |
| Transmembrane | IPWLGHLLVGLSGAFGFIILVYLLI | 68 |
| domain of | | |
| IL2RB | | |
| Intracellular | ERTMPRIPTLKNLEDLVTEYHGNFSAWSGVSKGLAESLQPDYSERLCLV | 69 |
| domain of | SEIPPKGGALGEGPGASPCNQHSPYWAPPCYTLKPET | |
| IL2RG | | |
| Intracellular | NCRNTGPWLKKVLKCNTPDPSKFFSQLSSEHGGDVQKWLSSPFPSSSFS | 70 |
| domain of | PGGLAPEISPLEVLERDKVTQLLLQQDKVPEPASLSSNHSLTSCFTNQGY | |
| IL2RB | FFFHLPDALEIEACQVYFTYDPYSEEDPDEGVAGAPTGSSPQPLQPLSGE | |
| | DDAYCTFPSRDDLLLFSPSLLGGPSPPSTAPGGSGAGEERMPPSLQERVP | |
| | RDWDPQPLGPPTPGVPDLVDFQPPPELVLREAGEEVPDAGPREGVSFP | |
| | WSRPPGQGEFRALNARLPLNTDAYLSLQELQGQDPTHLV | |
| IFNGR1 | EMGTADLGPSSVPTPTNVTIESYNMNPIVYWEYQIMPQVPVFTVEVKN | 71 |
| Subunit: | YGVKNSEWIDACINISHHYCNISDHVGDPSNSLWVRVKARVGQKESAY | |
| IFNGR1- | AKSEEFAVCRDGKIGPPKLDIRKEEKQIMIDIFHPSVFVNGDEQEVDYDP | |
| IL2RG without | ETTCYIRVYNVYVRMNGSEIQYKILTQKEDDCDEIQCQLAIPVSSLNSQ | |
| signal peptide | YCVSAEGVLHVWGVTTEKSKEVCITIFNSSIKGFALEAVVISVGSMGLII | |
| | SLLCVYFWLERTMPRIPTLKNLEDLVTEYHGNFSAWSGVSKGLAESLQ | |
| | PDYSERLCLVSEIPPKGGALGEGPGASPCNQHSPYWAPPCYTLKPET | |
| IFNGR2 | APPDPLSQLPAPQHPKIRLYNAEQVLSWEPVALSNSTRPVVYQVQFKYT | 72 |
| Subunit: | DSKWFTADIMSIGVNCTQITATECDFTAASPSAGFPMDFNVTLRLRAEL | |
| IFNGR2- | GALHSAWVTMPWFQHYRNVTVGPPENIEVTPGEGSLIIRFSSPFDIADT | |
| IL2RB without | STAFFCYYVHYWEKGGIQQVKGPFRSNSISLDNLKPSRVYCLQVQAQL | |
| signal peptide | LWNKSNIFRVGHLSNISCYETMADASTELQQLGKDTIPWLGHLLVGLS | |
| | GAFGFIILVYLLINCRNTGPWLKKVLKCNTPDPSKFFSQLSSEHGGDVQ | |
| | KWLSSPFPSSSFSPGGLAPEISPLEVLERDKVTQLLLQQDKVPEPASLSS | |
| | NHSLTSCFTNQGYFFFHLPDALEIEACQVYFTYDPYSEEDPDEGVAGAP | |
| | TGSSPQPLQPLSGEDDAYCTFPSRDDLLLFSPSLLGGPSPPSTAPGGSGA | |
| | GEERMPPSLQERVPRDWDPQPLGPPTPGVPDLVDFQPPPELVLREAGEE | |
| | VPDAGPREGVSFPWSRPPGQGEFRALNARLPLNTDAYLSLQELQGQDP | |
| | THLV | |
| Signal peptide | MALLFLLPLVMQGVSRA | 73 |
| of IFNGR1 | | |
| Signal peptide | MRPTLLWSLLLLLGVFAAAAA | 74 |
| of IFNGR2 | | |
| IFNGR1 | MALLFLLPLVMQGVSRAEMGTADLGPSSVPTPTNVTIESYNMNPIVYW | 75 |
| Subunit: | EYQIMPQVPVFTVEVKNYGVKNSEWIDACINISHHYCNISDHVGDPSN | |
| IFNGR1- | SLWVRVKARVGQKESAYAKSEEFAVCRDGKIGPPKLDIRKEEKQIMIDIF | |
| IL2RG with | HPSVFVNGDEQEVDYDPETTCYIRVYNVYVRMNGSEIQYKILTQKEDD | |
| signal peptide | CDEIQCQLAIPVSSLNSQYCVSAEGVLHVWGVTTEKSKEVCITIFNSSIK | |
| | GFALEAVVISVGSMGLIISLLCVYFWLERTMPRIPTLKNLEDLVTEYHGN | |
| | FSAWSGVSKGLAESLQPDYSERLCLVSEIPPKGGALGEGPGASPCNQHS | |
| | PYWAPPCYTLKPET | |
| IFNGR2 | MRPTLLWSLLLLLGVFAAAAAAPPDPLSQLPAPQHPKIRLYNAEQVLS | 76 |
| Subunit: | WEPVALSNSTRPVVYQVQFKYTDSKWFTADIMSIGVNCTQITATECDFT | |
| IFNGR2- | AASPSAGFPMDFNVTLRLRAELGALHSAWVTMPWFQHYRNVTVGPPE | |
| IL2RB with | NIEVTPGEGSLIIRFSSPFDIADTSTAFFCYYVHYWEKGGIQQVKGPFRS | |
| signal peptide | NSISLDNLKPSRVYCLQVQAQLLWNKSNIFRVGHLSNISCYETMADAST | |
| | ELQQLGKDTIPWLGHLLVGLSGAFGFIILVYLLINCRNTGPWLKKVLKC | |
| | NTPDPSKFFSQLSSEHGGDVQKWLSSPFPSSSFSPGGLAPEISPLEVLER | |
| | DKVTQLLLQQDKVPEPASLSSNHSLTSCFTNQGYFFFHLPDALEIEACQ | |
| | VYFTYDPYSEEDPDEGVAGAPTGSSPQPLQPLSGEDDAYCTFPSRDDLL | |
| | LFSPSLLGGPSPPSTAPGGSGAGEERMPPSLQERVPRDWDPQPLGPPTPG | |
| | VPDLVDFQPPPELVLREAGEEVPDAGPREGVSFPWSRPPGQGEFRALNA | |
| | RLPLNTDAYLSLQELQGQDPTHLV | |

Example 6.3 Validation of the Functions of the IFNγR-IL2/IL15R Signal Converter in iPSC-Derived Mature iαβT 4KO KRAS G12V TCR Cells

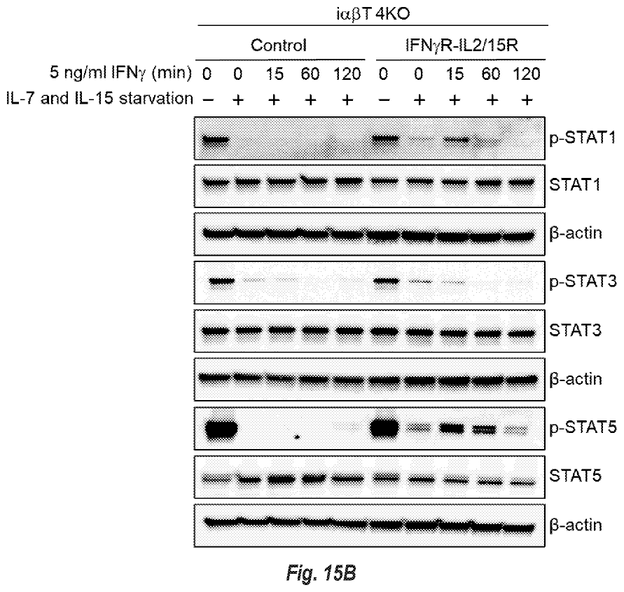

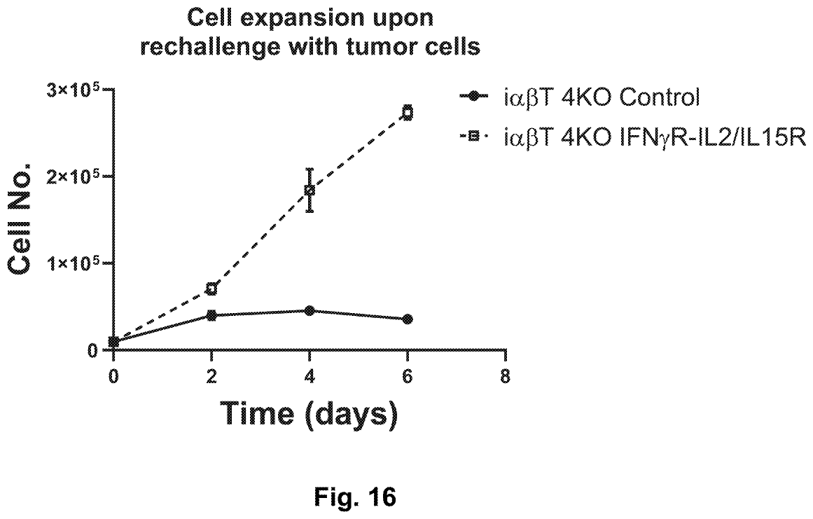

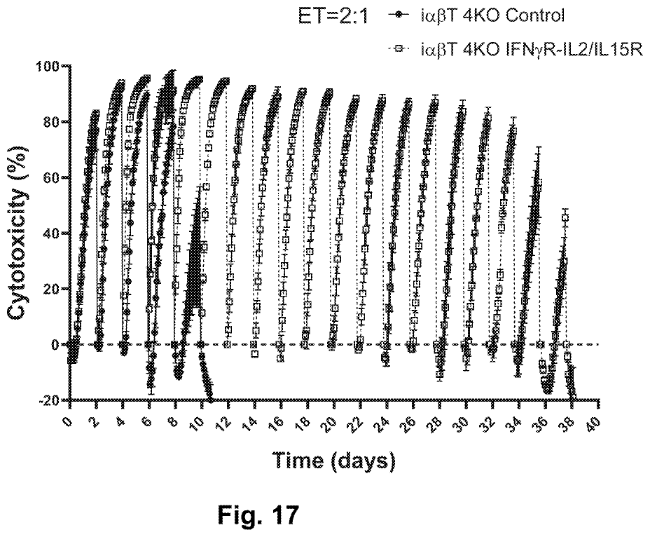

To evaluate the functional activity of the IFNγR-IL2/IL15R signal converter in induced pluripotent stem cell (iPSC)-derived mature 4KO (CISH^{-/-}/SOCS1^{-/-}/BIM^{-/-}/FAS^{-/-}) KRAS G12V TCR iαβT cells, nucleic acids encoding the signal converter were introduced via viral transduction, followed by cell sorting as described in Example 6.1. As shown in FIG. 15A, the percentage of cells with IFNγR-IL2/IL15R signal converter in mature 4KO KRAS G12V TCR iαβT cells was approximately 35.9%. Upon IFN-γ stimulation, phosphorylation of STAT1 and STAT5 was markedly increased in mature 4KO KRAS G12V TCR iαβT cells with IFNγR-IL2/IL15R signal converter, as demonstrated in FIG. 15B. A cell expansion assay was subsequently performed under conditions of repeated tumor antigen stimulation. As illustrated in FIG. 16, mature 4KO KRAS G12V TCR iαβT cells expressing the IFNγR-IL2/IL15R signal converter exhibited approximately 7.5-fold greater expansion over a 6-day period compared to control 4KO KRAS G12V TCR iαβT cells lacking the signal converter. Furthermore, as shown in FIG. 17, the IFNγR-IL2/IL15R signal converter substantially enhanced the serial tumor killing activity of mature iαβT 4KO KRAS G12V TCR cells against GFP-HLA-A11-SW620 tumor cells and maintained serial tumor killing effect for around 18 rounds at a 2:1 effector to target cell ratio, compared with around 5 rounds when lacking IFNγR-IL2/IL15R signal converter.

Collectively, these results demonstrate that overexpression of the IFNγR-IL2/IL15R signal converter in iPSC-derived mature iαβT 4KO KRAS G12V TCR cells induces STAT1 and STAT5 activation, promotes robust cell expansion, and significantly enhances multi-round tumor killing capacity.

Example 7. Functionality of HSV-TK/GCV in iPSC and iPSC-Derived abT Cells.

This is to evaluate the functional activity of delta-HSV (herpes simplex virus)-TK (thymidine kinase)-A168H in B2M^{-/-}/CIITA^{-/-}/B2M-HLA-E knock-in induced pluripotent stem cell (iPSC) and iPSC-derived mature iabT cells. The amino acid sequence of the delta-HSV-TK-A168H is shown as SEQ ID NO: 85 in Table 11. The amino acid sequences of P2A and B2M-HLA-E-P2A-delta-HSV-TK-A168H are shown as SEQ ID NO: 84 and 86 in Table12. The P2A peptide is a short self-cleaving sequence, allowing multiple proteins to be co-expressed from a single transcript, and the P2A sequence itself is ultimately cleaved off so that the upstream and downstream proteins are produced as separate polypeptides.

To generate B2M-HLA-E-P2A-delta-HSV-TK-A168H (SEQ ID NO: 86 in Table11) gene knock-in in CIITA^{-/-} αβT-derived iPSC cells, 5×10⁶ of CIITA^{-/-} αβT-derived iPSC cells were nucleofected with 200 μmol of Alt-R™ S.p. HiFi Cas9 Nuclease V3 (Integrated DNA Technologies (Coralville, IA); Cat: 1081060) and synthetic β2M single guide RNA (sgRNA) (β2M #1 in Table 10) mixed with B2M-HLA-E-P2A-delta-HSV-TK-A168H donor plasmid by Lonza P3 Primary Cell 4D-Nucleofector® X Kit L (Lonza (Basel, Switzerland), Cat: V4XP-3024) and Lonza 4D Nucleofector® Core and X Unit (Lonza, Cat: AAF-1003X) per Nucleocuvette vessel. Nucleofected iPSC cells were seeding in VTN coated 96 well plate for cell sorting. B2M-HLA-E is used to enrich B2M-HLA-E-P2A-delta-HSV-TK-A168H knock-in population.

After cell sorting and enrichment, the resulted B2M^{-/-}/CIITA^{-/-}/B2M-HLA-E-P2A-delta-HSV-TK-A168H knock-in iPSC cells were seeded into a VTN coated 96 well plate for cell viability assay with Ganciclovir (GCV) (Calbiochem, Cat: 345700) treatment. After seeding overnight, the cells were treated with Ganciclovir (GCV) at dosage ranges from 10-4 to 10 μM in triplicate for 48 hours. CellTiter-Glo 2.0 luminescent luciferase assay reagents (Promega, Cat: G9241) were used for cell viability assay. The cell viability was analyzed by the GloMax® Discover GM3000 Microplate Reader (Promega).

The cell viability percentage was calculated by the formula listed below:

Cell viability percentage=[the luminescence (RLU) of GCV treated cells/the average luminescence of untreated cells]×100%

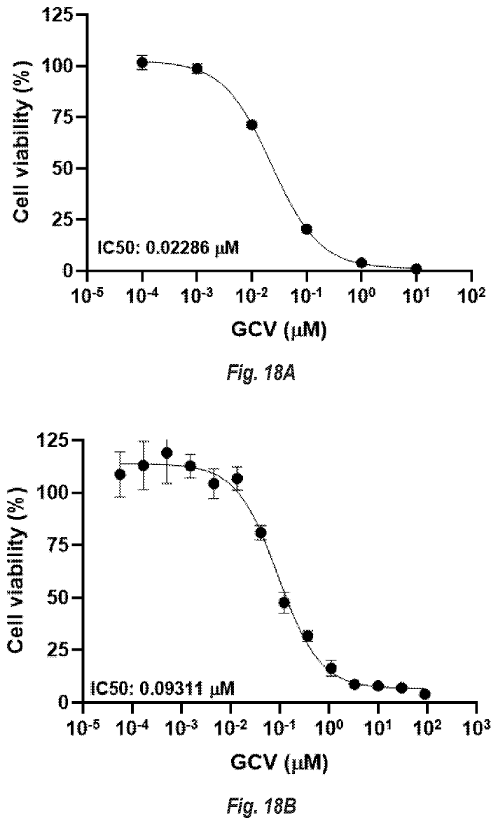

The IC50 of cell viability with B2M^{-/-}/CIITA^{-/-}/B2M-HLA-E-P2A-delta-HSV-TK-A168H iPSC was approximately 0.02286 μM. Upon GCV treatment, incorporation of GCV into DNA following its metabolization by thymidine kinase induced cell cycle arrest. As the GCV concentration increased, this effect became more pronounced and progressively reduced cell viability. As shown in FIG. 18A, the GCV induced dose dependent cell death in the presence of delta-HSV-TK-A168H, demonstrating that GCV could work as a kill switch for B2M^{-/-}/CIITA^{-/-}/B2M-HLA-E-P2A-delta-HSV-TK-A168H iPSC.

B2M^{-/-}/CIITA^{-/-}/B2M-HLA-E-P2A-delta-HSV-TK-A168H iPSC were undergone differentiation in to the iPSC-derived iαβT, followed by cell viability assay with Ganciclovir (GCV) treatment.

The IC50 of cell viability with B2M^{-/-}/CIITA^{-/-}/B2M-HLA-E-P2A-delta-HSV-TK-A168H iαβT was approximately 0.09311 μM. As shown in FIG. 18B, the GCV induced dose-dependent cell death in the presence of delta-HSV-TK-A168H, demonstrating that GCV could work as a kill switch for B2M^{-/-}/CIITA^{-/-}/B2M-HLA-E-P2A-delta-HSV-TK-A168H iαβT.

TABLE 10
sgRNA sequence of B2M and CIITA used for nucleofection with Cas9 nuclease

| | Name | | Targeting/guide | Target | |
|---|---|---|---|---|---|
| Target | of | | sequence in | sequence in | Target |
| Gene | sgRNA | Synthetic sgRNA sequence | sgRNA | target gene | exon |
| B2M | B2M | mC*mG*mU*rGrArGrUrArAr | CGTGAGTAAAC | AAGATTCAG | Exon 2 |
| | sgRNA | ArCrCrUrGrArArUrCrUrUrGr | CTGAATCTT | GTTTACTCAC | |
| | (B2M | UrUrUrUrArGrArGrCrUrArGr | (SEQ ID NO: 55) | G | |
| | #1) | ArArArUrArGrCrArArGrUrUr | | (SEQ ID NO: | |
| | | ArArArArUrArArGrGrCrUrAr | | 56) | |
| | | GrUrCrCrGrUrUrArUrCrArArC | | | |
| | | rUrUrGrArArArArArGrUrGrGr | | | |
| | | CrArCrCrGrArGrUrCrGrGrUrG | | | |
| | | rCmU*mU*mU*rU (SEQ ID NO: | | | |
| | | 54) | | | |
| CIITA | CIITA | mG*mA*mU*rArUrUrGrGrCr | GATATTGGCAT | GGGAGGCTT | Exon 3 |
| | sgRNA | ArUrArArGrCrCrUrCrCrCrGrU | AAGCCTCCC | ATGCCAATAT | |
| | (CIITA | rUrUrUrArGrArGrCrUrArGrAr | (SEQ ID NO: 58) | C | |
| | #1) | ArArUrArGrCrArArGrUrUrAr | | (SEQ ID NO: | |
| | | ArArArUrArArGrGrCrUrArGr | | 59) | |
| | | UrCrCrGrUrUrArUrCrArArCrU | | | |
| | | rUrGr ArArArArArGrUrGrGrCr | | | |
| | | ArCrCrGrArGrUrCrGrGrUrGrC | | | |
| | | mU*mU*mU*rU (SEQ ID NO: | | | |
| | | 57) | | | |

TABLE 11
Sequences of delta-HSV-TK-A168H

| | | |
|---|---|---|
| SEQ ID | B2M-HLA-E | MSRSVALAVLALLSLSGLEAVMAPRTLILGGGGSGGGGSGGGGSIQRTPKIQ |
| NO: 81 | fusion protein | VYSRHPAENGKSNFLNCYVSGFHPSDIEVDLLKNGERIEKVEHSDLSFSKD |
| | | WSFYLLYYTEFTPTEKDEYACRVNHVTLSQPKIVKWDRDMGGGGSGGGGS |
| | | GGGGSGGGGSGSHSLKYFHTSVSRPGRGEPRFISVGYVDDTQFVRFDNDAA |
| | | SPRMVPRAPWMEQEGSEYWDRETRSARDTAQIFRVNLRTLRGYYNQSEAG |
| | | SHTLQWMHGCELGPDGRFLRGYEQFAYDGKDYLTLNEDLRSWTAVDTAAQ |
| | | ISEQKSNDASEAEHQRAYLEDTCVEWLHKYLEKGKETLLHLEPPKTHVTHH |
| | | PISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGS |
| | | VVSGAVVAAVIWRKKSSGGKGGSYSKAEWSDSAQGSESHSL* |
| SEQ ID | P2A | GSGATNFSLLKQAGDVEENPGP |
| NO: 84 | | |
| SEQ ID | delta-HSV- | MPTLLRVYIDGPHGMGKTTTTQLLVALGSRDDIVYVPEPMTYWQVLGASET |
| NO: 85 | TK-A168H | IANIYTTQHRLDQGEISAGDAAVVMTSAQITMGMPYAVTDAVLAPHIGGEA |
| | | GSSHAPPPALTLIFDRHPIAHLLCYPAARYLMGSMTPQAVLAFVALIPPTLPG |
| | | TNIVLGALPEDRHIDRLAKRQRPGERLDLAMLAAIRRVYGLLANTVRYLQG |
| | | GGSWREDWGQLSGTAVPPQGAEPQSNAGPRPHIGDTLFTLFRAPELLAPNG |
| | | DLYNVFAWALDVLAKRLRPMHVFILDYDQSPAGCRDALLQLTSGMVQTHV |
| | | TTPGSIPTICDLARTFAREMGEAN |
| SEQ ID | B2M-HLA- | MSRSVALAVLALLSLSGLEAVMAPRTLILGGGGSGGGGSGGGGSIQRTPKIQ |
| NO: 86 | E-P2A-delta- | VYSRHPAENGKSNFLNCYVSGFHPSDIEVDLLKNGERIEKVEHSDLSFSKD |
| | HSV-TK- | WSFYLLYYTEFTPTEKDEYACRVNHVTLSQPKIVKWDRDMGGGGSGGGGS |
| | A168H | GGGGSGGGGSGSHSLKYFHTSVSRPGRGEPRFISVGYVDDTQFVRFDNDAA |
| | | SPRMVPRAPWMEQEGSEYWDRETRSARDTAQIFRVNLRTLRGYYNQSEAG |
| | | SHTLQWMHGCELGPDGRFLRGYEQFAYDGKDYLTLNEDLRSWTAVDTAAQ |
| | | ISEQKSNDASEAEHQRAYLEDTCVEWLHKYLEKGKETLLHLEPPKTHVTHH |
| | | PISDHEATLRCWALGFYPAEITLTWQQDGEGHTQDTELVETRPAGDGTFQK |
| | | WAAVVVPSGEEQRYTCHVQHEGLPEPVTLRWKPASQPTIPIVGIIAGLVLLGS |
| | | VVSGAVVAAVIWRKKSSGGKGGSYSKAEWSDSAQGSESHSLGSGATNFSLL |
| | | KQAGDVEENPGPMPTLLRVYIDGPHGMGKTTTTQLLVALGSRDDIVYVPEP |
| | | MTYWQVLGASETIANIYTTQHRLDQGEISAGDAAVVMTSAQITMGMPYAV |
| | | TDAVLAPHIGGEAGSSHAPPPALTLIFDRHPIAHLLCYPAARYLMGSMTPQA |
| | | VLAFVALIPPTLPGTNIVLGALPEDRHIDRLAKRQRPGERLDLAMLAAIRRV |
| | | YGLLANTVRYLQGGGSWREDWGQLSGTAVPPQGAEPQSNAGPRPHIGDTL |
| | | FTLFRAPELLAPNGDLYNVFAWALDVLAKRLRPMHVFILDYDQSPAGCRDA |
| | | LLQLTSGMVQTHVTTPGSIPTICDLARTFAREMGEAN |

Example 8. IFNγR-IL2/IL15R Signal Converter can Enhance Tumor Cell Serial Killing in Mature CD19 CAR iαβT 4KO Cells

### Example 8.1 Methods

### Virus Infection and Sorting

4KO (CISH^{-/-}/SOCS1^{-/-}/BIM^{-/-}/FAS^{-/-}) iPSCs were transduced with a lentiviral vector encoding KRAS G12V TCR (SEQ ID NOs: 79 and 80). KRAS G12V TCR positive iPSCs were enriched by cell sorting (FACSAria™ III) following staining with an anti-TCR antibody (1:100; Biolegend, Cat. No. 109212). The 4KO KRAS G12V TCR iPSCs were then differentiated into 4KO KRAS G12V TCR hematopoietic stem cells (iHSCs) and the iHSCs were co-transduced with lentiviral vectors encoding the IFNγR-IL2/IL15R signal converter (sequences are shown in Table 9) and CD19 CAR (SEQ ID NO: 94). The method is similar to the method shown in Example 6.1. To evaluate the surface expression of CD19 CAR, the mature iαβT cells were stained with Alexa Fluor 647-conjugated anti-FMC63 antibody (1:100; Acro Biosystem, Cat. No. FM3-AM534).

### Flow Cytometry-Based Quantification of T Cell Expansion Following Serial Tumor Cell Stimulation

A total of 5×10³ Nalm6-iRFP713 tumor cells (endogenously expressing CD19) and indicated effector cells (e.g., 4KO KRAS G12V TCR iαβT expressing CD19 CAR and IFNγR-IL2/IL15R) were co-cultured at designated effector-to-target (E:T) ratio of 4:1 per well in 96-well culture plate (Corning, Cat. No. 3599) and incubated at 37 °C. The expansion of effector cells was assessed under daily Nalm6-iRFP713 tumor cell rechallenge performed in cytokine-free assay medium. At each desired time point, co-cultured cells from the relevant wells were transferred into a 96-well U-bottom plate for staining. Cells were centrifuged at 500×g for 3 minutes at room temperature (20-25 °C.), and the supernatant was discarded. The cell pellet was washed once with flow staining buffer (2% heat-inactivated fetal bovine serum (FBS) in autoMACS® Rinsing Solution; Miltenyi Biotec, Cat. No. 130-091-222). Cells were incubated with anti-human CD3 antibody (1:200; Miltenyi Biotec, Cat. No. 130-113-141) in flow staining buffer for 20 minutes at room temperature, protected from light. After incubation, cells were centrifuged again at 500×g for 3 minutes at room temperature, and the supernatant was discarded. The cell pellet was washed once more with flow staining buffer. Cells were then resuspended in 200 μL of flow staining buffer containing Propidium Iodide (PI, 1:2500; BioLegend, Cat. No. 421301) to identify dead cells, and Precision Count Beads (1:100; BioLegend, Cat. No. 424902) to enable absolute quantification. Samples were acquired on a flow cytometer, and data were analyzed using FlowJo software. The absolute number of viable CD3+ T cells was calculated based on bead-based quantification using the following formula:

Absolute Cell Count(Cells/μL)=[Cell Count×Precision Count Beads™ Volume/Precision Count Beads™ Count×Cell Volume]×Precision Count Beads™ Concentration(Beads/μL)

### Preparation of Primary αβT Expressing CD19 CAR

Human primary CD3 T cells were enriched from PBMC by pan T cell isolation kit (Miltenyi Biotec, Cat. No. 130-096-535). 1E6 of isolated T cells were activated by incubation with 25 μL of human T-activator CD3/CD28 beads (Gibco, Cat. No. 11132D) in 1 mL RPMI-1640 (Gibco, Cat. No. A1049101) supplemented with 10% heat-inactivated FBS (Gibco, Cat. No. 52567-01), 10 ng/mL human IL-7 and 10 ng/mL human IL-15 for 2 days. Cells were then transduced with CD19 CAR lentivirus in the presence of polybrene at 400×g via spin transduction for 1 hour, followed by culture for additional 2 days. On day 4, the cells were separated from beads and maintained at a cell density of 1E6/mL for 5 days in the medium with IL-7 and IL-15. The cells expressing CD19 CAR were collected at day 9 for evaluating CAR expression by flow cytometry and for functional assay.

### Serial Tumor Cell Killing Assay

A total of 5×10³ Nalm-6-iRFP713 tumor cells were seeded into each well of a 96-well cell culture plate (Corning, Cat. No. 3599). The effector cells, e.g., 4KO (CISH^{-/-}/SOCS1^{-/-}/BIM^{-/-}/FAS^{-/-}) KRAS G12V TCR iαβT cells expressing CD19 CAR and IFNγR-IL2/IL15R or primary αβT cells expressing CD19 CAR, were added to each well in 200 μL of cytokine-free culture medium at an effector-to-target (E:T) cell ratio of 4:1. Tumor cell cytotoxicity was monitored using a live-cell imaging system, such as the Incucyte® SX5 Live-Cell Analysis System (Sartorius, Göttingen, Germany). To assess serial killing activity, Nalm-6-iRFP713 tumor cells were added to an original 96-well plate after 24 hours of co-culture, and were supplemented every 24 hours. The percentage of cytotoxicity was calculated based on iRFP fluorescence using the following formula:

Cytotoxicity percentage=[1−(Total iRFP fluorescence area at indicated timepoint/Total iRFP fluorescence area at the starting timepoint of each round of serial killing)]×100%

### Cytokine Release Assay (Bead-Based Multiplex Analysis)

At the endpoint of the tumor cell killing assay, half of the co-culture supernatant from the target-effector mixture was collected to measure cytokine production. Cytokine release assays were performed using the bead-based LEGENDplex™ system (BioLegend, Cat No. 741186) according to the manufacturer's protocols.

TABLE 12
Design and sequence table of CD19-CAR

| SEQ ID | CD8a signal | |
|---|---|---|
| NO: 87 | peptide | MALPVTALLLPLALLLHAARP |
| SEQ ID | CD19 scFv | DIQMTQTTSSLSASLGDRVTISCRASQDISKYLNWYQQKPDGTVKLLIYHTSR |
| NO: 88 | | LHSGVPSRFSGSGSGTDYSLTISNLEQEDIATYFCQQGNTLPYTFGGGTKLEIT |
| | | GGGGSGGGGSGGGGSEVKLQESGPGLVAPSQSLSVTCTVSGVSLPDYGVSWI |
| | | RQPPRKGLEWLGVIWGSETTYYNSALKSRLTIIKDNSKSQVFLKMNSLQTDD |
| | | TAIYYCAKHYYYGGSYAMDYWGQGTSVTVSS |
| SEQ ID | CD8a loop | TTTPAPRPPTPAPTIASQPLSLRPEACRPAAGGAVHTRGLDFACD |
| NO: 89 | | |
| SEQ ID | CD8a TM | IYIWAPLAGTCGVLLLSLVITLYC |
| NO: 90 | | |
| SEQ ID | 41BB ICD | KRGRKKLLYIFKQPFMRPVQTTQEEDGCSCRFPEEEEGGCEL |
| NO: 91 | | |
| SEQ ID | CD3z AD | RVKFSRSADAPAYKQGQNQLYNELNLGRREEYDVLDKRRGRDPEMGGKPRR |
| NO: 92 | | KNPQEGLYNELQKDKMAEAYSEIGMKGERRRGKGHDGLYQGLSTATKDTYD |
| | | ALHMQALPPR |
| SEQ ID | CD19-CAR | DIQMTQTTSSLSASLGDRVTISCRASQDISKYLNWYQQKPDGTVKLLIYHTSR |
| NO: 93 | without | LHSGVPSRFSGSGSGTDYSLTISNLEQEDIATYFCQQGNTLPYTFGGGTKLEIT |
| | signal | GGGGSGGGGGGGGSEVKLQESGPGLVAPSQSLSVTCTVSGVSLPDYGVSWI |
| | peptide | RQPPRKGLEWLGVIWGSETTYYNSALKSRLTIIKDNSKSQVFLKMNSLQTDD |
| | | TAIYYCAKHYYYGGSYAMDYWGQGTSVTVSSTTTPAPRPPTPAPTIASQPLSL |
| | | RPEACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| SEQ ID | CD19-CAR | MALPVTALLLPLALLLHAARPDIQMTQTTSSLSASLGDRVTISCRASQDISKYL |
| NO: 94 | with signal | NWYQQKPDGTVKLLIYHTSRLHSGVPSRFSGSGSGTDYSLTISNLEQEDIATY |
| | peptide | FCQQGNTLPYTFGGGTKLEITGGGGSGGGGSGGGGSEVKLQESGPGLVAPSQ |
| | | SLSVTCTVSGVSLPDYGVSWIRQPPRKGLEWLGVIWGSETTYYNSALKSRLTI |
| | | IKDNSKSQVFLKMNSLQTDDTAIYYCAKHYYYGGSYAMDYWGQGTSVTVSS |
| | | TTTPAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEGG |
| | | CELRVKFSRSADAPAYKQGQNQLYNELNLGRREEYDVLDKRRGRDPEMGGK |
| | | PRRKNPQEGLYNELQKDKMAEAYSEIGMKGERRRGKGHDGLYQGLSTATKD |
| | | TYDALHMQALPPR |

Example 8.2 Validation of the Functions of the IFNγR-IL2/IL15R Signal Converter in iPSC-Derived Mature 4KO KRAS G12V TCR iαβT Cells Expressing CD19 CAR and IFNγR-IL2/IL15R

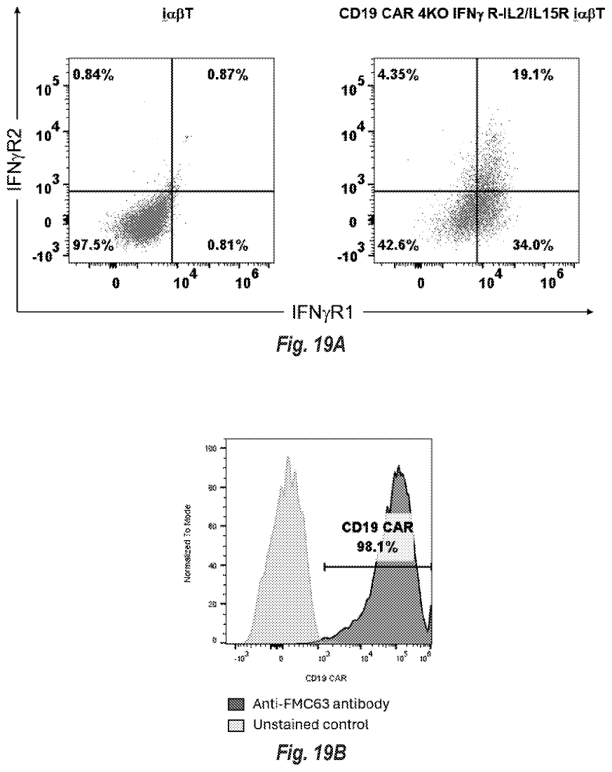

To evaluate the functional activity of the IFNγR-IL2/IL15R signal converter in induced pluripotent stem cell (iPSC)-derived mature 4KO (CISH^{-/-}/SOCS1^{-/-}/BIM^{-/-}/FAS^{-/-}) KRAS G12V TCR iαβT cells expressing CD19 CAR and IFNγR-IL2/IL15R, nucleic acids encoding the signal converter and CD19 CAR were introduced via viral transduction, followed by cell sorting as described in Example 8.1. As shown in FIGS. 19A and 19B, the percentages of 4KO (CISH^{-/-}/SOCS1^{-/-}/BIM^{-/-}/FAS^{-/-}) KRAS G12V TCR iαβT cells expressing IFNγR-IL2/IL15R or CD19 CAR were approximately 19.1% and 98%, respectively. 4KO (CISH^{-/-}/SOCS1^{-/-}/BIM^{-/-}/FAS^{-/-}) KRAS G12V TCR iαβT cells expressing CD19 CAR and IFNγR-IL2/IL15R (hereafter referred to “4KO iαβT cells expressing CD19 CAR and IFNγR-IL2/IL15R”) were used for further evaluation.

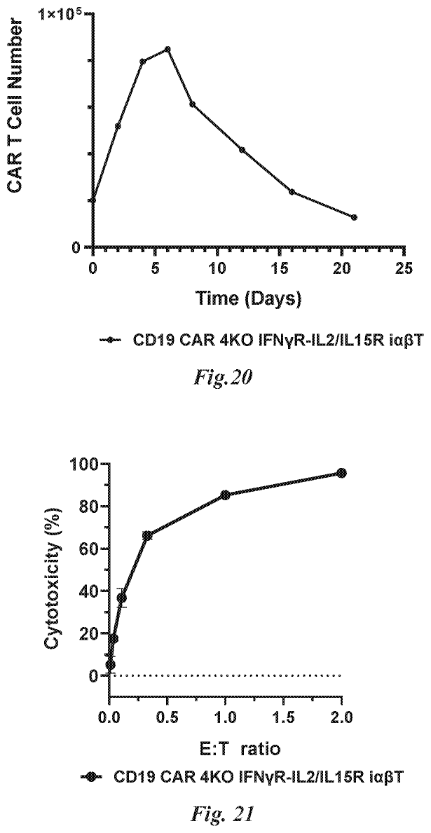

A cell expansion assay was performed with repeated Nalm-6-iRFP713 tumor cell rechallenge. At day 0, 2, 4, 6, 8, 12, 16 and 21, 4KO iαβT cells expressing CD19 CAR and IFNγR-IL2/IL15R were counted. As illustrated in FIG. 20, 4KO iαβT cells expressing CD19 CAR and IFNγR-IL2/IL15R exhibited approximately 4.2-fold expansion over a 6-day period.

To evaluate cell potency, different effector-to-target (E:T) ratios were tested in a single-run tumor cell killing assay. As shown in FIG. 21, 4KO iαβT cells expressing CD19 CAR and IFNγR-IL2/IL15R were co-cultured with Nalm-6-iRFP713 at defined E:T ratio (0.01, 0.03, 0.1, 0.3, 1 or 2) for 24 hours. 4KO iαβT cells expressing CD19 CAR and IFNγR-IL2/IL15R showed dose-dependent tumor killing potency and substantial tumor elimination at E:T ratio of 1 or 2.

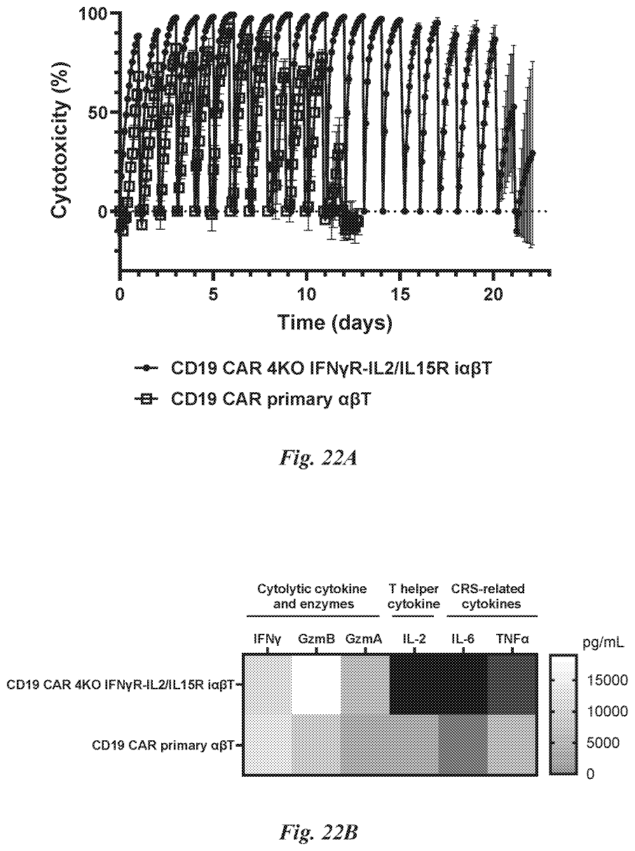

Furthermore, serial tumor cell killing assay were conducted using 4KO iαβT cells expressing CD19 CAR and IFNγR-IL2/IL15R and primary αβT expressing CD19 CAR. As shown in FIG. 22A, 4KO iαβT cells expressing CD19 CAR and IFNγR-IL2/IL15R showed persistent tumor killing activity against Nalm-6-iRFP713 tumor cells at a 4:1 E:T ratio, maintaining the serial tumor killing for around 21 rounds, compared with around 11 rounds killing of primary αβT cells expressing CD19 CAR.

Cytokine secretion assay was also performed. As shown in FIG. 22B, cytokine release syndrome (CRS)-related cytokines, including IL-6 and TNF-α, were at non-detectable levels for 4KO iαβT cells expressing CD19 CAR and IFNγR-IL2/IL15R, compared with those for primary αβT cells expressing CD19 CAR. In addition, 4KO iαβT cells expressing CD19 CAR and IFNγR-IL2/IL15R also secreted IFNγ and effector enzymes (i.e., Granzyme A and Granzyme B) to support T-cell function. Notably, no IL-2 secretion was detected in 4KO iαβT cells expressing CD19 CAR and IFNγR-IL2/IL15R, indicating the cell can sustain cell expansion without IL-2 support, offering improved functional durability for cell therapy development.

Collectively, these results demonstrate that 4KO iαβT cells expressing CD19 CAR and IFNγR-IL2/IL15R exhibit great cell expansion and superior persistent tumor cell killing, while not inducing CRS-related cytokine release or IL-2 secretion.

1-50. (canceled)

51. A modified αβT cell, comprising 1) an exogenous polynucleotide encoding a chimeric antigen receptor (CAR) which specifically binds human CD19 and/or an exogenous polynucleotide encoding a chimeric antigen receptor (CAR) which specifically binds human CD20; 2) a genomic disruption in an endogenous suppressor of cytokine signaling 1 (SOCS1) gene that suppresses or eliminates expression of a functional SOCS1 protein; 3) a genomic disruption in an endogenous cytokine-inducible SH2-containing protein (CISH) gene that suppresses or eliminates expression of a functional CISH protein; and 4) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell.

52. The modified cell of claim 51, wherein the genomic disruption in the endogenous SOCS1 gene is in exon 2 of the SOCS1 gene; and/or wherein the genomic disruption in the endogenous CISH gene is in exon 3 or exon 4 of the CISH gene.

53. The modified cell of claim 51,
(i) wherein the genomic disruption in the endogenous BIM gene is within a target sequence in BIM that comprises any one of SEQ ID NOs: 14, 20, 21, and 22;
(ii) wherein the genomic disruption in the endogenous SOCS1 gene is within a target sequence in SOCS1 that comprises SEQ ID NO: 17 or SEQ ID NO: 23; and
(iii) wherein the genomic disruption in the endogenous CISH gene is within a target sequence in CISH that comprises SEQ ID NO: 15, SEQ ID NO: 16 or SEQ ID NO: 24.

54. The modified cell of claim 51, wherein the genomic disruption is made by administering one or more guide RNAs to said cell, and wherein an endonuclease or a nucleic acid encoding the endonuclease is also administered to said cell; and wherein the genomic disruption is an inactivating nucleic acid mutation, optionally wherein the mutation is an insertion, deletion, or substitution.

55. The modified cell of claim 54,
(i) wherein (1) the endonuclease is the MAD7 endonuclease, and the guide RNAs comprise the guide sequence of SEQ ID NO: 11, or comprise the crRNA sequence of SEQ ID NO: 37; or (2) the endonuclease is the Cas9 endonuclease, and the guide RNAs comprise the guide sequence of SEQ ID NO: 5, or comprise the sgRNA sequence of SEQ ID NO: 29;
(ii) wherein (1) the endonuclease is the MAD7 endonuclease, and the guide RNAs comprise the guide sequence of SEQ ID NO: 12, or comprise the crRNA sequence of SEQ ID NO: 38; or (2) the endonuclease is the Cas9 endonuclease, and the guide RNAs comprise the guide sequence of any one of SEQ ID NOs: 3 and 4, or comprise the sgRNA sequence of any one of SEQ ID NOs: 27 and 28; and
(iii) wherein (1) the endonuclease is the MAD7 endonuclease, and the guide RNAs comprise the guide sequence of SEQ ID NO: 10, or comprise the crRNA sequence of SEQ ID NO: 36; or (2) the endonuclease is the Cas9 endonuclease, and the guide RNAs comprise the guide sequence of any one of SEQ ID NOs: 2, 8, and 9, or comprise the sgRNA sequence of any one of SEQ ID NOs: 26, 34, and 35.

56. The modified cell of claim 51, wherein the exogenous polynucleotide encoding the CAR which specifically binds human CD19 is inserted into an AAVS1 locus; and wherein the CAR which specifically binds human CD19 comprises the amino acid sequence of SEQ ID NO: 93 or SEQ ID NO: 94.

57. The modified cell of claim 51, further comprising a genomic disruption in an endogenous FAS cell surface death receptor (FAS) gene that suppresses or eliminates expression of a functional FAS protein.

58. The modified cell of claim 51, further comprising a genomic disruption in an endogenous β-2-Microglobulin (B2M) gene that suppresses or eliminates expression of a functional B2M protein.

59. The modified cell of claim 51, further comprising a genomic disruption in an endogenous Class II transactivator (CIITA) gene that suppresses or eliminates expression of a functional CIITA protein.

60. The modified cell of claim 51, further comprising a genomic disruption in an endogenous T-cell receptor alpha constant region (TRAC) gene that suppresses or eliminates expression of a functional TRAC protein and/or a genomic disruption in an endogenous TRBC1/2 gene that suppresses or eliminates expression of a functional TRBC1/2 protein.

61. The modified cell of claim 51, further comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB).

62. The modified cell of claim 61, wherein (A) a first exogenous polynucleotide encoding the first fusion protein subunit of the signal converter is inserted into a SOCS1 locus, and a second exogenous polynucleotide encoding the second fusion protein subunit of the signal converter is inserted into a ROSA26 locus; or (B) a first exogenous polynucleotide encoding the first fusion protein subunit of the signal converter is inserted into a ROSA26 locus, and a second exogenous polynucleotide encoding the second fusion protein subunit of the signal converter is inserted into a SOCS1 locus.

63. The modified cell of claim 61, the first fusion protein subunit of the signal converter comprises an amino acid sequence of SEQ ID NO: 71, and the second fusion protein subunit of the signal converter comprises an amino acid sequence of SEQ ID NO: 72; or
the first exogenous polynucleotide encodes an amino acid sequence comprising SEQ ID NO: 75, and the second exogenous polynucleotide encodes an amino acid sequence comprising SEQ ID NO: 76.

64. The modified cell of claim 51, further comprising an exogenous polynucleotide encoding a B2M-HLA-E fusion protein (B2M-HLA-E) comprising at least a portion of B2M fused with at least a portion of HLA-E; and wherein the exogenous polynucleotide encoding the B2M-HLA-E is inserted into a B2M locus; and wherein the B2M-HLA-E comprises an amino acid sequence of SEQ ID NO: 81.

65. The modified cell of claim 51, further comprising one or more exogenous polynucleotides encoding a TCRαβ which specifically binds KRAS G12V; wherein the one or more exogenous polynucleotides encoding the TCRαβ are inserted into a TRAC locus; and wherein the TCRαβ comprises an α chain having the amino acid sequence of SEQ ID NO: 79 and a β chain having the amino acid sequence of SEQ ID NO: 80, or the TCRαβ comprises an α chain having the amino acid sequence of SEQ ID NO: 82 and a β chain having the amino acid sequence of SEQ ID NO: 83.

66. The modified cell of claim 51, further comprising an exogenous polynucleotide encoding a suicide switch, which comprises a herpes simplex virus thymidine kinase (HSV-tk) polypeptide or a functional variant thereof capable of inducing cell death upon administration of Ganciclovir (GCV).

67. The modified cell of claim 66, wherein the suicide switch comprises an amino acid sequence of SEQ ID NO: 85; and wherein the suicide switch is inserted into a Rogi2 locus, or wherein the suicide switch is inserted into a FAS locus.

68. The modified cell of claim 51, wherein the αβT cell is an iPSC-derived αβT (iαβT) cell; or wherein the αβT cell is a αβT cell derived from a T-iPSC (T-iαβT); or wherein the αβT cell is a αβT cell differentiated from a αβT-iPSC (αβT-iαβT).

69. A pharmaceutical composition comprising the modified αβT cell of claim 51 and a pharmaceutically acceptable carrier.

70. A method of killing B cells which are CD19 positive and/or CD20 positive and have abnormal B cell function, comprising administering to a subject in need thereof a therapeutically effective amount of the modified αβT cell of claim 51.

71. A modified iPSC, αβT cell, or intermediate cell differentiated from an iPSC, comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein.

72. A modified iPSC, αβT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell; optionally wherein the genomic disruption is an inactivating nucleic acid mutation in the exon 2C, optionally wherein the mutation is an insertion, deletion, or substitution; and
wherein the genomic disruption within BIM is made by administering a guide RNA to said cell, and optionally wherein an endonuclease or a nucleic acid encoding the endonuclease is also administered to said cell, and optionally wherein the endonuclease is a Cas9 endonuclease or a MAD7 endonuclease.

73. A modified iPSC, αβT cell, or intermediate cell differentiated from an iPSC, comprising a genomic disruption in a target sequence of an endogenous SOCS1 gene that suppresses or eliminates expression of a functional SOCS1 protein, wherein the target sequence comprises SEQ ID NO: 17 or SEQ ID NO: 23; and optionally wherein the genomic disruption is an inactivating nucleic acid mutation, optionally wherein the mutation is an insertion, deletion, or substitution; and
wherein the genomic disruption within SOCS1 is made by administering a guide RNA to said cell, and optionally wherein an endonuclease or a nucleic acid encoding the endonuclease is also administered to said cell; and optionally wherein the endonuclease is a Cas9 endonuclease or a MAD7 endonuclease; and optionally wherein the guide RNA comprises the sgRNA or crRNA sequence of any one of SEQ ID NOs: 29 and 37, or comprises the guide sequence differing by no more than 3 nucleotides from SEQ ID NO: 5 or 11.

74. A modified iPSC, αβT cell, or intermediate cell differentiated from an iPSC, comprising a genomic disruption in a target sequence of an endogenous CISH gene that suppresses or eliminates expression of CISH, wherein the target sequence comprises SEQ ID NO: 24; and optionally wherein the genomic disruption is an inactivating nucleic acid mutation, optionally wherein the mutation is an insertion, deletion, or substitution; and
wherein the genomic disruption within CISH is made by administering a guide RNA to said cell, and optionally wherein an endonuclease or a nucleic acid encoding the endonuclease is also administered to said cell; and optionally wherein the endonuclease is a Cas9 endonuclease or a MAD7 endonuclease; and optionally wherein the guide RNA comprises the crRNA sequence of SEQ ID NO: 38 or comprises the guide sequence differing by no more than 3 nucleotides from SEQ ID NO: 12, and the endonuclease is a MAD7 endonuclease.

75. A modified iPSC, αβT cell, or intermediate cell differentiated from an iPSC, comprising one or more exogenous polynucleotides encoding a signal converter, wherein the signal converter comprises a first fusion protein subunit and a second fusion protein subunit, wherein the first fusion protein subunit comprises an extracellular domain (ECD) of interferon gamma receptor 1 (IFNGR1) and an intracellular domain of interleukin-2 receptor subunit gamma (IL2RG), and wherein the second fusion protein subunit comprises an ECD of interferon gamma receptor 2 (IFNGR2) and an intracellular domain of interleukin-2 receptor subunit beta (IL2RB); and optionally further comprising an exogenous polynucleotide encoding a chimeric antigen receptor (CAR) which specifically binds human CD19 and/or an exogenous polynucleotide encoding a chimeric antigen receptor (CAR) which specifically binds human CD20.

76. A gene editing system comprising one or more guide RNAs and an endonuclease or a nucleic acid encoding the endonuclease, wherein
(i) the one or more guide RNAs comprise one or more guide sequences that binds to a target sequence in an exon 2C of an endogenous BIM gene, wherein upon administering the gene editing system to a cell, the expression and/or function of BIM_{EL} and BIM_{L} splice variants are reduced in the cell relative to that in an unmodified control, and the expression and/or function of BIM_{S} splice variant is retained, and optionally the targe sequence comprises any one of SEQ ID NOs: 14, 20, 21, and 22;
(ii) the one or more guide RNAs comprise one or more guide sequences which binds to a target sequence in CISH gene that comprises SEQ ID NO: 24; wherein the endonuclease is a MAD7 endonuclease, and the one or more guide RNAs comprise the crRNA sequence of SEQ ID NO: 38 or the guide sequence differing by no more than 3 nucleotides from SEQ ID NO: 12; or
(iii) (1) the one or more guide RNAs comprise one or more guide sequences which binds to a target sequence in SOCS1 gene that comprises SEQ ID NO: 23; wherein the endonuclease is a MAD7 endonuclease, and the one or more guide RNAs comprise the crRNA sequence of SEQ ID NO: 37 or the guide sequence differing by no more than 3 nucleotides from SEQ ID NO: 11; or (2) the one or more guide RNAs comprise one or more guide sequences which binds to a target sequence in SOCS1 gene that comprises SEQ ID NO: 17, the endonuclease is a Cas9 endonuclease, and the one or more guide RNAs comprise the sgRNA sequence of SEQ ID NO: 29, or the guide sequence differing by no more than 3 nucleotides from any one of SEQ ID NO: 5.

77. A guide RNA comprising a CRISPR RNA (crRNA) sequence, wherein the crRNA sequence comprises a 2′-O-Methyl modification at each of the first three positions on the 5′ end and at each of the fourth to last, third to last, and second to last positions of the crRNA sequence on the 3′ end, and a phosphorothioate linkage between each base in the first three positions on the 5′ end and a phosphorothioate linkage between each base of the last three positions of the crRNA sequence at the 3′ end; and optionally wherein the guide RNA functions with a MAD7 endonuclease; and optionally wherein the crRNA is about 56 bases long.


SEQUENCE LISTING (94 sequences)

SEQ ID NO: 1 - RNA, 20 nt, synthetic construct
  source 1..20: mol_type = other RNA
gcactcacatcgctacatca

SEQ ID NO: 2 - RNA, 20 nt, synthetic construct
  source 1..20: mol_type = other RNA
tacccattgcactgagatag

SEQ ID NO: 3 - RNA, 20 nt, synthetic construct
  source 1..20: mol_type = other RNA
aggccacatagtgctgcaca

SEQ ID NO: 4 - RNA, 20 nt, synthetic construct
  source 1..20: mol_type = other RNA
tgtacagcagtggctggtgg

SEQ ID NO: 5 - RNA, 20 nt, synthetic construct
  source 1..20: mol_type = other RNA
agtagaatccgcaggcgtcc

SEQ ID NO: 6 - RNA, 20 nt, synthetic construct
  source 1..20: mol_type = other RNA
agttctgagtgtgaccgaga

SEQ ID NO: 7 - RNA, 20 nt, synthetic construct
  source 1..20: mol_type = other RNA
gcctccccagctcagacctg

SEQ ID NO: 8 - RNA, 20 nt, synthetic construct
  source 1..20: mol_type = other RNA
ggcctggcaaggaggacttg

SEQ ID NO: 9 - RNA, 20 nt, synthetic construct
  source 1..20: mol_type = other RNA
tggttgaaggcctggcaagg

SEQ ID NO: 10 - RNA, 21 nt, synthetic construct
  source 1..21: mol_type = other RNA
acacagacaggagcccagcac

SEQ ID NO: 11 - RNA, 21 nt, synthetic construct
  source 1..21: mol_type = other RNA
acctggatggcagccgcgaga

SEQ ID NO: 12 - RNA, 21 nt, synthetic construct
  source 1..21: mol_type = other RNA
ggtgtacagcagtggctggtg

SEQ ID NO: 13 - RNA, 20 nt, synthetic construct
  source 1..20: mol_type = other RNA
tgatgtagcgatgtgagtgc

SEQ ID NO: 14 - DNA, 20 bp, Homo sapiens
  source 1..20: mol_type = genomic DNA
ctatctcagtgcaatgggta

SEQ ID NO: 15 - DNA, 20 bp, Homo sapiens
  source 1..20: mol_type = genomic DNA
tgtgcagcactatgtggcct

SEQ ID NO: 16 - DNA, 20 bp, Homo sapiens
  source 1..20: mol_type = genomic DNA
ccaccagccactgctgtaca

SEQ ID NO: 17 - DNA, 20 bp, Homo sapiens
  source 1..20: mol_type = genomic DNA
ggacgcctgcggattctact

SEQ ID NO: 18 - DNA, 20 bp, Homo sapiens
  source 1..20: mol_type = genomic DNA
tctcggtcacactcagaact

SEQ ID NO: 19 - DNA, 20 bp, Homo sapiens
  source 1..20: mol_type = genomic DNA
caggtctgagctggggaggc

SEQ ID NO: 20 - DNA, 20 bp, Homo sapiens
  source 1..20: mol_type = genomic DNA
caagtcctccttgccaggcc

SEQ ID NO: 21 - DNA, 20 bp, Homo sapiens
  source 1..20: mol_type = genomic DNA
ccttgccaggccttcaacca

SEQ ID NO: 22 - DNA, 21 bp, Homo sapiens
  source 1..21: mol_type = genomic DNA
gtgctgggctcctgtctgtgt

SEQ ID NO: 23 - DNA, 21 bp, Homo sapiens
  source 1..21: mol_type = genomic DNA
tctcgcggctgccatccaggt

SEQ ID NO: 24 - DNA, 21 bp, Homo sapiens
  source 1..21: mol_type = genomic DNA
caccagccactgctgtacacc

SEQ ID NO: 25 - RNA, 100 nt, synthetic construct
  source 1..100: mol_type = other RNA
gcactcacatcgctacatcagttttagagctagaaatagcaagttaaaataaggctagtccgttatcaacttgaaaaagtggcaccgagtcggtgctttt

SEQ ID NO: 26 - RNA, 100 nt, synthetic construct
  source 1..100: mol_type = other RNA
tacccattgcactgagataggttttagagctagaaatagcaagttaaaataaggctagtccgttatcaacttgaaaaagtggcaccgagtcggtgctttt

SEQ ID NO: 27 - RNA, 100 nt, synthetic construct
  source 1..100: mol_type = other RNA
aggccacatagtgctgcacagttttagagctagaaatagcaagttaaaataaggctagtccgttatcaacttgaaaaagtggcaccgagtcggtgctttt

SEQ ID NO: 28 - RNA, 100 nt, synthetic construct
  source 1..100: mol_type = other RNA
tgtacagcagtggctggtgggttttagagctagaaatagcaagttaaaataaggctagtccgttatcaacttgaaaaagtggcaccgagtcggtgctttt

SEQ ID NO: 29 - RNA, 100 nt, synthetic construct
  source 1..100: mol_type = other RNA
agtagaatccgcaggcgtccgttttagagctagaaatagcaagttaaaataaggctagtccgttatcaacttgaaaaagtggcaccgagtcggtgctttt

SEQ ID NO: 30 - RNA, 100 nt, synthetic construct; identical to SEQ ID NO: 25
  source 1..100: mol_type = other RNA

SEQ ID NO: 31 - skipped

SEQ ID NO: 32 - RNA, 100 nt, synthetic construct
  source 1..100: mol_type = other RNA
agttctgagtgtgaccgagagttttagagctagaaatagcaagttaaaataaggctagtccgttatcaacttgaaaaagtggcaccgagtcggtgctttt

SEQ ID NO: 33 - RNA, 100 nt, synthetic construct
  source 1..100: mol_type = other RNA
gcctccccagctcagacctggttttagagctagaaatagcaagttaaaataaggctagtccgttatcaacttgaaaaagtggcaccgagtcggtgctttt

SEQ ID NO: 34 - RNA, 100 nt, synthetic construct
  source 1..100: mol_type = other RNA
ggcctggcaaggaggacttggttttagagctagaaatagcaagttaaaataaggctagtccgttatcaacttgaaaaagtggcaccgagtcggtgctttt

SEQ ID NO: 35 - RNA, 100 nt, synthetic construct
  source 1..100: mol_type = other RNA
tggttgaaggcctggcaagggttttagagctagaaatagcaagttaaaataaggctagtccgttatcaacttgaaaaagtggcaccgagtcggtgctttt

SEQ ID NO: 36 - RNA, 56 nt, synthetic construct
  source 1..56: mol_type = other RNA
gtcaaaagacctttggaatttctactcttgtagatacacagacaggagcccagcac

SEQ ID NO: 37 - RNA, 56 nt, synthetic construct
  source 1..56: mol_type = other RNA
gtcaaaagacctttggaatttctactcttgtagatacctggatggcagccgcgaga

SEQ ID NO: 38 - RNA, 56 nt, synthetic construct
  source 1..56: mol_type = other RNA
gtcaaaagacctttggaatttctactcttgtagatggtgtacagcagtggctggtg

SEQ ID NO: 39 - RNA, 20 nt, synthetic construct
  source 1..20: mol_type = other RNA
tttgctaccagatccccgct

SEQ ID NO: 40 - RNA, 23 nt, synthetic construct
  source 1..23: mol_type = other RNA
ccaaaccctctgagcaataagca

SEQ ID NO: 41 - RNA, 20 nt, synthetic construct
  source 1..20: mol_type = other RNA
gcactgccttgacttgcact

SEQ ID NO: 42 - RNA, 20 nt, synthetic construct
  source 1..20: mol_type = other RNA
gggggcatttacctccaagt

SEQ ID NO: 43 - RNA, 20 nt, synthetic construct
  source 1..20: mol_type = other RNA
tccactgaggctgaacggat

SEQ ID NO: 44 - RNA, 19 nt, synthetic construct
  source 1..19: mol_type = other RNA
ctgaaagtgcacgcggatg

SEQ ID NO: 45 - RNA, 24 nt, synthetic construct
  source 1..24: mol_type = other RNA
accgagaaggtagacaattgcagc

SEQ ID NO: 46 - RNA, 24 nt, synthetic construct
  source 1..24: mol_type = other RNA
agtttgacacatcctccattccca

SEQ ID NO: 47 - RNA, 20 nt, synthetic construct
  source 1..20: mol_type = other RNA
aatcacggaggtgaagggga

SEQ ID NO: 48 - RNA, 22 nt, synthetic construct
  source 1..22: mol_type = other RNA
gccccttctgtaggatggtagc

SEQ ID NO: 49 - RNA, 22 nt, synthetic construct
  source 1..22: mol_type = other RNA
ggggaaggagctcaggtagtcg

SEQ ID NO: 50 - RNA, 20 nt, synthetic construct
  source 1..20: mol_type = other RNA
agcacacaaccaggtggcag

SEQ ID NO: 51 - RNA, 20 nt, synthetic construct
  source 1..20: mol_type = other RNA
ggttccattacggccagcga

SEQ ID NO: 52 - RNA, 20 nt, synthetic construct
  source 1..20: mol_type = other RNA
ggccagctgccagaggtatg

SEQ ID NO: 53 - RNA, 20 nt, synthetic construct
  source 1..20: mol_type = other RNA
cgcaccccagctacctgttc

SEQ ID NO: 54 - RNA, 100 nt, synthetic construct
  source 1..100: mol_type = other RNA
cgtgagtaaacctgaatcttgttttagagctagaaatagcaagttaaaataaggctagtccgttatcaacttgaaaaagtggcaccgagtcggtgctttt

SEQ ID NO: 55 - RNA, 20 nt, synthetic construct
  source 1..20: mol_type = other RNA
cgtgagtaaacctgaatctt

SEQ ID NO: 56 - DNA, 20 bp, Homo sapiens
  source 1..20: mol_type = genomic DNA
aagattcaggtttactcacg

SEQ ID NO: 57 - RNA, 100 nt, synthetic construct
  source 1..100: mol_type = other RNA
gatattggcataagcctcccgttttagagctagaaatagcaagttaaaataaggctagtccgttatcaacttgaaaaagtggcaccgagtcggtgctttt

SEQ ID NO: 58 - RNA, 20 nt, synthetic construct
  source 1..20: mol_type = other RNA
gatattggcataagcctccc

SEQ ID NO: 59 - DNA, 20 bp, Homo sapiens
  source 1..20: mol_type = genomic DNA
gggaggcttatgccaatatc

SEQ ID NO: 60 - RNA, 100 nt, synthetic construct
  source 1..100: mol_type = other RNA
gattgctcaacaaccatgctgttttagagctagaaatagcaagttaaaataaggctagtccgttatcaacttgaaaaagtggcaccgagtcggtgctttt

SEQ ID NO: 61 - RNA, 20 nt, synthetic construct
  source 1..20: mol_type = other RNA
gattgctcaacaaccatgct

SEQ ID NO: 62 - DNA, 20 bp, Homo sapiens
  source 1..20: mol_type = genomic DNA
agcatggttgttgagcaatc

SEQ ID NO: 63 - protein, 228 aa, synthetic construct [sequence omitted]

SEQ ID NO: 64 - protein, 226 aa, synthetic construct
APPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 65 - protein, 5 aa, synthetic construct
FALEA

SEQ ID NO: 66 - protein, 5 aa, synthetic construct
LGKDT

SEQ ID NO: 67 - protein, 21 aa, synthetic construct
VVISVGSMGLIISLLCVYFWL

SEQ ID NO: 68 - protein, 25 aa, synthetic construct
IPWLGHLLVGLSGAFGFIILVYLLI

SEQ ID NO: 69 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 70 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 71 - protein, 340 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 72 - protein, 542 aa, synthetic construct
APPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQLGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 73 - protein, 17 aa, synthetic construct
MALLFLLPLVMQGVSRA

SEQ ID NO: 74 - protein, 21 aa, synthetic construct
MRPTLLWSLLLLLGVFAAAAA

SEQ ID NO: 75 - protein, 357 aa, synthetic construct
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 76 - protein, 563 aa, synthetic construct
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQLGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 77 - RNA, 20 nt, synthetic construct
  source 1..20: mol_type = other RNA
gaaacgtctgtgagcctctc

SEQ ID NO: 78 - RNA, 20 nt, synthetic construct
  source 1..20: mol_type = other RNA
cctttggcttggcttgtcag

SEQ ID NO: 79 - protein, 274 aa, synthetic construct
MHLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRGASQGGSEKLVFGKGTKLTVNPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 80 - protein, 306 aa, synthetic construct
MASRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLTSGGFDEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 81 - protein, 500 aa, synthetic construct
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 82 - protein, 260 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSDPRYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 83 - protein, 295 aa, synthetic construct
DSGVTQTPKHLITATGQRVTLRCSPRSGDLSVYWYQQSLDQGLQFLIQYYNGEERAKGNILERFSAQQFPDLHSELNLSSLELGDSALYFCASSPRGGDSILDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - protein, 22 aa, synthetic construct
GSGATNFSLLKQAGDVEENPGP

SEQ ID NO: 85 - protein, 331 aa, synthetic construct
MPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 86 - protein, 853 aa, synthetic construct
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLGSGATNFSLLKQAGDVEENPGPMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 87 - protein, 21 aa, synthetic construct
MALPVTALLLPLALLLHAARP

SEQ ID NO: 88 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 89 - protein, 45 aa, synthetic construct
TTTPAPRPPTPAPTIASQPLSLRPEACRPAAGGAVHTRGLDFACD

SEQ ID NO: 90 - protein, 24 aa, synthetic construct
IYIWAPLAGTCGVLLLSLVITLYC

SEQ ID NO: 91 - protein, 42 aa, synthetic construct
KRGRKKLLYIFKQPFMRPVQTTQEEDGCSCRFPEEEEGGCEL

SEQ ID NO: 92 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 465 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR